(19) United States
(12) Patent Application Publication
(10) Pub. No.: US 2022/0226397 A1
(43) Pub. Date: Jul. 21, 2022

(54) SYNERGISTIC COMBINATIONS OF ARGININE-PRODUCING BACTERIA AND/OR AMMONIA-CONSUMING BACTERIA AND CHECKPOINT INHIBITORS AND METHODS OF USE THEREOF

(71) Applicant: Synlogic Operating Company, Inc., Cambridge, MA (US)

(72) Inventors: Jose M. Lora, Boston, MA (US); Roger Geiger, Cugnasco (CH)

(21) Appl. No.: 17/610,741

(22) PCT Filed: May 13, 2020
(86) PCT No.: PCT/US2020/032588

§ 371 Date: Nov. 12, 2021

Related U.S. Application Data
(60) Provisional application No. 62/846,938, filed on May 13, 2019.

(51) Int. Cl. A61K 35/74; A61P 35/00; A61K 39/395; A61K 31/198

(52) U.S. Cl. CPC A61K 35/74; A61K 31/198; A61K 39/3955; A61P 35/00

(57) ABSTRACT

Disclosed herein are synergistic compositions and methods using arginine-producing bacteria and/or ammonia-consuming bacteria in combination with a checkpoint inhibitor for use in treating cancer.

### RELATED APPLICATIONS

This application claims priority to U.S. Provisional Application No. 62/846,938, filed on May 13, 2019, the entire contents of which are expressly incorporated herein by reference.

### SEQUENCE LISTING

The instant application contains a Sequence Listing which has been submitted electronically in ASCII format and is hereby incorporated by reference in its entirety. Said ASCII copy, created on May 12, 2020 is named 126046-04120_SL.txt and is 54,799 bytes in size.

### BACKGROUND

Current cancer therapies typically employ the use of immunotherapy, surgery, chemotherapy, radiation therapy, or some combination thereof (American Cancer Society). While these drugs have shown great benefits to cancer patients, many cancers remain difficult to treat using conventional therapies. Currently, many conventional cancer therapies are administered systemically and adversely affect healthy tissues, resulting in significant side effects. For example, many cancer therapies focus on activating the immune system to boost the patient's anti-tumor response (Kong et al., 2014). However, despite such therapies, the microenvironment surrounding tumors remains highly immune suppressive. In addition, systemic altered immunoregulation provokes immune dysfunction, including the onset of opportunistic autoimmune disorders and immune-related adverse events.

Major efforts have been made over the past few decades to develop cytotoxic drugs that specifically target cancer cells. In recent years, there has been a paradigm shift in oncology in which the clinical problem of cancer is considered not only to be the accumulation of genetic abnormalities in cancer cells but also the tolerance of these abnormal cells by the immune system. Consequently, recent anti-cancer therapies have been designed specifically to target the immune system rather than cancer cells. Such therapies aim to reverse the cancer immunotolerance and stimulate an effective antitumor immune response. For example, current immunotherapies include immunostimulatory molecules that are pattern recognition receptor (PRR) agonists or immunostimulatory monoclonal antibodies that target various immune cell populations that infiltrate the tumor microenvironment. However, despite their immune-targeted design, these therapies have been developed clinically as if they were conventional anticancer drugs, relying on systemic administration of the immunotherapeutic (e.g., intravenous infusions every 2-3 weeks). As a result, many current immunotherapies suffer from toxicity due to a high dosage requirement, and also often result in an undesired autoimmune response or other immune-related adverse events.

Overall, there is an unmet need for effective cancer therapies that are able to locally target poorly vascularized, hypoxic tumor regions specifically target cancerous cells, while minimally affecting normal tissues and boost the immune systems to fight the tumors, including avoiding or reversing the cancer immunotolerance.

### SUMMARY

The instant disclosure provides compositions and methods for treating and/or preventing cancer by locally: i) increasing arginine biosynthesis and/or ii) increasing ammonia consumption. Ammonia is highly toxic and generated during metabolism in all organs (Walker, 2012). In mammals, the healthy liver protects the body from ammonia by converting ammonia to non-toxic molecules, e.g., urea or glutamine, and preventing excess amounts of ammonia from entering the systemic circulation. Ammonia is also a source of nitrogen for amino acids, which are synthesized by various biosynthesis pathways. For example, arginine biosynthesis converts glutamate, which comprises one nitrogen atom, to arginine, which comprises four nitrogen atoms. Intermediate metabolites formed in the arginine biosynthesis pathway, such as citrulline, also incorporate nitrogen. Thus, enhancement of arginine biosynthesis and/or ammonia consumption may be used to incorporate excess nitrogen in the body into non-toxic molecules in order to increase anti-tumor response in cells.

The availability of L-arginine in tumors is also a key determinant of an efficient anti-tumor T cell response (Rodriguez, et al.; Bronte, et al.; Geiger, et al.). Consequently, elevation of typically low L-arginine levels within the tumor increases the anti-tumor responses of anti-tumor T cells. However, to achieve the desired effect, relatively high doses of L-arginine have to be administered to a subject. For example, a patient weighing 75 kg would need to ingest 150 g of L-arginine daily, which is nearly impracticable. Thus, there are currently no means available to locally increase intra-tumoral L-arginine levels.

In specific aspects, the disclosure relates to compositions comprising bacteria that overproduce arginine and/or consume ammonia, particularly in low-oxygen conditions, such as in a tumor micro-environment. It is surprisingly demonstrated herein that administering a bacteria which overproduces arginine and/or consumes ammonia, in combination with a checkpoint inhibitor, results in a striking synergistic effect against a tumor.

More specifically, the present disclosure provides a synthetic biology approach to developing an engineered Escherichia coli Nissle strain that colonizes tumors and continuously converts ammonia, a metabolic waste product that accumulates in tumors, into L-arginine. In certain embodiments, colonization of tumors with these L-arginine producing bacteria elevates intra-tumoral L-arginine concentrations, increases the amount of tumor-infiltrating T cells and demonstrates striking synergistic effects with PD-L1 blocking antibodies in the clearance of tumors. In certain embodiments, the anti-tumor effects of the living therapeutic are mediated by L-arginine and are dependent on T cells. In certain embodiments, the engineered microbial therapies enable metabolic modulation of the tumor microenvironment leading to enhanced efficacy of cancer immunotherapies.

In one embodiment, the disclosure provides a method of treating a tumor in a subject, the method comprising administering to the subject an arginine-producing bacterium in combination with a checkpoint inhibitor, wherein the combination of the bacterium and the checkpoint inhibitor causes a synergistic therapeutic effect on the tumor in the subject, thereby treating the tumor in the subject. In one preferred embodiment, the bacterium is also an ammonia-consuming bacterium.

In one preferred embodiment, the bacterium is capable of producing at least about 300 μM arginine in culture in vitro after about 3 hours. In one preferred embodiment, the bacterium is capable of producing at least about 100 μM arginine in culture in vitro after about 1.5 hours. In one preferred embodiment, the bacterium produces between about 10 μM to about 600 μM arginine in culture in vitro between about 0.5 hours to about 3 hours. In one preferred embodiment, the bacterium produces at least about 3 μg of arginine per gram of tumor. In one preferred embodiment, the bacterium between at least about 3 μg of arginine per gram of tumor to at least about 25 μg of arginine per gram of tumor.

In another embodiment, the bacterium comprises a deletion in an endogenous arginine repressor gene and expresses at least one exogenous arginine biosynthetic enzyme under the control of an inducible promoter. In one preferred embodiment, the bacterium comprises a deletion of an argR gene and insertion of an argA gene. In one preferred embodiment, the argA gene is argA^{fbr}. In one embodiment, the argA^{fbr} gene has at least 90% sequence identity to SEQ ID NO:30. In another embodiment, the argA^{fbr} gene has at least 95%, 96%, 97%, 98%, 99% or 100% sequence identity to SEQ ID NO:30. In one embodiment, the argR gene has at least 90% sequence identity to SEQ ID NO:41. In one embodiment, the argR gene has at least 95%, 96%, 97%, 98%, 99% or 100% sequence identity to SEQ ID NO:41.

In one embodiment, the disclosure provides a method of treating a tumor in a subject, the method comprising administering to the subject an ammonia-consuming bacterium in combination with a checkpoint inhibitor, wherein the combination of the bacterium and the checkpoint inhibitor causes a synergistic therapeutic effect on the tumor in the subject, thereby treating the tumor in the subject. In one preferred embodiment, the bacterium is also an arginine-producing bacterium.

In another embodiment, the synergistic therapeutic effect is a decrease in tumor volume of at least about 100 mm³ after about 30 days of administration. In another embodiment, the synergistic therapeutic effect is a decrease in tumor weight of at least two-fold after about 30 days of administration. In another embodiment, the synergistic therapeutic effect is inhibition of tumor growth for at least 30 days after administration. In another embodiment, the synergistic therapeutic effect is partial eradication of the tumor. In another embodiment, the synergistic therapeutic effect is complete eradication of the tumor.

In another embodiment, the disclosure provides a method wherein the synergistic therapeutic effect is: i) a decrease in tumor volume, ii) a decrease in tumor weight, iii) inhibition of tumor growth, iv) partial eradication of the tumor, and/or v) complete eradication of the tumor.

In another embodiment, the subject is a population of subjects, and wherein at least 35% of the subjects in the population of subjects exhibit partial eradication of the tumor. In another embodiment, the subject is a population of subjects, and wherein at least 35% of the subjects in the population of subjects exhibit complete eradication of the tumor.

In another embodiment, the method further comprises wherein administering the bacterium and the checkpoint inhibitor is concurrent or sequential.

In one embodiment, the combination of the bacterium and the checkpoint inhibitor causes the synergistic therapeutic effect on the tumor in the subject as compared to a therapeutic effect caused by administering the bacterium alone or the checkpoint inhibitor alone to a subject. In one embodiment, the administration is concurrent or sequential.

In another embodiment, the method further comprises selecting a subject who would benefit from an increase in therapeutic efficacy of the checkpoint inhibitor.

In another embodiment, the disclosure provides a method of increasing T-cell infiltration into a tumor in a subject, the method comprising administering to the subject an ammonia-consuming and/or arginine-producing bacterium in combination with a checkpoint inhibitor. In one embodiment, the bacterium comprises a deletion in an endogenous arginine repressor gene and expresses at least one exogenous arginine biosynthetic enzyme under the control of an inducible promoter. In one embodiment, the combination of the modified bacterium and the checkpoint inhibitor increases T cell infiltration into the tumor in the subject at least two-fold as compared to the T cell infiltration exhibited by administering the modified bacterium alone or the checkpoint inhibitor alone to the subject. In one embodiment, the combination of the modified bacterium and the checkpoint inhibitor causes a synergistic therapeutic effect on the tumor in the subject as compared to a therapeutic effect caused by administering the modified bacterium alone or the checkpoint inhibitor alone to a subject.

In another embodiment, combination of the bacterium and the checkpoint inhibitor causes a synergistic therapeutic effect on the tumor in the subject as compared to a therapeutic effect caused by administering the bacterium alone, or the checkpoint inhibitor alone, to a subject.

In another embodiment, wherein arginine levels in the tumor microenvironment (TME) are increased and ammonia levels in the TME are decreased. In another embodiment, wherein arginine levels in the tumor microenvironment (TME) are increased to greater than 30 μg of arginine per gram of tumor.

In another embodiment, the method further comprises wherein the T-cell response against the tumor is enhanced. In another embodiment, the method further comprises wherein the tumor is colonized by at least about 8,000 CD4⁺ T-cells per gram of tumor tissue. In another embodiment, the method further comprises wherein the tumor is colonized by at least about 11,000 CD8⁺ T-cells per gram of tumor tissue.

In one embodiment, the bacterium is an engineered bacterium.

### BRIEF DESCRIPTION OF THE FIGURES

FIGS. 1A, 1B, and 1C: 5×10⁵ MC38 colon adenocarcinoma cells were injected s.c. in C57BL/6 WT mice and tumors were allowed to grow for 11 days before treatments were initiated. Control mice (n=13) received daily an oral gavage of H₂O (100 μl) and every second day 200 μl of PBS i.p. The L-arginine group received daily an oral gavage of L-arginine (2 g/kg body weight in 100 μl) (n=19). The PD-L1 group received 200 μg of αPD-L1 mAb i.p. every second day (n=16). The Combo group received L-arginine (oral) and αPD-L1 (i.p.) (n=17). FIG. 1A depicts in vivo MC38 tumor growth (number of mice are indicated in the graph). Values represent mean tumor volume +/− SEM. FIG. 1C: Survival curves of different groups of mice. P values were determined by log-rank comparisons between the curves. FIG. 1B provide tumor growth curves of individual mice. P values were determined by two way ANOVA. *P<0.05, **P<0.01, ***P<0.001, ****P<0.0001, throughout.

FIGS. 2A, 2B, 2C, 2D, 2E, and 2F: A metabolically engineered strain of E coli Nissle was generated to produce high levels of L-arginine from within the tumor microenvironment. FIG. 2A: depicts a schematic of L-Arg-bacterial strain where the arginine biosynthesis negative regulator protein ArgR was deleted and the feedback resistant ArgA^{fbr} was introduced under the control of an anaerobic promoter. FIG. 2B depicts an in vitro assay performed with 1×10⁹ cells/mL of pre-induced L-Arg bacteria or non-engineered control bacteria (EcN) in M9 media+0.5% glucose+5 mM NH₄Cl. Reaction mixture was incubated at 37° C. with shaking for 3 h. Samples were taken at 0, 1.5 and 3 hours respectively and arginine production was measured by LC-MS/MS. Non-engineered EcN was used as a negative control. n=2 FIG. 2C provides a pre-induced EcN and L-Arg bacteria grown for 3 h in M9 media+0.5% glucose+5 mM NH₄Cl and then their total proteome was analyzed by LC-MS/MS. Volcano plot shows differences in protein levels. Red and blue dots represent proteins whose levels were significantly higher or lower in L-Arg bacteria as compared to EcN (P<0.01, Log 2|FC|>4). n=4. FIG. 2D provides an illustration of the L-arginine biosynthesis pathway. The color code displays the Log 2 foldchange as determined by LC-MS/MS shown in FIG. 2c. FIG. 2E depicts MC38 tumors established and treated via intratumoral injection with 5×10⁶ CFUs of either L-Arg bacteria or EcN. Tumors were harvested and homogenized after 24, 72 and 120 h and bacterial abundance was measured by colony forming unit (CFU) assay. FIG. 2F depicts MC38 tumors established and treated via intratumoral injection with 5×10⁶ CFUs of either L-Arg bacteria or EcN. Tumors were harvested and homogenized after 24 h and arginine levels were measured by LC-MS/MS. P values were determined using a two-tailed t test.

FIGS. 3A, 3B, 3C, and 3D: H&E staining and CD3 immunohistochemistry of control MC38 tumors and tumors colonized with L-Arg bacteria and EcN (72 h). MC38 tumors were established in C57BL/6 WT mice and treated via intratumoral injection with 5×10⁶ CFUs of either L-Arg bacteria or EcN. (FIG. 3A) After 72 h, tumors were harvested, fixed and stained. Control tumors have low numbers of CD3-positive T-cells. EcN colonized tumors show similar numbers of T-cells, while tumors colonized with L-Arg bacteria are rich in T cells (bar represents 20 micrometer, arrow=CD3-positive T-cells). MC38 tumors were established in C57BL/6 WT mice and treated via intratumoral injection with 5×10⁶ CFUs of either L-Arg bacteria or EcN. After 24 h, tumors were harvested, dissociated and immune infiltrates were purified and stained with monoclonal antibodies to CD4 and CD8. (FIG. 3B). The number of T cells per gram tumor tissue was quantified by flow cytometry. P values were determined using a two-tailed t test. n=8 for Ctrl bacteria, n=11 for L-arg bacteria. Data are from three independent experiments. MC38 tumors were established in C57BL/6 WT mice. Tumors of the control group (n=15) were treated with 5×10⁶ CFU Ctrl bacteria (EcN)(i.t.) twice a week. Tumors of the L-arginine group (n=11) were treated with 5×10⁶ CFU L-Arg bacteria (i.t.) twice a week. A third group (n=17) received αPD-L1 mAb i.p. and 5×10⁶ CFU Ctrl bacteria i.t. (Ctrl-bacteria+αPD-L1). The combo group (n=16) received αPD-L1 mAb i.p. and 5×10⁶ L-Arg bacteria i.t (L-arg-bacteria+αPD-L1). FIG. 3C1: In vivo MC38 tumor growth (number of mice are indicated in the graph). Values represent mean tumor volume +/− SEM. A t-test was applied. FIG. 3C2: Survival curves of mice. A log-rank test comparison between curves was applied. One representative experiment out of two is shown. FIG. 3D depicts tumor growth curves in individual mice. *p<0.05 as determined by non-parametric t test statistical analysis in (left panel) and log-rank test comparison between curves in the right panel.

FIGS. 4A, 4B, 4C, 4D, and 4E: Mice in which tumors completely regressed (FIG. 3B) were re-challenged with 5×10⁵ MC38 colon adenocarcinoma cells 90 days after the first injection of MC38 cells. As a control 5×10⁵ MC38 cells were injected into naïve C57BL/6 WT mice. As a control 5×10⁵ MC38 cells were injected into naïve C57BL/6 WT mice. Mice that were re-challenged with MC38 (FIG. 4A) were challenged with 5×10⁵ B16 melanoma cells (FIG. 4B). MC38 tumors were established in C57BL/6 WT mice and in CD3e^{-/-} mice and treated via intratumoral injection with 5×10⁶ CFUs of either L-Arg bacteria or EcN (FIG. 4C). Tumors were harvested and homogenized after 24 hours and bacterial abundance was measured by CFU assay. FIG. 4D provides MC38 tumors that were established in CD3e^{-/-} mice. When tumors started to be visible and palpable, a homogenous group of tumor bearing mice was chosen for the different treatments. Tumors of the control group were treated with 5×10⁶ CFU Ctrl bacteria (it) twice a week. Tumors of the L-arginine group were treated with 5×10⁶ CFU L-Arg bacteria (i.t.) twice a week. A third group received αPD-L1 mAb i.p. and 5×10⁶ CFU Ctrl bacteria i.t. (Ctrl bacteria+αPD-L1). The Combo group (n=5) received αPD-L1 i.p. and 5×10⁶ L-Arg bacteria i.t (L-arg-bacteria+αPD-L1). Tumor growth over time. n=5 for each group. A t-test was applied. FIG. 4E shows survival curves of mice from FIG. 4D.

### DETAILED DESCRIPTION

The instant disclosure relates to synergistic compositions and methods comprising bacteria that overproduce arginine and/or consume ammonia, particularly in low-oxygen conditions, such as in a tumor micro-environment. It is surprisingly demonstrated herein that administering a bacteria which overproduces arginine and/or consumes ammonia, in combination with a checkpoint inhibitor, results in a striking synergistic effect against a tumor.

In particular, the instant disclosure provides a synthetic biology approach to develop an engineered probiotic Escherichia coli Nissle 1917 strain that colonizes tumors and continuously converts ammonia, a metabolic waste product that accumulates in tumors, into L-arginine. Colonization of tumors with these L-arginine producing bacteria elevates intra-tumoral L-arginine concentrations, increases the amount of tumor-infiltrating T cells and has striking synergistic effects with PD-L1 blocking antibodies in the clearance of tumors. The anti-tumor effect of the living therapeutic is mediated by L-arginine and is dependent on T cells. The engineered microbial therapies enable metabolic modulation of the tumor microenvironment leading to enhanced efficacy of immunotherapies, such as checkpoint inhibitors.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

“Ammonia” is used to refer to gaseous ammonia (NH₃), ionic ammonia (NH₄⁺), or a mixture thereof. In bodily fluids, gaseous ammonia and ionic ammonium exist in equilibrium: NH₃+H⁺↔NH₄⁺. Some clinical laboratory tests analyze total ammonia (NH₃+NH₄⁺) (Walker, 2012). In any embodiment of the disclosure, unless otherwise indicated, “ammonia” may refer to gaseous ammonia, ionic ammonia, and/or total ammonia.

“Detoxification” of ammonia is used to refer to the process or processes, natural or synthetic, by which toxic ammonia is removed and/or converted into one or more non-toxic molecules, including but not limited to: arginine, citrulline, methionine, histidine, lysine, asparagine, glutamine, tryptophan, or urea. The urea cycle, for example, enzymatically converts ammonia into urea for removal from the body in the urine. Because ammonia is a source of nitrogen for many amino acids, which are synthesized via numerous biochemical pathways, enhancement of one or more of those amino acid biosynthesis pathways may be used to incorporate excess nitrogen into non-toxic molecules. For example, arginine biosynthesis converts glutamate, which comprises one nitrogen atom, to arginine, which comprises four nitrogen atoms, thereby incorporating excess nitrogen into non-toxic molecules. In humans, arginine is not reabsorbed from the large intestine, and as a result, excess arginine in the large intestine is not considered to be harmful. Likewise, citrulline is not reabsorbed from the large intestine, and as a result, excess citrulline in the large intestine is not considered to be harmful. Arginine biosynthesis may also be modified to produce citrulline as an end product; citrulline comprises three nitrogen atoms and thus the modified pathway is also capable of incorporating excess nitrogen into non-toxic molecules.

As used herein, the term “ammonia-consuming bacterium” refers to a bacterium capable of reducing excess ammonia and/or converting ammonia and/or nitrogen into arginine byproducts. As used herein, the term “arginine-producing bacterium” refers to a bacterium capable of producing arginine and/or converting ammonia and/or nitrogen into arginine byproducts. In one embodiment, an ammonia-consuming bacterium is also an arginine-producing bacterium. In one embodiment, an arginine-producing bacterium is also an ammonia-consuming bacterium. In one embodiment, an ammonia-consuming bacterium is a modified bacterium. In one embodiment, an arginine-producing bacterium is a modified bacterium.

In one embodiment, a bacterium is a naturally non-pathogenic bacterium. In one embodiment, a bacterium is a commensal bacterium. In one embodiment, a bacterium is a probiotic bacterium. In one embodiment, a bacterium is a naturally pathogenic bacterium that is modified or mutated to reduce or eliminate pathogenicity.

As used herein, bacteria that “overproduce” arginine or an intermediate byproduct, e.g., citrulline, refer to bacteria that comprise a mutant arginine regulon. For example, the engineered bacteria may comprise a feedback resistant form of ArgA, and when the arginine feedback resistant ArgA is expressed, are capable of producing more arginine and/or intermediate byproduct than unmodified bacteria of the same subtype under the same conditions. The genetically engineered bacteria may alternatively or further comprise a mutant arginine regulon comprising one or more nucleic acid mutations in at least one ARG box for each of the operons that encode the arginine biosynthesis enzymes. The genetically engineered bacteria may alternatively or further comprise a mutant or deleted arginine repressor. In one embodiment, the genetically engineered bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more arginine than unmodified bacteria of the same subtype under the same conditions. In one embodiment, the genetically engineered bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more citrulline or other intermediate byproduct than unmodified bacteria of the same subtype under the same conditions. In one embodiment, the mRNA transcript levels of one or more of the arginine biosynthesis genes in the genetically engineered bacteria are at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold higher than the mRNA transcript levels in unmodified bacteria of the same subtype under the same conditions. In certain embodiments, the unmodified bacteria will not have detectable levels of arginine, intermediate byproduct, and/or transcription of the gene(s) in such operons. However, protein and/or transcription levels of arginine and/or intermediate byproduct will be detectable in the corresponding genetically engineered bacterium having the mutant arginine regulon. Transcription levels may be detected by directly measuring mRNA levels of the genes. Methods of measuring arginine and/or intermediate byproduct levels, as well as the levels of transcript expressed from the arginine biosynthesis genes, are known in the art. Arginine and citrulline, for example, may be measured by mass spectrometry.

“Arginine regulon,” “arginine biosynthesis regulon,” and “arg regulon” are used interchangeably to refer to the collection of operons in a given bacterial species that comprise the genes encoding the enzymes responsible for converting glutamate to arginine and/or intermediate metabolites, e.g., citrulline, in the arginine biosynthesis pathway. The arginine regulon also comprises operators, promoters, ARG boxes, and/or regulatory regions associated with those operons.

The arginine regulon includes, but is not limited to, the operons encoding the arginine biosynthesis enzymes N-acetylglutamate synthetase, N-acetylglutamate kinase, N-acetylglutamylphosphate reductase, acetylornithine aminotransferase, N-acetylornithinase, ornithine transcarbamylase, argininosuccinate synthase, argininosuccinate lyase, carbamoylphosphate synthase, operators thereof, promoters thereof, ARG boxes thereof, and/or regulatory regions thereof. In one embodiment, the arginine regulon comprises an operon encoding ornithine acetyltransferase and associated operators, promoters, ARG boxes, and/or regulatory regions, either in addition to or in lieu of N-acetylglutamate synthetase and/or N-acetylornithinase. In one embodiment, one or more operons or genes of the arginine regulon may be present on a plasmid in the bacterium. In one embodiment, a bacterium may comprise multiple copies of any gene or operon in the arginine regulon, wherein one or more copies may be mutated or otherwise altered as described herein.

One gene may encode one enzyme, e.g., N-acetylglutamate synthetase (argA). Two or more genes may encode distinct subunits of one enzyme, e.g., subunit A and subunit B of carbamoylphosphate synthase (carA and carB). In some bacteria, two or more genes may each independently encode the same enzyme, e.g., ornithine transcarbamylase (argF and argI). In some bacteria, the arginine regulon includes, but is not limited to, argA, encoding N-acetylglutamate synthetase; argB, encoding N-acetylglutamate kinase; argC, encoding N-acetylglutamylphosphate reductase; argD, encoding acetylornithine aminotransferase; argE, encoding N-acetylornithinase; argG, encoding argininosuccinate synthase; argH, encoding argininosuccinate lyase; one or both of argF and argI, each of which independently encodes ornithine transcarbamylase; carA, encoding the small subunit of carbamoylphosphate synthase; carB, encoding the large subunit of carbamoylphosphate synthase; operons thereof; operators thereof; promoters thereof; ARG boxes thereof; and/or regulatory regions thereof. In one embodiment, the arginine regulon comprises argJ, encoding ornithine acetyltransferase (either in addition to or in lieu of N-acetylglutamate synthetase and/or N-acetylornithinase), operons thereof, operators thereof, promoters thereof, ARG boxes thereof, and/or regulatory regions thereof.

“Arginine operon,” “arginine biosynthesis operon,” and “arg operon” are used interchangeably to refer to a cluster of one or more of the genes encoding arginine biosynthesis enzymes under the control of a shared regulatory region comprising at least one promoter and at least one ARG box. In one embodiment, the one or more genes are co-transcribed and/or co-translated. Any combination of the genes encoding the enzymes responsible for arginine biosynthesis may be organized, naturally or synthetically, into an operon. For example, in B. subtilis, the genes encoding N-acetylglutamylphosphate reductase, N-acetylglutamate kinase, N-acetylornithinase, N-acetylglutamate kinase, acetylornithine aminotransferase, carbamoylphosphate synthase, and ornithine transcarbamylase are organized in a single operon, argCAEBD-carAB-argF, under the control of a shared regulatory region comprising a promoter and ARG boxes. In E. coli K12 and Nissle, the genes encoding N-acetylornithinase, N-acetylglutamylphosphate reductase, N-acetylglutamate kinase, and argininosuccinate lyase are organized in two bipolar operons, argECBH. The operons encoding the enzymes responsible for arginine biosynthesis may be distributed at different loci across the chromosome. In unmodified bacteria, each operon may be repressed by arginine via ArgR. In one embodiment, arginine and/or intermediate byproduct production may be altered in the genetically engineered bacteria of the disclosure by modifying the expression of the enzymes encoded by the arginine biosynthesis operons as provided herein. Each arginine operon may be present on a plasmid or bacterial chromosome. In addition, multiple copies of any arginine operon, or a gene or regulatory region within an arginine operon, may be present in the bacterium, wherein one or more copies of the operon or gene or regulatory region may be mutated or otherwise altered as described herein. In one embodiment, the genetically engineered bacteria are engineered to comprise multiple copies of the same product (e.g., operon or gene or regulatory region) to enhance copy number or to comprise multiple different components of an operon performing multiple different functions.

“ARG box consensus sequence” refers to an ARG box nucleic acid sequence, the nucleic acids of which are known to occur with high frequency in one or more of the regulatory regions of argR, argA, argB, argC, argD, argE, argF, argG, argH, argI, argJ, carA, and/or carB. As described above, each arg operon comprises a regulatory region comprising at least one 18-nucleotide imperfect palindromic sequence, called an ARG box, that overlaps with the promoter and to which the repressor protein binds (Tian et al., 1992). The nucleotide sequences of the ARG boxes may vary for each operon, and the consensus ARG box sequence is A/T nTGAAT A/T A/T T/A T/A ATTCAn T/A (SEQ ID NO:39) (Maas, 1994). The arginine repressor binds to one or more ARG boxes to actively inhibit the transcription of the arginine biosynthesis enzyme(s) that are operably linked to that one or more ARG boxes.

“Mutant arginine regulon” or “mutated arginine regulon” is used to refer to an arginine regulon comprising one or more nucleic acid mutations that reduce or eliminate arginine-mediated repression of each of the operons that encode the enzymes responsible for converting glutamate to arginine and/or an intermediate byproduct, e.g., citrulline, in the arginine biosynthesis pathway, such that the mutant arginine regulon produces more arginine and/or intermediate byproduct than an unmodified regulon from the same bacterial subtype under the same conditions. In one embodiment, the genetically engineered bacteria comprise an arginine feedback resistant N-acetylglutamate synthase mutant, e.g., argA^{fbr}, and a mutant arginine regulon comprising one or more nucleic acid mutations in at least one ARG box for one or more of the operons that encode the arginine biosynthesis enzymes N-acetylglutamate kinase, N-acetylglutamylphosphate reductase, acetylornithine aminotransferase, N-acetylornithinase, ornithine transcarbamylase, argininosuccinate synthase, argininosuccinate lyase, and carbamoylphosphate synthase, thereby derepressing the regulon and enhancing arginine and/or intermediate byproduct biosynthesis. In one embodiment, the genetically engineered bacteria comprise a mutant arginine repressor comprising one or more nucleic acid mutations such that arginine repressor function is decreased or inactive, or the genetically engineered bacteria do not have an arginine repressor (e.g., the arginine repressor gene has been deleted), resulting in derepression of the regulon and enhancement of arginine and/or intermediate byproduct biosynthesis. In one embodiment, the genetically engineered bacteria comprise an arginine feedback resistant N-acetylglutamate synthase mutant, e.g., argA^{fbr}, a mutant arginine regulon comprising one or more nucleic acid mutations in at least one ARG box for each of the operons that encode the arginine biosynthesis enzymes, and/or a mutant or deleted arginine repressor. In one embodiment, the genetically engineered bacteria comprise an arginine feedback resistant N-acetylglutamate synthase mutant, e.g., argA^{fbr} and a mutant arginine regulon comprising one or more nucleic acid mutations in at least one ARG box for each of the operons that encode the arginine biosynthesis enzymes. In one embodiment, the genetically engineered bacteria comprise an arginine feedback resistant N-acetylglutamate synthase mutant, e.g., argA^{fbr} and a mutant or deleted arginine repressor. In one embodiment, the mutant arginine regulon comprises an operon encoding wild-type N-acetylglutamate synthetase and one or more nucleic acid mutations in at least one ARG box for said operon. In one embodiment, the mutant arginine regulon comprises an operon encoding wild-type N-acetylglutamate synthetase and mutant or deleted arginine repressor. In one embodiment, the mutant arginine regulon comprises an operon encoding ornithine acetyltransferase (either in addition to or in lieu of N-acetylglutamate synthetase and/or N-acetylornithinase) and one or more nucleic acid mutations in at least one ARG box for said operon.

The ARG boxes overlap with the promoter in the regulatory region of each arginine biosynthesis operon. In the mutant arginine regulon, the regulatory region of one or more arginine biosynthesis operons is sufficiently mutated to disrupt the palindromic ARG box sequence and reduce ArgR binding, but still comprises sufficiently high homology to the promoter of the non-mutant regulatory region to be recognized as the native operon-specific promoter. The operon comprises at least one nucleic acid mutation in at least one ARG box such that ArgR binding to the ARG box and to the regulatory region of the operon is reduced or eliminated. In one embodiment, bases that are protected from DNA methylation and bases that are protected from hydroxyl radical attack during ArgR binding are the primary targets for mutations to disrupt ArgR binding. The promoter of the mutated regulatory region retains sufficiently high homology to the promoter of the non-mutant regulatory region such that RNA polymerase binds to it with sufficient affinity to promote transcription of the operably linked arginine biosynthesis enzyme(s). In one embodiment, the G/C:A/T ratio of the promoter of the mutant differs by no more than 10% from the G/C:A/T ratio of the wild-type promoter. The promoter retains sufficiently high homology to the non-mutant promoter such that RNA polymerase binds with sufficient affinity to promote transcription.

The wild-type genomic sequences comprising ARG boxes and mutants thereof for each arginine biosynthesis operon in E. coli Nissle are shown in Table 1. For exemplary wild-type sequences, the ARG boxes are indicated in italics, and the start codon of each gene is [symbol] . The RNA polymerase binding sites are underlined (Cunin, 1983; Maas, 1994). In some embodiments, the underlined sequences are not altered. Bases that are protected from DNA methylation during ArgR binding are highlighted, and bases that are protected from hydroxyl radical attack during ArgR binding are bolded (Charlier et al., 1992). The highlighted and bolded bases are the primary targets for mutations to disrupt ArgR binding.

TABLE 1

| | Sequence |
|---|---|
| argA WT | 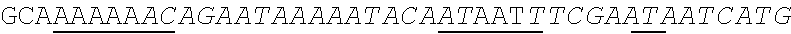 |
| (SEQ ID NO: 1) | 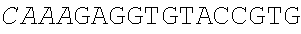 |
| argA mutant | gcaaaaaaacactttaaaaacttaataatttcctnaatcacttaaagaggtgtaccgtg |
| (SEQ ID NO: 2) | |
| argI WT | 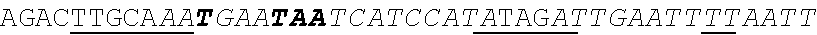 |
| (SEQ ID NO: 3) | 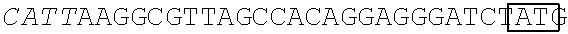 |
| argI mutant | agacttgcaaacttatacttatccatatagattttgttttaatttgttaaggcgttagccacaggaggga |
| (SEQ ID NO: 4) | tctatg |
| argCBH WT | 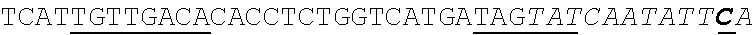 |
| (SEQ ID NO: 5) | 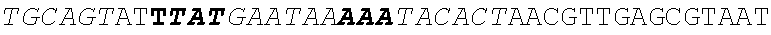 |
| | AAAACCCACCAGCCGTAAGGTGAATGTTTTACGTTTAACC |
| | 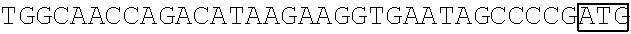 |
| argCBH mutant | tcattgttgacacacctctggtcatgatagtatcaaacttcatgggatatttatctttaaaaatacttga |
| (SEQ ID NO: 6) | acgttgagcgtaataaaacccaccagccgtaaggtgaatgttttacgtttaacctggcaaccagac |
| | ataagaaggtgaatagccccgatg |
| argE WT | CATCGGGGCTATTCACCTTCTTATGTCTGGTTGCCAGGTTA |
| (SEQ ID NO: 7) | AACGTAAAACATTCACCTTACGGCTGGTGGGTTTTATTACG |
| | 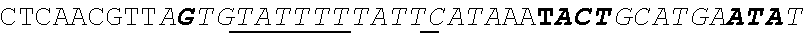 |
| | 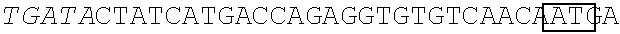 |
| argE mutant | catcggggctattcaccttcttatgtctggttgccaggttaaacgtaaaacattcaccttacggctgg |
| (SEQ ID NO: 8) | tgggttttattacgctcaacgttcaagtatttttaaagataaatatcccatgaagtttgatactatcatga |
| | ccagaggtgtgtcaacaatga |
| carAB WT | 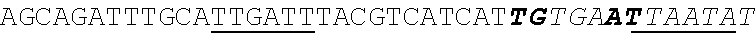 |
| (SEQ ID NO: 9) | 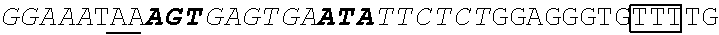 |
| carAB mutant | agcagatttgcattgatttacgtcatcattgtcttttaatatcttaataactggagtgacgtttctctgga |
| (SEQ ID NO: 10) | gggtgttttg |
| argD WT | 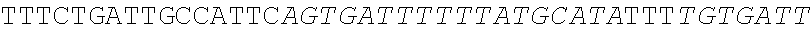 |
| (SEQ ID NO: 11) | 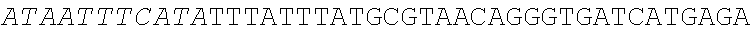 |
| | TG |
| argD mutant | tactgattgccattcagtctattttacttatagtctttataatcttatatttatttatgcgtaacagggt |
| (SEQ ID NO: 12) | gatcatgagatg |
| argG WT | 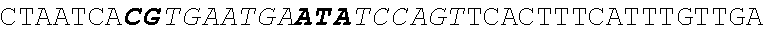 |
| (SEQ ID NO: 13) | ATACTTTTACCTTCTCCTGCTTTCCCTTAAGCGCATTATTTT |
| | ACAAAAAACACACTAAACTCTTCCTGTCTCCGATAAAAGA |
| | 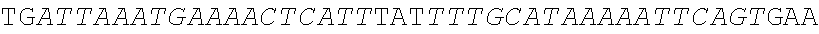 |
| | AGCAGAAATCCAGGCTCATCATCAGTTAATTAAGCAGGGT |
| | 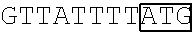 |
| argG mutant | ctaatcaccttaatgaatcttcagttcactttcatttgttgaatacaccttctcctgctttcccttaag |
| (SEQ ID NO: 14) | cgcattattttacaaaaaacacactaaactcttcctgtctccgataaaagatgatcttatgaaaaccttt |
| | ttatttcttataaaaatcttgtgaaagcagaaatccaggctcatcatcagttaattaagcagggtgtta |
| | ttttatg |
| argG mutant | cctgaaacgtggcaaattctactcgttttgggtaaaaaatgcaaatactgctgggatttggtgtacc |
| (SEQ ID NO: 15) | gagacgggacgtaaaatctgcaggcattatagtgatccacgccacattttgtcaacgtttattgcta |
| | atcattgacggctagctcagtcctaggtacagtgctagcACCCGTTTTTTTGGGCT |
| | AGAAATAATTTTGTTTAACTTTAAGAAGGAGATATACATA |
| | CCC |

In one embodiment, more than one ARG box may be present in a single operon. In one aspect of these embodiments, at least one of the ARG boxes in an operon is altered to produce the requisite reduced ArgR binding to the regulatory region of the operon. In an alternate aspect of these embodiments, each of the ARG boxes in an operon is altered to produce the requisite reduced ArgR binding to the regulatory region of the operon. One of skill in the art would appreciate that the number of ARG boxes per regulatory region may vary across bacteria, and the nucleotide sequences of the ARG boxes may vary for each operon. For example, the carAB operon in E. coli Nissle comprises two ARG boxes, and one or both ARG box sequences may be mutated. The argG operon in E. coli Nissle comprises three ARG boxes, and one, two, or three ARG box sequences may be mutated, disrupted, or deleted. In some embodiments, all three ARG box sequences are mutated, disrupted, or deleted, and a constitutive promoter, e.g., BBa_J23100, is inserted in the regulatory region of the argG operon. One of skill in the art would appreciate that the number of ARG boxes per regulatory region may vary across bacteria, and the nucleotide sequences of the ARG boxes may vary for each operon.

An exemplary embodiment of a constitutively expressed argG construct in E. coli Nissle is depicted in Table 2. Table 2 depicts the wild-type genomic sequence of the regulatory region and 5′ portion of the argG gene in E. coli Nissle, and a constitutive mutant thereof. The promoter region of each sequence is underlined, and a 5′ portion of the argG gene is [symbol] . In the wild-type sequence, ArgR binding sites are in uppercase and underlined. In the mutant sequence, the 5′ untranslated region is in uppercase and underlined. Bacteria expressing argG under the control of the constitutive promoter are capable of producing arginine. Bacteria expressing argG under the control of the wild-type, ArgR-repressible promoter are capable of producing citrulline.

TABLE 2

| | |
|---|---|
| Wild-type argG | gtgatccacgccacagtcaacgtttattgctaatcaCGTGAATGAATATCCAGTtc |
| (SEQ ID NO: 16) | actttcatttgttgaatacttttaccttctcctgctttcccttagcgcattattttacaaaaaacacactaaac |
| | tcttcctgtctccgataaaagatgATTAAATGAAAACTCATTtatTTTGCATAA |
| | 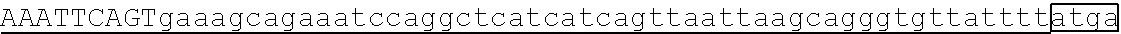 |
| | 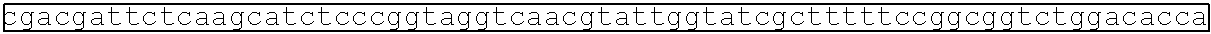 |
| | 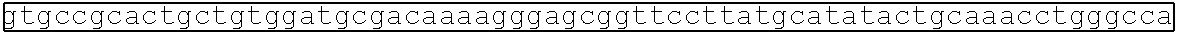 |
| | 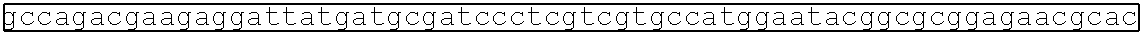 |
| | 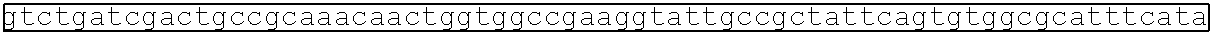 |
| | 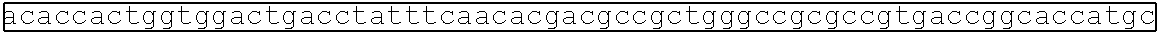 |
| | 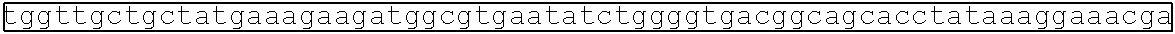 |
| | 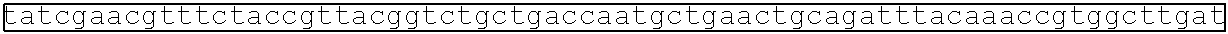 |
| | 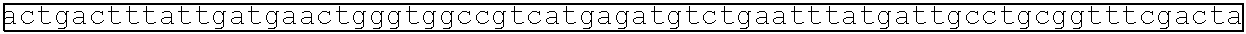 |
| | 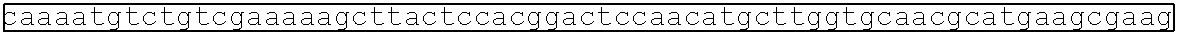 |
| | 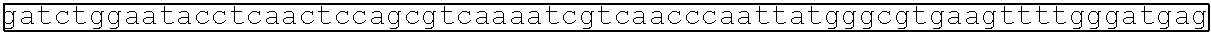 |
| | 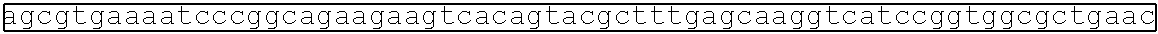 |
| | 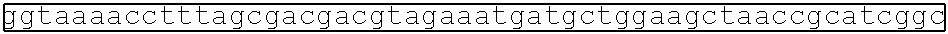 |
| Constitutive argG | ttgacggctagctcagtcctaggtacagtgctagcACCCGTTTTTTTGGGCTAGAA |
| (SEQ ID NO: 17) | 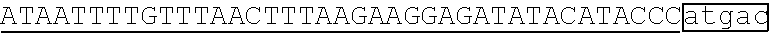 |
| | 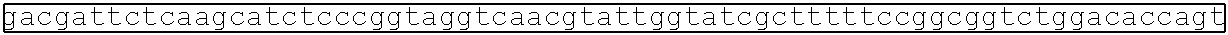 |
| | 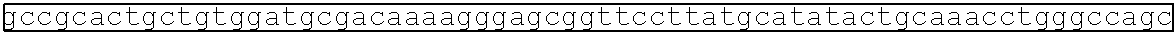 |
| | 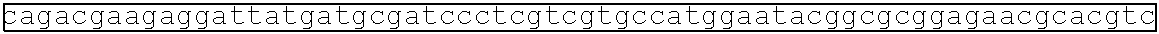 |
| | 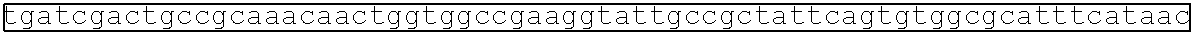 |
| | 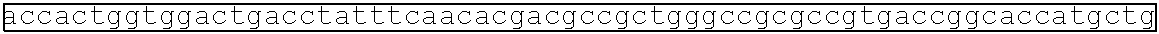 |
| | 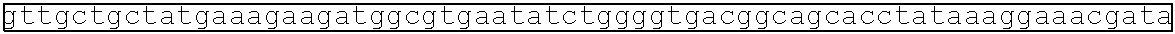 |
| | 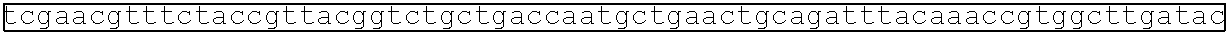 |
| | 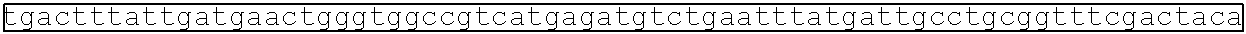 |
| | 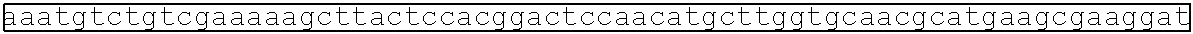 |
| | 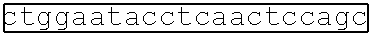 |

In some embodiments, the ArgR binding affinity to a mutant ARG box or regulatory region of an operon is at least about 50% lower, at least about 60% lower, at least about 70% lower, at least about 80% lower, at least about 90% lower, or at least about 95% lower than the ArgR binding affinity to an unmodified ARG box and regulatory region in bacteria of the same subtype under the same conditions. In some embodiments, the reduced ArgR binding to a mutant ARG box and regulatory region increases mRNA expression of the gene(s) in the associated operon by at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold.

“Reduced” ArgR binding is used to refer to a reduction in repressor binding to an ARG box in an operon or a reduction in the total repressor binding to the regulatory region of said operon, as compared to repressor binding to an unmodified ARG box and regulatory region in bacteria of the same subtype under the same conditions. In one embodiment, ArgR binding to a mutant ARG box and regulatory region of an operon is at least about 50% lower, at least about 60% lower, at least about 70% lower, at least about 80% lower, at least about 90% lower, or at least about 95% lower than ArgR binding to an unmodified ARG box and regulatory region in bacteria of the same subtype under the same conditions. In one embodiment, reduced ArgR binding to a mutant ARG box and regulatory region results in at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold increased mRNA expression of the one or more genes in the operon.

“ArgR” or “arginine repressor” is used to refer to a protein that is capable of suppressing arginine biosynthesis by regulating the transcription of arginine biosynthesis genes in the arginine regulon. When expression of the gene that encodes for the arginine repressor protein (“argR”) is increased in a wild-type bacterium, arginine biosynthesis is decreased. When expression of argR is decreased in a wild-type bacterium, or if argR is deleted or mutated to inactivate arginine repressor function, arginine biosynthesis is increased.

Bacteria that “lack any functional ArgR” and “ArgR deletion bacteria” are used to refer to bacteria in which each arginine repressor has significantly reduced or eliminated activity as compared to unmodified arginine repressor from bacteria of the same subtype under the same conditions. Reduced or eliminated arginine repressor activity can result in, for example, increased transcription of the arginine biosynthesis genes and/or increased concentrations of arginine and/or intermediate byproducts, e.g., citrulline. Bacteria in which arginine repressor activity is reduced or eliminated can be generated by modifying the bacterial argR gene or by modifying the transcription of the argR gene. For example, the chromosomal argR gene can be deleted, can be mutated, or the argR gene can be replaced with an argR gene that does not exhibit wild-type repressor activity.

“Operably linked” refers a nucleic acid sequence, e.g., a gene encoding feedback resistant ArgA, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

An “inducible promoter” refers to a regulatory region that is operably linked to one or more genes, wherein expression of the gene(s) is increased in the presence of an inducer of said regulatory region. In one embodiment, the genetically engineered bacteria of the disclosure comprise an oxygen level-dependent promoter induced by low-oxygen, microaerobic, or anaerobic conditions. In one embodiment, the genetically engineered bacteria comprise a promoter induced by a molecule or metabolite, for example, a tissue-specific molecule or metabolite or a molecule or metabolite indicative of liver damage. In one embodiment, the metabolites may be gut specific. In one embodiment, the metabolite may be associated with hepatic encephalopathy, e.g., bilirubin. Non-limiting examples of molecules or metabolites include, e.g., bilirubin, aspartate aminotransferase, alanine aminotransferase, blood coagulation factors II, VII, IX, and X, alkaline phosphatase, gamma glutamyl transferase, hepatitis antigens and antibodies, alpha fetoprotein, anti-mitochondrial, smooth muscle, and anti-nuclear antibodies, iron, transferrin, ferritin, copper, ceruloplasmin, ammonia, and manganese in their blood and intestines. Promoters that respond to one of these molecules or their metabolites may be used in the genetically engineered bacteria provided herein. In one embodiment, the genetically engineered bacteria comprise a promoter induced by inflammation or an inflammatory response, e.g., RNS or ROS promoter. In one embodiment, the genetically engineered bacteria comprise a promoter induced by a metabolite that may or may not be naturally present (e.g., can be exogenously added) in the gut, e.g., arabinose and tetracycline.

“Exogenous environmental condition(s)” refer to setting(s) or circumstance(s) under which the promoter described herein is induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In one embodiment, the exogenous environmental conditions are specific to the gut of a mammal. In one embodiment, the exogenous environmental conditions are specific to the upper gastrointestinal tract of a mammal. In one embodiment, the exogenous environmental conditions are specific to the lower gastrointestinal tract of a mammal. In one embodiment, the exogenous environmental conditions are specific to the small intestine of a mammal. In one embodiment, exogenous environmental conditions refer to the presence of molecules or metabolites that are specific to the mammalian gut in a healthy or disease state (e.g., HE). In one embodiment, the exogenous environmental conditions are low-oxygen, microaerobic, or anaerobic conditions, such as the environment of the mammalian gut. In one embodiment, exogenous environmental conditions are molecules or metabolites that are specific to the mammalian gut, e.g., propionate. In one embodiment, the exogenous environmental condition is a tissue-specific or disease-specific metabolite or molecule(s). In one embodiment, the exogenous environmental condition is a low-pH environment. In one embodiment, the genetically engineered microorganism of the disclosure comprises a pH-dependent promoter. In one embodiment, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics.

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 3.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 3
Examples of transcription factors and
responsive genes and regulatory regions

| Transcription | Exemplary responsive genes, |
|---|---|
| Factor | promoters, and/or regulatory regions: |
| FNR | nirB, ydfZ, pdhR, focA, ndH, |
| | hlyE, narK, narX, narG, yfiD, tdcD |
| ANR | arcDABC |
| DNR | norb, norC |

As used herein, a “gene cassette” or “operon” encoding a biosynthetic pathway refers to the two or more genes that are required to produce a gut barrier function enhancer molecule, e.g., butyrate, propionate. In addition to encoding a set of genes capable of producing said molecule, the gene cassette or operon may also comprise additional transcription and translation elements, e.g., a ribosome binding site.

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In one embodiment, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In one embodiment, “non-native” refers to two or more nucleic acid sequences that are not found in the same relationship to each other in nature. The non-native nucleic acid sequence, e.g., gene or gene cassette, may be present on a plasmid or bacterial chromosome. In one embodiment, the genetically engineered bacteria of the disclosure comprise a gene cassette that is operably linked to a directly or indirectly inducible promoter that is not associated with said gene cassette in nature, e.g., a FNR-responsive promoter operably linked to an arginine production cassette. In addition, multiple copies of the gene, gene cassette, or regulatory region may be present in the bacterium, wherein one or more copies may be mutated or otherwise altered as described herein. In one embodiment, the genetically engineered bacteria are engineered to comprise multiple copies of the same non-native nucleic acid sequence, e.g., gene, gene cassette, or regulatory region, in order to enhance copy number or to comprise multiple different components of a gene cassette performing multiple different functions.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013) P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), a bacteriophage SP6 promoter (e.g., SP6 promoter (BBa_J64998)), and functional fragments thereof.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence. Furthermore, the term “gene” or “gene sequence” also refers to any sequence expressing a polypeptide or protein, including genomic sequences, cDNA sequences, naturally occurring sequences, artificial sequences, and codon optimized sequences. The term “gene” or “gene sequence” inter alia includes modification of endogenous genes, such as deletions, mutations, and expression of native and non-native genes under the control of a promoter that that they are not normally associated with in nature.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. In some aspects, the microorganism is engineered (“engineered microorganism” or “modified microorganism”) to produce one or more therapeutic molecules. In certain aspects, the microorganism is engineered to import and/or catabolize certain toxic metabolites, substrates, or other compounds from its environment, e.g., the gut. In certain aspects, the microorganism is engineered to synthesize certain beneficial metabolites, molecules, or other compounds (synthetic or naturally occurring) and release them into its environment. In certain embodiments, the engineered microorganism is an engineered bacterium. In certain embodiments, the engineered microorganism is an engineered virus.

As used herein, “payload” refers to one or more polynucleotides and/or polypeptides of interest to be produced by a genetically engineered microorganism, such as a bacteria or a virus. In one embodiment, the payload is encoded by a gene or multiple genes or an operon. In one embodiment, the one or more genes and/or operon(s) comprising the payload are endogenous to the microorganism. In one embodiment, the one or more elements of the payload is derived from a different microorganism and/or organism. In one embodiment, the payload is a therapeutic payload. In one embodiment, the payload is encoded by genes for the biosynthesis of a molecule. In one embodiment, the payload is encoded by genes for the metabolism, catabolism, or degradation of a molecule. In one embodiment, the payload is encoded by genes for the importation of a molecule. In one embodiment, the payload is encoded by genes for the exportation of a molecule. In one embodiment, the payload is a regulatory molecule(s), e.g., a transcriptional regulator such as FNR. In one embodiment, the payload comprises a regulatory element, such as a promoter or a repressor. In one embodiment, the payload comprises an inducible promoter, such as from FNRS. In some embodiments the payload comprises a repressor element, such as a kill switch. In alternate embodiments, the payload is produced by a biosynthetic or biochemical pathway, wherein the biosynthetic or biochemical pathway may optionally be endogenous to the microorganism. In one embodiment, the genetically engineered microorganism comprises two or more payloads. Non-limiting examples of payload(s) include one or more of the following: (1) ArgA^{fbr}, (2) mutated ArgR, (3) mutated ArgG. Other exemplary payloads include mutated sequence(s) that result in an auxotrophy, e.g., thyA auxotrophy, kill switch circuit, antibiotic resistance circuits, transporter sequence for importing biological molecules or substrates, secretion circuit.

“Probiotic” is used to refer to live, non-pathogenic microorganisms, e.g., bacteria, which can confer health benefits to a host organism that contains an appropriate amount of the microorganism. In one embodiment, the host organism is a mammal. In one embodiment, the host organism is a human. Some species, strains, and/or subtypes of non-pathogenic bacteria are currently recognized as probiotic bacteria. Examples of probiotic bacteria include, but are not limited to, Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stably maintained” or “stable” bacterium is used to refer to a bacterial host cell carrying non-native genetic material, e.g., a feedback resistant argA gene, mutant arginine repressor, and/or other mutant arginine regulon that is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a gene encoding an arginine racemase, in which the plasmid or chromosome carrying the arginine racemase gene is stably maintained in the bacterium, such that arginine racemase can be expressed in the bacterium, and the bacterium is capable of survival and/or growth in vitro and/or in vivo.

A “checkpoint inhibitor” refers to one or more therapeutic substances or drugs that are capable of reducing and/or inhibiting cell growth or replication. In one embodiment, the checkpoint inhibitor is a therapeutic molecule that is useful for modulating or treating a cancer. In one embodiment, the checkpoint inhibitor is a therapeutic molecule encoded by a gene. Non-limiting examples of checkpoint inhibitors include e.g., CTLA-4 antibodies, PD-1 antibodies, and PDL-1 antibodies.

“Gut” refers to the organs, glands, tracts, and systems that are responsible for the transfer and digestion of food, absorption of nutrients, and excretion of waste. In humans, the gut comprises the gastrointestinal tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

“Subject,” as used herein, means an animal. In one embodiment, the animal is a mammal, commonly a human.

In preferred embodiments, the combination therapies disclosed herein produce “synergistic therapeutic effect(s)” in the treatment of a disease, e.g. cancer. The term “synergistic” refers to effects that are greater than additive effects (e.g., greater efficacy) of each monotherapy in aggregate. A “therapeutic effect” encompasses any effect provided by any therapy that is sufficient to confer a benefit, e.g., clinical benefit.

As used herein a “pharmaceutical composition” refers to a preparation of genetically engineered bacteria of the disclosure with other components such as a physiologically suitable carrier and/or excipient.

The terms “therapeutically effective dose” and “therapeutically effective amount” are used to refer to an amount of a compound that results in prevention, delay of onset of symptoms, or amelioration of symptoms of a condition. A therapeutically effective amount may, for example, be sufficient to treat, prevent, reduce the severity, delay the onset, and/or reduce the risk of occurrence of one or more symptoms of a disorder associated with elevated ammonia concentrations. A therapeutically effective amount, as well as a therapeutically effective frequency of administration, can be determined by methods known in the art and discussed below.

As used herein, the term “partial regression” refers to an inhibition of growth of a tumor, and/or the regression of a tumor, e.g., in size, after administration of the bacterium and checkpoint inhibitor to a subject having the tumor. In one embodiment, a “partial regression” may refer to a regression of a tumor, e.g., in size, by at least about 5%, at least about 10%, at least about 15%, at least about 20%, at least about 25%, at least about 30%, at least about 40%, at least about 50%, at least about 60%, at least about 70%, at least about 80%, or at least about 90%. In another embodiment, a “partial regression” may refer to a decrease in the size of a tumor by at least about 5%, at least about 10%, at least about 15%, at least about 20%, at least about 25%, at least about 30%, at least about 40%, at least about 50%, at least about 60%, at least about 70%, at least about 75%, at least about 80%, or at least about 90%. In one embodiment, “partial regression” refers to the regression of a tumor, e.g., in size, but wherein the tumor is still detectable in the subject.

As used herein, the term “complete regression” refers to a complete regression of a tumor, e.g., in size, after administration of the bacteria and checkpoint inhibitor to the subject having the tumor. When “complete regression” occurs the tumor is undetectable in the subject.

As used herein, the term “percent response” refers to a percentage of subjects in a population of subjects who exhibit either a partial regression or a complete regression, as defined herein, after administration. For example, in one embodiment, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of subjects in a population of subjects exhibit a partial response or a complete response.

As used herein, the term “stable disease” refers to a cancer or tumor that is neither growing nor shrinking. “Stable disease” also refers to a disease state where no new tumors have developed, and a cancer or tumor has not spread to any new region or area of the body, e.g., by metastasis.

“Intratumoral administration” is meant to include any and all means for microorganism delivery to the intratumoral site and is not limited to intratumoral injection means. Examples of delivery means for the microorganisms is discussed in detail herein.

“Cancer” or “cancerous” is used to refer to a physiological condition that is characterized by unregulated cell growth. In one embodiment, cancer refers to a tumor. “Tumor” is used to refer to any neoplastic cell growth or proliferation or any pre-cancerous or cancerous cell or tissue. A tumor may be malignant or benign. Types of cancer include, but are not limited to, adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, liver cancer, lung cancer, malignant mesothelioma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macroglobulinemia, and Wilms tumor. Side effects of cancer treatment may include, but are not limited to, opportunistic autoimmune disorder(s), systemic toxicity, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration (National Cancer Institute).

As used herein, “abscopal” and “abscopal effect” refers to an effect in which localized treatment of a tumor not only shrinks or otherwise affects the tumor being treated, but also shrinks or otherwise affects other tumors outside the scope of the localized treatment. In one embodiment, the bacteria may elicit an abscopal effect. In one embodiment, no abscopal effect is observed upon administration of the bacteria.

In any of these embodiments in which abscopal effect is observed, timing of tumor growth in a tumor of the same type which is distal to the administration site is delayed by at least about 0 to 2 days, at least about 2 to 4 days, at least about 4 to 6 days, at least about 6 to 8 days, at least about 8 to 10 days, at least about 10 to 12 days, at least about 12 to 14 days, at least about 14 to 16 days, at least about 16 to 18 days, at least about 18 to 20 days, at least about 20 to 25 days, at least about 25 to 30 days, at least about 30 to 35 days of the same type relative to the tumor growth (tumor volume) in a naive animal or subject.

In any of these embodiments in which an abscopal effect is observed, timing of tumor growth as measured in tumor volume in a distal tumor of the same type is delayed by at least about 0 to 2 weeks, at least about 2 to 4 weeks, at least about 4 to 6 weeks, at least about 6 to 8 weeks, at least about 8 to 10 weeks, at least about 10 to 12 weeks, at least about 12 to 14 weeks, at least about 14 to 16 weeks, at least about 16 to 18 weeks, at least about 18 to 20 weeks, at least about 20 to 25 weeks, at least about 25 to 30 weeks, at least about 30 to 35 weeks, at least about 35 to 40 weeks, at least about 40 to 45 weeks, at least about 45 to 50 weeks, at least about 50 to 55 weeks, at least about 55 to 60 weeks, at least about 60 to 65 weeks, at least about 65 to 70 weeks, at least about 70 to 80 weeks, at least about 80 to 90 weeks, or at least about 90 to 100 in a tumor re-challenge relative to the tumor growth (tumor volume) in a naive animal or subject.

In any of these embodiments in which abscopal effect is observed, timing of tumor growth as measured in tumor volume in a tumor distal to the administration site of the same type is delayed by at least about 0 to 2 years, at least about 2 to 4 years, at least about 4 to 6 years, at least about 6 to 8 years, at least about 8 to 10 years, at least about 10 to 12 years, at least about 12 to 14 years, at least about 14 to 16 years, at least about 16 to 18 years, at least about 18 to 20 years, at least about 20 to 25 years, at least about 25 to 30 years, at least about 30 to 35 years, at least about 35 to 40 years, at least about 40 to 45 years, at least about 45 to 50 years, at least about 50 to 55 years, at least about 55 to 60 years, at least about 60 to 65 years, at least about 65 to 70 years, at least about 70 to 80 years, at least about 80 to 90 years, or at least about 90 to 100 in a tumor re-challenge relative to the tumor growth (tumor volume) in a naive animal or subject.

In yet another embodiment, survival rate is at least about 1.0-1.2-fold, at least about 1.2-1.4-fold, at least about 1.4-1.6-fold, at least about 1.6-1.8-fold, at least about 1.8-2-fold, or at least about two-fold greater in a tumor re-challenge as compared to the tumor growth (tumor volume) in a naive subject. In yet another embodiment, survival rate is at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold greater in a tumor re-challenge as compared to the tumor growth (tumor volume) in a naive subject. In this example, “tumor re-challenge” may also include metastasis formation which may occur in a subject at a certain stage of cancer progression.

Immunological memory represents an important aspect of the immune response in mammals. Memory responses form the basis for the effectiveness of vaccines against cancer cells. As used herein, the term “immune memory” or “immunological memory” refers to a state in which long-lived antigen-specific lymphocytes are available and are capable of rapidly mounting responses upon repeat exposure to a particular antigen. The importance of immunological memory in cancer immunotherapy is known, and the trafficking properties and long-lasting anti-tumor capacity of memory T cells play a crucial role in the control of malignant tumors and prevention of metastasis or reoccurrence. Immunological memory exists for both B lymphocytes and for T cells, and is now believed to exist in a large variety of other immune cells, including NK cells, macrophages, and monocytes. (see e.g., Farber et al., Immunological memory: lessons from the past and a look to the future (Nat. Rev. Immunol. (2016) 16: 124-128). Memory B cells are plasma cells that are able to produce antibodies for a long time. The memory B cell has already undergone clonal expansion and differentiation and affinity maturation, so it is able to divide multiple times faster and produce antibodies with much higher affinity. Memory T cells can be both CD4+ and CD8+. These memory T cells do not require further antigen stimulation to proliferate therefore they do not need a signal via MHC.

Immunological memory can, for example, be measured in an animal model by re-challenging the animal model upon achievement of complete regression upon treatment with the microorganism. The animal is then implanted with cancer cells from the cancer cell line and growth is monitored and compared to an age matched naïve animal of the same type which had not previously been exposed to the tumor. Such a tumor re-challenge is used to demonstrate systemic and long term immunity against tumor cells and may represent the ability to fight off future recurrence or metastasis formation. Such an experiment is described herein using the A20 tumor model in the Examples. Immunological memory would prevent or slow the reoccurrence of the tumor in the re-challenged animal relative to the naïve animal. On a cellular level, formation of immunological memory can be measured by expansion and/or persistence of tumor antigen specific memory or effector memory T cells.

In one embodiment, immunological memory is achieved in a subject upon administration of the compositions described herein. In one embodiment, immunological memory is achieved cancer patient upon administration of the compositions described herein.

In one embodiment, a complete response is achieved in a subject upon administration of the compositions described herein. In one embodiment, a complete response is achieved in a cancer patient upon administration.

In one embodiment, a complete remission is achieved in a subject upon administration of the compositions described herein. In one embodiment, a complete remission is achieved in a cancer patient upon administration.

In one embodiment, a partial response is achieved in a subject upon administration of the compositions described herein. In one embodiment, a partial response is achieved in a cancer patient upon administration of the compositions described herein.

In one embodiment, stable disease is achieved in a subject upon administration of the compositions described herein. In one embodiment, a partial response is achieved in a cancer patient upon administration of the compositions described herein.

In one embodiment, a subset of subjects within a group achieves a partial or complete response upon administration of the compositions described herein. In one embodiment, a subset of patients within a group achieve a partial or complete response upon administration of the compositions described herein.

In any of these embodiments in which immunological memory is observed, timing of tumor growth is delayed by at least about 0 to 2 days, at least about 2 to 4 days, at least about 4 to 6 days, at least about 6 to 8 days, at least about 8 to 10 days, at least about 10 to 12 days, at least about 12 to 14 days, at least about 14 to 16 days, at least about 16 to 18 days, at least about 18 to 20 days, at least about 20 to 25 days, at least about 25 to 30 days, at least about 30 to 35 days in a tumor re-challenge relative to the tumor growth (tumor volume) in a naive animal or subject.

In any of these embodiments in which immunological memory is observed, timing of tumor growth as measured in tumor volume delayed by at least about 0 to 2 weeks, at least about 2 to 4 weeks, at least about 4 to 6 weeks, at least about 6 to 8 weeks, at least about 8 to 10 weeks, at least about 10 to 12 weeks, at least about 12 to 14 weeks, at least about 14 to 16 weeks, at least about 16 to 18 weeks, at least about 18 to 20 weeks, at least about 20 to 25 weeks, at least about 25 to 30 weeks, at least about 30 to 35 weeks, at least about 35 to 40 weeks, at least about 40 to 45 weeks, at least about 45 to 50 weeks, at least about 50 to 55 weeks, at least about 55 to 60 weeks, at least about 60 to 65 weeks, at least about 65 to 70 weeks, at least about 70 to 80 weeks, at least about 80 to 90 weeks, or at least about 90 to 100 in a tumor re-challenge relative to the tumor growth (tumor volume) in a naive animal or subject.

In any of these embodiments in which immunological memory is observed, timing of tumor growth as measured in tumor volume delayed by at least about 0 to 2 years, at least about 2 to 4 years, at least about 4 to 6 years, at least about 6 to 8 years, at least about 8 to 10 years, at least about 10 to 12 years, at least about 12 to 14 years, at least about 14 to 16 years, at least about 16 to 18 years, at least about 18 to 20 years, at least about 20 to 25 years, at least about 25 to 30 years, at least about 30 to 35 years, at least about 35 to 40 years, at least about 40 to 45 years, at least about 45 to 50 years, at least about 50 to 55 years, at least about 55 to 60 years, at least about 60 to 65 years, at least about 65 to 70 years, at least about 70 to 80 years, at least about 80 to 90 years, or at least about 90 to 100 in a tumor re-challenge relative to the tumor growth (tumor volume) in a naive animal or subject.

In yet another embodiment, survival rate is at least about 1.0-1.2-fold, at least about 1.2-1.4-fold, at least about 1.4-1.6-fold, at least about 1.6-1.8-fold, at least about 1.8-2-fold, or at least about two-fold greater in a tumor re-challenge as compared to the tumor growth (tumor volume) in a naive subject. In yet another embodiment, survival rate is at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold greater in a tumor re-challenge as compared to the tumor growth (tumor volume) in a naive subject.

As used herein, “hot tumors” refer to tumors, which are T cell inflamed, i.e., associated with a high abundance of T cells infiltrating into the tumor. “Cold tumors” are characterized by the absence of effector T cells infiltrating the tumor and are further grouped into “immune excluded” tumors, in which immune cells are attracted to the tumor but cannot infiltrate the tumor microenvironment, and “immune ignored” phenotypes, in which no recruitment of immune cells occurs at all (further reviewed in Van der Woude et al., Migrating into the Tumor: a Roadmap for T Cells. Trends Cancer. 2017 November; 3(11):797-808).

“Hypoxia” is used to refer to reduced oxygen supply to a tissue as compared to physiological levels, thereby creating an oxygen-deficient environment. “Normoxia” refers to a physiological level of oxygen supply to a tissue. Hypoxia is a hallmark of solid tumors and characterized by regions of low oxygen and necrosis due to insufficient perfusion (Groot et al., 2007).

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere.

In one embodiment, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In one embodiment, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg O₂, 46.3 mmHg O₂, 58.75 mmHg, etc., which exemplary fractions are listed here for illustrative purposes and not meant to be limiting in any way). In one embodiment, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties.

In one embodiment, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In one embodiment, “low oxygen” is meant to refer to the level, amount, or concentration of oxygen (O₂) present in partially aerobic, semi aerobic, microaerobic, nonaerobic, microoxic, hypoxic, anoxic, and/or anaerobic conditions. In one embodiment, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂).

In one embodiment, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated.

In one embodiment, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.).

The exemplary fractions and ranges listed here are for illustrative purposes and not meant to be limiting in any way. In one embodiment, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current disclosure. A polypeptide of the disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the disclosure, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present disclosure include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr; Cys, Ser, Tyr, Thr; Val, Ile, Leu, Met, Ala, Phe; Lys, Arg, His; Phe, Tyr, Trp, His; and Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the disclosure. Such variants generally retain the functional activity of the peptides of the present disclosure. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism.

Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting the protein of interest or therapeutic protein from the microbial, e.g., bacterial cytoplasm. The secretion system may comprise a single protein or may comprise two or more proteins assembled in a complex e.g. HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In one embodiment, the protein(s) of interest or therapeutic protein(s) include a “secretion tag” of either RNA or peptide origin to direct the protein(s) of interest or therapeutic protein(s) to specific secretion systems. In one embodiment, the secretion system is able to remove this tag before secreting the protein(s) of interest or therapeutic protein(s) from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the protein(s) of interest or therapeutic protein(s) into the extracellular milieu.

As used herein, the term “transporter” is meant to refer to a mechanism, e.g., protein or proteins, for importing a molecule, e.g., amino acid, toxin, metabolite, substrate, etc. into the microorganism from the extracellular milieu.

“Bacteria for intratumoral administration” refer to bacteria that are capable of directing themselves to cancerous cells. Bacteria for intratumoral administration may be naturally capable of directing themselves to cancerous cells, necrotic tissues, and/or hypoxic tissues.

In one embodiment, bacteria that are not naturally capable of directing themselves to cancerous cells, necrotic tissues, and/or hypoxic tissues are genetically engineered to direct themselves to cancerous cells, necrotic tissues, and/or hypoxic tissues. Bacteria for intratumoral administration may be further engineered to enhance or improve desired biological properties, mitigate systemic toxicity, and/or ensure clinical safety. These species, strains, and/or subtypes may be attenuated, e.g., deleted for a toxin gene.

In one embodiment, bacteria for intratumoral administration have low infection capabilities. In one embodiment, bacteria for intratumoral administration are motile. In one embodiment, the bacteria for intratumoral administration are capable of penetrating deeply into the tumor, where standard treatments do not reach. In one embodiment, bacteria for intratumoral administration are capable of colonizing at least 20%, at least 30%, at least 40%, at least 50%, at least 60%, at least 70%, at least 80%, at least 90%, or at least 95% of a malignant tumor. Examples of bacteria for intratumoral administration include, but are not limited to, Bifidobacterium, Caulobacter, Clostridium, Escherichia coli, Listeria, Mycobacterium, Salmonella, Streptococcus, and Vibrio, e.g., Bifidobacterium adolescentis, Bifidobacterium bifidum, Bifidobacterium breve UCC2003, Bifidobacterium infantis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera (Cronin et al., 2012; Forbes, 2006; Jain and Forbes, 2001; Liu et al., 2014; Morrissey et al., 2010; Nuno et al., 2013; Patyar et al., 2010; Cronin, et al., Mol Ther 2010; 18:1397-407). In one embodiment, the bacteria for intratumoral administration are non-pathogenic bacteria. In one embodiment, intratumoral administration is done via injection.

“Microorganism” refers to an organism or microbe of microscopic, submicroscopic, or ultramicroscopic size that typically consists of a single cell. Examples of microorganisms include bacteria, viruses, parasites, fungi, certain algae, protozoa, and yeast. In some aspects, the microorganism is modified (“modified microorganism”) from its native state. In certain embodiments, the modified microorganism is a modified bacterium. In one embodiment, the modified microorganism is a genetically engineered bacterium. In certain embodiments, the modified microorganism is a modified yeast. In other embodiments, the modified microorganism is a genetically engineered yeast.

As used herein, the term “recombinant microorganism” refers to a microorganism, e.g., bacterial, yeast, or viral cell, or bacteria, yeast, or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial, yeast, or viral cell, or bacteria, yeast, or virus, that has been genetically modified from its native state to perform a specific function. Thus, a “programmed or bacterial cell” or “programmed or bacteria” refers to a bacterial cell or bacteria that has been genetically modified from its native state to perform a specific function. In certain embodiments, the programmed or bacterial cell has been modified to express one or more proteins, for example, one or more proteins that have a therapeutic activity or serve a therapeutic purpose. The programmed or bacterial cell may additionally have the ability to stop growing or to destroy itself once the protein(s) of interest have been expressed.

“Non-pathogenic bacteria” refer to bacteria that are not capable of causing disease or harmful responses in a host. In one embodiment, non-pathogenic bacteria are Gram-negative bacteria. In one embodiment, non-pathogenic bacteria are Gram-positive bacteria. In one embodiment, non-pathogenic bacteria do not contain lipopolysaccharides (LPS). In one embodiment, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infant's, Bifidobacterium lactis, Bifidobacterium longum, Clostridium butyricum, Enterococcus faecium, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976).

As used herein, the terms “modulate” and “treat” and their cognates refer to an amelioration of a cancer, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a cancer, either physically (e.g., stabilization of a discernible symptom), physiologically (e.g., stabilization of a physical parameter), or both. In another embodiment, “modulate” and “treat” refer to slowing the progression or reversing the progression of a cancer. As used herein, “prevent” and its cognates refer to delaying the onset or reducing the risk of acquiring a given cancer.

Those in need of treatment may include individuals already having a particular cancer, as well as those at risk of having, or who may ultimately acquire the cancer. The need for treatment is assessed, for example, by the presence of one or more risk factors associated with the development of a cancer (e.g., alcohol use, tobacco use, obesity, excessive exposure to ultraviolet radiation, high levels of estrogen, family history, genetic susceptibility), the presence or progression of a cancer, or likely receptiveness to treatment of a subject having the cancer. Cancer is caused by genomic instability and high mutation rates within affected cells. Treating cancer may encompass eliminating symptoms associated with the cancer and/or modulating the growth and/or volume of a subject's tumor, and does not necessarily encompass the elimination of the underlying cause of the cancer, e.g., an underlying genetic predisposition.

As used herein, the term “conventional cancer treatment” or “conventional cancer therapy” refers to treatment or therapy that is widely accepted and used by most healthcare professionals. It is different from alternative or complementary therapies, which are not as widely used. Examples of conventional treatment for cancer include surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, therapy, and blood product donation and transfusion.

As used herein “wild-type” refers to an unmodified bacteria. For example, a wild-type bacteria has not been modified using genetic engineering. A wild-type bacteria, for example, has not been modified to express a non-native gene or to comprise an auxotrophy. In one embodiment, a wild-type bacteria is an E. coli Nissle bacteria.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

### Bacteria

Disclosed herein are arginine-producing bacteria and/or ammonia-consuming bacteria, which can be administered in combination with a checkpoint inhibitor and surprisingly provide a striking synergistic effect on tumors. The arginine-producing bacteria and/or ammonia-consuming bacteria disclosed herein are capable of reducing excess ammonia and converting ammonia and/or nitrogen into alternate byproducts, such as arginine. In one embodiment, the bacteria are naturally non-pathogenic bacteria. In one embodiment, the bacteria are commensal bacteria. In one embodiment, the bacteria are probiotic bacteria. In one embodiment, the bacteria are naturally pathogenic bacteria that are modified or mutated to reduce or eliminate pathogenicity. Exemplary bacteria are described in U.S. Patent Provisional 62/757,452; US Patent Publication US20160333326; and International Patent Publications WO2017139697, WO2017123675, WO2018129404, WO2018012698, and WO2019014391, the contents of which are herein incorporated by reference in their entirety.

In certain embodiments, the bacteria are obligate anaerobic bacteria. In certain embodiments, the bacteria are facultative anaerobic bacteria. In certain embodiments, the bacteria are aerobic bacteria. In one embodiment, the bacteria are Gram-positive bacteria and lack LPS. In one embodiment, the bacteria are Gram-negative bacteria. In one embodiment, the bacteria are Gram-positive and obligate anaerobic bacteria. In one embodiment, the bacteria are Gram-positive and facultative anaerobic bacteria. In one embodiment, the bacteria are non-pathogenic bacteria. In one embodiment, the bacteria are commensal bacteria. In one embodiment, the bacteria are probiotic bacteria.

Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, Vibrio cholera, and the bacteria shown in Table 4. In certain embodiments, the bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the bacteria are selected from the group consisting of Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli Nissle, Lactobacillus acidophilus, Lactobacillus plantarum, Lactobacillus reuteri, and Lactococcus lactis.

In one embodiment, the bacteria are Gram-negative bacteria. In one embodiment, the bacteria are E. coli. For example, E. coli Nissle has been shown to preferentially colonize tumor tissue in vivo following either oral or intravenous administration (Zhang et al., 2012 and Danino et al., 2015). E. coli have also been shown to exhibit robust tumor-specific replication (Yu et al., 2008). In one embodiment, the bacteria are Escherichia coli strain Nissle 1917 (E. coli Nissle), a Gram-negative acterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added).

In one embodiment, Lactobacillus is used. Lactobacillus casei injected intravenously has been found to accumulate in tumors, which was enhanced through nitroglycerin (NG), a commonly used NO donor, likely due to the role of NO in increasing the blood flow to hypovascular tumors (Fang et al., 2016 (Methods Mol Biol. 2016; 1409:9-23 Enhancement of Tumor-Targeted Delivery of Bacteria with Nitroglycerin Involving Augmentation of the EPR Effect).

In one embodiment, the bacteria are obligate anaerobes. In one embodiment, the bacteria are Clostridia. Clostridia are obligate anaerobic bacterium that produce spores and are naturally capable of colonizing and in some cases lysing hypoxic tumors (Groot et al., 2007). In experimental models, Clostridia have been used to deliver pro-drug converting enzymes and enhance radiotherapy (Groot et al., 2007). In one embodiment, the bacteria is selected from the group consisting of Clostridium novyi-NT, Clostridium histolyticium, Clostridium tetani, Clostridium oncolyticum, Clostridium sporogenes, and Clostridium beijerinckii (Liu et al., 2014). In one embodiment, the Clostridium is naturally non-pathogenic. For example, Clostridium oncolyticum is a pathogenic and capable of lysing tumor cells. In alternate embodiments, the Clostridium is naturally pathogenic but modified to reduce or eliminate pathogenicity. For example, Clostridium novyi are naturally pathogenic, and Clostridium novyi-NT are modified to remove lethal toxins. Clostridium novyi-NT and Clostridium sporogenes have been used to deliver single-chain HIF-1α antibodies to treat cancer and is an “excellent tumor colonizing Clostridium strains” (Groot et al., 2007).

In one embodiment, the bacteria facultative anaerobes. In one embodiment, the bacteria are Salmonella, e.g., Salmonella typhimurium. Salmonella are non-spore-forming Gram-negative bacteria that are facultative anaerobes. In one embodiment, the Salmonella are naturally pathogenic but modified to reduce or eliminate pathogenicity. For example, Salmonella typhimurium is modified to remove pathogenic sites (attenuated). In one embodiment, the bacteria are Bifidobacterium. Bifidobacterium are Gram-positive, branched anaerobic bacteria. In one embodiment, the Bifidobacterium is naturally non-pathogenic. In alternate embodiments, the Bifidobacterium is naturally pathogenic but modified to reduce or eliminate pathogenicity. Bifidobacterium and Salmonella have been shown to preferentially target and replicate in the hypoxic and necrotic regions of tumors (Yu et al., 2014).

The bacteria may be administered systemically, orally, locally and/or intratumorally. In one embodiment, the bacteria are capable of targeting cancerous cells, particularly in the hypoxic regions of a tumor, and are administered in combination with, e.g., a checkpoint inhibitor provided herein.

In one embodiment, the bacterium is naturally capable of directing itself to cancerous cells, necrotic tissues, and/or hypoxic tissues. For example, bacterial colonization of tumors may be achieved without any specific genetic modifications in the bacteria or in the host (Yu et al., 2008). In one embodiment, the tumor-targeting bacterium is a bacterium that is not naturally capable of directing itself to cancerous cells, necrotic tissues, and/or hypoxic tissues, but is genetically engineered to do so. In one embodiment, the bacteria spread hematogenously to reach the targeted tumor(s). Bacterial infection has been linked to tumor regression (Hall, 1998; Nauts and McLaren, 1990), and certain bacterial species have been shown to localize to and lyse necrotic mammalian tumors (Jain and Forbes, 2001). Non-limiting examples of tumor-targeting bacteria are shown in Table 4.

TABLE 4
Bacteria with tumor-targeting capability

| Bacterial Strain | Reference |
|---|---|
| Clostridium novyi-NT | Forbes, Neil S. “Profile of a bacterial tumor killer.” |
| | Nature biotechnology 24.12 (2006): 1484-1485. |
| Bifidobacterium spp | Liu, Sai, et al. “Tumor-targeting bacterial therapy: |
| Streptococcus spp | A potential treatment for oral cancer.” |
| Caulobacter spp | Oncology letters 8.6 (2014): 2359-2366. |
| Clostridium spp | |
| Escherichia coli MG1655 | Cronin, Michelle, et al. “High resolution in vivo |
| Escherichia coli Nissle | bioluminescent imaging for the study of bacterial tumour |
| Bifidobacterium breve UCC2003 | targeting.” PloS one 7.1 (2012): e30940.; Zhou, et al., Med |
| Salmonella typhimurium | Hypotheses. 2011 Apr;76(4):533-4. doi: |
| | 10.1016/j.mehy.2010.12.010. Epub 2011 Jan 21; Zhang et al., |
| | Appl Environ Microbiol. 2012 Nov; 78(21): 7603-7610; |
| | Danino et al., Science Translational Medicine, |
| | 2015 Vol 7 Issue 289, pp. 289ra84 |
| Clostridium novyi-NT | Bernardes, Nuno, Ananda M. Chakrabarty, and |
| Bifidobacterium spp | Arsenio M. Fialho. “Engineering of bacterial |
| Mycobacterium bovis | strains and their products for cancer therapy.” |
| Listeria monocytogenes | Applied microbiology and biotechnology |
| Escherichia coli | 97.12 (2013): 5189-5199. |
| Salmonella spp | |
| Salmonella typhimurium | |
| Salmonella choleraesuis | Patyar, S., et al. “Bacteria in cancer therapy: |
| Vibrio cholera | a novel experimental strategy.” |
| Listeria monocytogenes | J Biomed Sci 17.1 (2010): 21-30. |
| Escherichia coli | |
| Bifidobacterium adolescentis | |
| Clostridium acetobutylicum | |
| Salmonella typhimurium | |
| Clostridium histolyticum | |
| Escherichia coli Nissle 1917 | Danino et al. “Programmable probiotics for |
| | detection of cancer in urine.” Sci Transl Med. |
| | 2015 May 27;7(289):289ra84 |

In certain aspects, the bacteria are able to selectively home to tumor microenvironments (TME). Thus, in certain embodiments, the bacteria can be administered systemically, e.g., via oral administration, intravenous injection, subcutaneous injection, or other means, and are able to selectively colonize a tumor site. For example, E. coli Nissle 1917 has been shown to selectively home into tumor tissue in rodent models of liver metastasis following oral delivery, but does not colonize healthy organs or fibrotic liver tissue. (Danino et al, 2015; Stritzker et al., Int J Med Micro, 297:151-162 (2007)). In other embodiments, the genetically engineered bacteria, such as a bacteria or virus, are delivered locally (directly) to the tumor site or microenvironment, e.g., via intratumoral administration, such as intratumoral injection.

In one embodiment, the bacteria of the disclosure proliferate and colonize a tumor. In one embodiment, colonization persists for several days, several weeks, several months, several years or indefinitely. In one embodiment, the bacteria do not proliferate in the tumor and bacterial counts drop off quickly post injection, e.g., less than a week post injection, until no longer detectable.

In one embodiment, the bacteria are administered repeatedly. In one embodiment, the bacteria are administered once.

In one embodiment, such bacteria are further mutated to attenuate one or more virulence factors. Other bacteria are described at least in Song et al., Infectious Agents and Cancer, 2018; and Lukasiewicz and Fol, J. Immunol. Research, 2018, Article ID 2397808.

Arginine and Ammonia

Specifically disclosed herein are arginine-producing bacteria and/or ammonia-consuming bacteria. In one embodiment, the arginine-producing bacteria are bacteria that have been modified, or genetically engineered, to consume ammonia. In one embodiment, the arginine-producing bacteria are bacteria that have been modified, or genetically engineered, to overproduce arginine. In one embodiment, the arginine-producing bacteria are bacteria that have been modified, or genetically engineered, to consume ammonia and to overproduce arginine.

In bacteria, e.g., Escherichia coli (E. coli), arginine biosynthesis converts glutamate to arginine in an eight-step enzymatic process involving the enzymes N-acetylglutamate synthetase, N-acetylglutamate kinase, N-acetylglutamate phosphate reductase, acetylornithine aminotransferase, N-acetylornithinase, carbamoylphosphate synthase, ornithine transcarbamylase, argininosuccinate synthase, and argininosuccinate lyase (Cunin et al., 1986). The first five steps involve N-acetylation to generate an ornithine precursor. The additional three steps involve carbamoylphosphate utilization to generate arginine. In some bacteria, e.g., Bacillus stearothermophilus, the first and fifth steps in arginine biosynthesis may be catalyzed by the bifunctional enzyme ornithine acetyltransferase. All of the genes encoding these enzymes are subject to repression by arginine via its interaction with the arginine repressor (ArgR) to form a complex that binds to the regulatory region of each gene and inhibits transcription. N-acetylglutamate synthetase is also subject to allosteric feedback inhibition at the protein level by arginine alone (Tuchman et al., 1997; Caldara et al., 2006; Caldara et al., 2008; Caldovic et al., 2010).

The genes that regulate arginine biosynthesis in bacteria are scattered across the chromosome and organized into multiple operons that are controlled by a single repressor, which Maas and Clark (1964) termed a “regulon.” Each operon is regulated by a regulatory region comprising at least one 18-nucleotide imperfect palindromic sequence, called an ARG box, that overlaps with the promoter (Tian et al., 1992; Tian et al., 1994). The argR gene encodes the repressor protein, which binds to one or more ARG boxes when associated with arginine (Lim et al., 1987). The ARG boxes that regulate each operon may be non-identical, and the consensus ARG box sequence is A/T nTGAAT A/T A/T T/A T/A ATTCAn T/A (SEQ ID NO:39; Maas, 1994). In addition, the regulatory region of argR contains two promoters, one of which overlaps with two ARG boxes and is autoregulated.

ArgA encodes N-acetylglutamate synthetase, argB encodes N-acetylglutamate kinase, argC encodes N-acetylglutamylphosphate reductase, argD encodes acetylornithine aminotransferase, argE encodes N-acetylornithinase, argF encodes ornithine transcarbamylase, argI also encodes ornithine transcarbamylase, argG encodes arginosuccinate synthase, argH encodes argininosuccinate lyase, and argJ encodes ornithine acetyltransferase. CarA encodes the small A subunit of carbamylphosphate synthase having glutaminase activity, and carB encodes the large B subunit of carbamylphosphate synthase that catalyzes carbamoylphosphate synthesis from ammonia. Different combinations of one or more of these arginine biosynthesis genes (i.e., argA, argB, argC, argD, argE, argF, argG, argH, argI, argJ, carA, and carB) may be organized, naturally or synthetically, into one or more operons, and such organization may vary between bacterial species, strains, and subtypes. Each operon is subject to arginine-mediated repression via ArgR.

In one embodiment, the arginine-producing bacteria may encode an arginine feedback resistant N-acetylglutamate synthetase, lack any functional ArgR repressor, and therefore ArgR repressor-mediated transcriptional repression of each of the arginine biosynthesis operons is reduced or eliminated. In one embodiment, each copy of a functional argR gene normally present in a corresponding wild-type bacterium is independently deleted or rendered inactive by one or more nucleotide deletions, insertions, or substitutions.

In one embodiment, each copy of a functional argR gene normally present in a corresponding wild-type bacterium is deleted. The genetically engineered bacteria lacking any functional ArgR repressor produce more arginine when the arginine feedback resistant argA gene is expressed than unmodified bacteria of the same bacterial subtype under the same conditions. Enhancement of arginine biosynthesis may be used to incorporate excess nitrogen in the body into non-toxic molecules for cancer immunotherapy.

The genetically engineered bacteria comprise an arginine feedback resistant N-acetylglutamate synthetase protein (argA^{fbr}) that is significantly “less sensitive to L-arginine than the enzyme from the feedback sensitive parent strain” (see, e.g., Eckhardt et al., 1975; Rajagopal et al., 1998). The feedback resistant argA gene is present on a plasmid or chromosome. In one embodiment, expression from the plasmid may be useful for increasing argA^{fbr} expression. In one embodiment, expression from the chromosome may be useful for increasing stability of argA^{fbr} expression.

Multiple distinct feedback resistant N-acetylglutamate synthetase proteins are known in the art and may be combined in the genetically engineered bacteria. In one embodiment, the argA^{fbr} gene is expressed under the control of a constitutive promoter. In one embodiment, the argA^{fbr} gene is expressed under the control of a promoter that is induced by exogenous environmental conditions. In one embodiment, the argA^{fbr} gene is under control of an oxygen level-dependent promoter. In a more specific aspect, the argA^{fbr} gene is under control of an oxygen level-dependent promoter that is activated under low-oxygen or anaerobic environments, such as a tumor micro-environment.

In certain embodiments, the genetically engineered bacteria comprise argA^{fbr} expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. In one embodiment, multiple distinct FNR nucleic acid sequences are inserted in the genetically engineered bacteria. In alternate embodiments, the genetically engineered bacteria comprise argA^{fbr} expressed under the control of an alternate oxygen level-dependent promoter, e.g., an anaerobic regulation of arginine deiminase and nitrate reduction ANR promoter (Ray et al., 1997), a dissimilatory nitrate respiration regulator DNR promoter (Trunk et al., 2010). In these embodiments, arginine biosynthesis is particularly activated in a low-oxygen or anaerobic environment, such as such as a tumor micro-environment.

In P. aeruginosa, the anaerobic regulation of arginine deiminase and nitrate reduction (ANR) transcriptional regulator is “required for the expression of physiological functions which are inducible under oxygen-limiting or anaerobic conditions” (Winteler et al., 1996; Sawers 1991). P. aeruginosa ANR is homologous with E. coli FNR, and “the consensus FNR site (TTGAT----ATCAA) (SEQ ID NO:40) was recognized efficiently by ANR and FNR” (Winteler et al., 1996). Like FNR, in the anaerobic state, ANR activates numerous genes responsible for adapting to anaerobic growth. In the aerobic state, ANR is inactive. Pseudomonas fluorescens, Pseudomonas putida, Pseudomonas syringae, and Pseudomonas mendocina all have functional analogs of ANR (Zimmermann et al., 1991). Promoters that are regulated by ANR are known in the art, e.g., the promoter of the arcDABC operon (see, e.g., Hasegawa et al., 1998).

The FNR family also includes the dissimilatory nitrate respiration regulator (DNR) (Arai et al., 1995), a transcriptional regulator which is required in conjunction with ANR for “anaerobic nitrate respiration of Pseudomonas aeruginosa” (Hasegawa et al., 1998). For certain genes, the FNR-binding motifs “are probably recognized only by DNR” (Hasegawa et al., 1998). Any suitable transcriptional regulator that is controlled by exogenous environmental conditions and corresponding regulatory region may be used. Non-limiting examples include ArcA/B, ResD/E, NreA/B/C, and AirSR, and others are known in the art.

FNR promoter sequences are known in the art, and any suitable FNR promoter sequence(s) may be used in the genetically engineered bacteria of the invention. Any suitable FNR promoter(s) may be combined with any suitable argA^{fbr}(e.g., the exemplary argA^{fbr} sequence shown in Table 6). Non-limiting FNR promoter sequences are provided in Table 5. Table 5 depicts the nucleic acid sequences of exemplary regulatory region sequences comprising a FNR-responsive promoter sequence. Underlined sequences are predicted ribosome binding sites, and bolded sequences are restriction sites used for cloning. In some embodiments, the genetically engineered bacteria of the invention comprise one or more of: SEQ ID NO: 18, SEQ ID NO: 19, nirB1 promoter (SEQ ID NO: 20), nirB2 promoter (SEQ ID NO: 21), nirB3 promoter (SEQ ID NO: 22), ydfZ promoter (SEQ ID NO: 23), nirB promoter fused to a strong ribosome binding site (SEQ ID NO: 24), ydfZ promoter fused to a strong ribosome binding site (SEQ ID NO: 25), fnrS, an anaerobically induced small RNA gene (fnrS1 promoter SEQ ID NO: 26 or fnrS2 promoter SEQ ID NO: 27), nirB promoter fused to a crp binding site (SEQ ID NO: 28), and fnrS fused to a crp binding site (SEQ ID NO: 29).

In some embodiments, genetically engineered bacteria comprise a nucleic acid sequence that is at least about 80%, at least about 85%, at least about 90%, at least about 95%, or at least about 99% homologous to the DNA sequence of SEQ ID NO: 18, 19, 20, 21, 22, 23, 24, 25, 26, 27, 28, or 29, or a functional fragment thereof

TABLE 5

| FNR-responsive | |
|---|---|
| regulatory region | SEQUENCE |
| SEQ ID NO: 18 | ATCCCCATCACTCTTGATGGAGATCAATTCCCCAAGCTGCTAGAGCGTTACCTTGCCCTT |
| | AAACATTAGCAATGTCGATTTATCAGAGGGCCGACAGGCTCCCACAGGAGAAAACCG |
| SEQ ID NO: 19 | CTCTTGATCGTTATCAATTCCCACGCTGTTTCAGAGCGTTACCTTGCCCTTAAACATTAG |
| | CAATGTCGATTTATCAGAGGGCCGACAGGCTCCCACAGGAGAAAACCG |
| nirB1 | GTCAGCATAACACCCTGACCTCTCATTAATTGTTCATGCCGGGCGGCACTATCGTCGTCC |
| SEQ ID NO: 20 | GGCCTTTTCCTCTCTTACTCTGCTACGTACATCTATTTCTATAAATCCGTTCAATTTGTC |
| | TGTTTTTTGCACAAACATGAAATATCAGACAATTCCGTGACTTAAGAAAATTTATACAAA |
| | TCAGCAATATACCCCTTAAGGAGTATATAAAGGTGAATTTGATTTACATCAATAAGCGGG |
| | GTTGCTGAATCGTTAAGGTAGGCGGTAATAGAAAAGAAATCGAGGCAAAA |
| nirB2 | CGGCCCGATCGTTGAACATAGCGGTCCGCAGGCGGCACTGCTTACAGCAAACGGTCTGTA |
| SEQ ID NO: 21 | CGCTGTCGTCTTTGTGATGTGCTTCCTGTTAGGTTTCGTCAGCCGTCACCGTCAGCATAA |
| | CACCCTGACCTCTCATTAATTGCTCATGCCGGACGGCACTATCGTCGTCCGGCCTTTTCC |
| | TCTCTTCCCCCGCTACGTGCATCTATTTCTATAAACCCGCTCATTTTGTCTATTTTTTGC |
| | ACAAACATGAAATATCAGACAATTCCGTGACTTAAGAAAATTTATACAAATCAGCAATAT |
| | ACCCATTAAGGAGTATATAAAGGTGAATTTGATTTACATCAATAAGCGGGGTTGCTGAAT |
| | CGTTAAGGTAGGCGGTAATAGAAAAGAAATCGAGGCAAAAatgtttgtttaactttaaga |
| | aggagatatacat |
| nirB3 | GTCAGCATAACACCCTGACCTCTCATTAATTGCTCATGCCGGACGGCACTATCGTCGTCC |
| SEQ ID NO: 22 | GGCCTTTTCCTCTCTTCCCCCGCTACGTGCATCTATTTCTATAAACCCGCTCATTTTGTC |
| | TATTTTTTGCACAAACATGAAATATCAGACAATTCCGTGACTTAAGAAAATTTATACAAA |
| | TCAGCAATATACCCATTAAGGAGTATATAAAGGTGAATTTGATTTACATCAATAAGCGGG |
| | GTTGCTGAATCGTTAAGGTAGGCGGTAATAGAAAAGAAATCGAGGCAAAA |
| ydfZ | ATTTCCTCTCATCCCATCCGGGGTGAGAGTCTTTTCCCCCGACTTATGGCTCATGCATGC |
| SEQ ID NO: 23 | ATCAAAAAAGATGTGAGCTTGATCAAAAACAAAAAATATTTCACTCGACAGGAGTATTTA |
| | TATTGCGCCCGTTACGTGGGCTTCGACTGTAAATCAGAAAGGAGAAAACACCT |
| nirB + RBS | GTCAGCATAACACCCTGACCTCTCATTAATTGTTCATGCCGGGCGGCACTATCGTCGTCC |
| SEQ ID NO: 24 | GGCCTTTTCCTCTCTTACTCTGCTACGTACATCTATTTCTATAAATCCGTTCAATTTGTC |
| | TGTTTTTTGCACAAACATGAAATATCAGACAATTCCGTGACTTAAGAAAATTTATACAAA |
| | TCAGCAATATACCCCTTAAGGAGTATATAAAGGTGAATTTGATTTACATCAATAAGCGGG |
| | GTTGCTGAATCGTTAAGGATCCCTCTAGAAATAATTTTGTTTAACTTTAAGAAGGAGATA |
| | TACAT |
| ydfZ + RBS | CATTTCCTCTCATCCCATCCGGGGTGAGAGTCTTTTCCCCCGACTTATGGCTCATGCATG |
| SEQ ID NO: 25 | CATCAAAAAAGATGTGAGCTTGATCAAAAACAAAAAATATTTCACTCGACAGGAGTATTT |
| | ATATTGCGCCCGGATCCCTCTAGAAATAATTTTGTTTAACTTTAAGAAGGAGATATACAT |
| fnrS1 | AGTTGTTCTTATTGGTGGTGTTGCTTTATGGTTGCATCGTAGTAAATGGTTGTAACAAAA |
| SEQ ID NO: 26 | GCAATTTTTCCGGCTGTCTGTATACAAAAACGCCGTAAAGTTTGAGCGAAGTCAATAAAC |
| | TCTCTACCCATTCAGGGCAATATCTCTCTTGGATCCCTCTAGAAATAATTTTGTTTAACT |
| | TTAAGAAGGAGATATACAT |
| fnrS2 | AGTTGTTCTTATTGGTGGTGTTGCTTTATGGTTGCATCGTAGTAAATGGTTGTAACAAAA |
| SEQ ID NO: 27 | GCAATTTTTCCGGCTGTCTGTATACAAAAACGCCGCAAAGTTTGAGCGAAGTCAATAAAC |
| | TCTCTACCCATTCAGGGCAATATCTCTCTTGGATCCAAAGTGAACTCTAGAAATAATTTT |
| | GTTTAACTTTAAGAAGGAGATATACAT |
| nirB + crp | TCGTCTTTGTGATGTGCTTCCTGTTAGGTTTCGTCAGCCGTCACCGTCAGCATAACACCC |
| SEQ ID NO: 28 | TGACCTCTCATTAATTGCTCATGCCGGACGGCACTATCGTCGTCCGGCCTTTTCCTCTCT |
| | TCCCCCGCTACGTGCATCTATTTCTATAAACCCGCTCATTTTGTCTATTTTTTGCACAAA |
| | CATGAAATATCAGACAATTCCGTGACTTAAGAAAATTTATACAAATCAGCAATATACCCA |
| | TTAAGGAGTATATAAAGGTGAATTTGATTTACATCAATAAGCGGGGTTGCTGAATCGTTA |
| | AGGTAGaaatgtgatctagttcacatttGCGGTAATAGAAAAGAAATCGAGGCAAAAatg |
| | tttgtttaactttaagaaggagatatacat |
| fnrS + crp | AGTTGTTCTTATTGGTGGTGTTGCTTTATGGTTGCATCGTAGTAAATGGTTGTAACAAAA |
| SEQ ID NO: 29 | GCAATTTTTCCGGCTGTCTGTATACAAAAACGCCGCAAAGTTTGAGCGAAGTCAATAAAC |
| | TCTCTACCCATTCAGGGCAATATCTCTCaaatgtgatctagttcacattttttgtttaac |
| | tttaagaaggagatatacat |

In one embodiment, the argA r gene is expressed under the control of a promoter that is induced by exposure to tetracycline. In one embodiment, gene expression is further optimized by methods known in the art, e.g., by optimizing ribosomal binding sites and/or increasing mRNA stability. The nucleic acid sequence of an exemplary argA^{fbr} sequence is shown in Table 6. The polypeptide sequence of an exemplary argA^{fbr} sequence is shown in Table 7.

TABLE 6
Nucleotide sequence of exemplary argA^{fbr} sequence
(SEQ ID NO: 30)

ATGGTAAAGGAACGTAAAACCGAGTTGGTCGAGGGATTCCGCCATTCGG
TTCCCTGTATCAATACCCACCGGGGAAAAACGTTTGTCATCATGCTCGG
CGGTGAAGCCATTGAGCATGAGAATTTCTCCAGTATCGTTAATGATATC
GGGTTGTTGCACAGCCTCGGCATCCGTCTGGTGGTGGTCTATGGCGCAC
GTCCGCAGATCGACGCAAATCTGGCTGCGCATCACCACGAACCGCTGTA
TCACAAGAATATACGTGTGACCGACGCCAAAACACTGGAACTGGTGAAG
CAGGCTGCGGGAACATTGCAACTGGATATTACTGCTCGCCTGTCGATGA
GTCTCAATAACACGCCGCTGCAGGGCGCGCATATCAACGTCGTCAGTGG
CAATTTTATTATTGCCCAGCCGCTGGGCGTCGATGACGGCGTGGATTAC
TGCCATAGCGGGCGTATCCGGCGGATTGATGAAGACGCGATCCATCGTC
AACTGGACAGCGGTGCAATAGTGCTAATGGGGCCGGTCGCTGTTTCAGT
CACTGGCGAGAGCTTTAACCTGACCTCGGAAGAGATTGCCACTCAACTG
GCCATCAAACTGAAAGCTGAAAAGATGATTGGTTTTTGCTCTTCCCAGG
GCGTCACTAATGACGACGGTGATATTGTCTCCGAACTTTTCCCTAACGA
AGCGCAAGCGCGGGTAGAAGCCCAGGAAGAGAAAGGCGATTACAACTCC
GGTACGGTGCGCTTTTTGCGTGGCGCAGTGAAAGCCTGCCGCAGCGGCG
TGCGTCGCTGTCATTTAATCAGTTATCAGGAAGATGGCGCGCTGTTGCA
AGAGTTGTTCTCACGCGACGGTATCGGTACGCAGATTGTGATGGAAAGC
GCCGAGCAGATTCGTCGCGCAACAATCAACGATATTGGCGGTATTCTGG
AGTTGATTCGCCCACTGGAGCAGCAAGGTATTCTGGTACGCCGTTCTCG
CGAGCAGCTGGAGATGGAAATCGACAAATTCACCATTATTCAGCGCGAT
AACACGACTATTGCCTGCGCCGCGCTCTATCCGTTCCCGGAAGAGAAGA
TTGGGGAAATGGCCTGTGTGGCAGTTCACCCGGATTACCGCAGTTCATC
AAGGGGTGAAGTTCTGCTGGAACGCATTGCCGCTCAGGCTAAGCAGAGC
GGCTTAAGCAAATTGTTTGTGCTGACCACGCGCAGTATTCACTGGTTCC
AGGAACGTGGATTTACCCCAGTGGATATTGATTTACTGCCCGAGAGCAA
AAAGCAGTTGTACAACTACCAGCGTAAATCCAAAGTGTTGATGGCGGAT
TTAGGGTAA

TABLE 7
Polypeptide sequence of exemplary argA^{fbr} sequence
(SEQ ID NO: 31)

MVKERKTELVEGFRHSVPCINTHRGKTFVIMLGGEAIEHENFSSIVNDIG
LLHSLGIRLVVVYGARPQIDANLAAHHHEPLYHKNIRVTDAKTLELVKQA
AGTLQLDITARLSMSLNNTPLQGAHINVVSGNFIIAQPLGVDDGVDYCHS
GRIRRIDEDAIHRQLDSGAIVLMGPVAVSVTGESFNLTSEEIATQLAIKL
KAEKMIGFCSSQGVTNDDGDIVSELFPNEAQARVEAQEEKGDYNSGTVRF
LRGAVKACRSGVRRCHLISYQEDGALLQELFSRDGIGTQIVMESAEQIRR
ATINDIGGILELIRPLEQQGILVRRSREQLEMEIDKFTIIQRDNTTIACA
ALYPFPEEKIGEMACVAVHPDYRSSSRGEVLLERIAAQAKQSGLSKLFVL
TTRSIHWFQERGETPVDIDLLPESKKQLYNYQRKSKVLMADLG
Bold underline: mutated amino acid resulting feedback resistance (mutation is Y19C)

In some embodiments, the genetically engineered bacteria comprise the nucleic acid sequence of SEQ ID NO: 30 or a functional fragment thereof. In some embodiments, the genetically engineered bacteria comprise a nucleic acid sequence that, but for the redundancy of the genetic code, encodes the same polypeptide as SEQ ID NO: 30 or a functional fragment thereof. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 30 or a functional fragment thereof, or a nucleic acid sequence that, but for the redundancy of the genetic code, encodes the same polypeptide as SEQ ID NO: 30 or a functional fragment thereof.

In some embodiments, the genetically engineered bacteria encode a polypeptide sequence of SEQ ID NO: 31 or a functional fragment thereof. In some embodiments, the genetically engineered bacteria encode a polypeptide sequence encodes a polypeptide, which contains one or more conservative amino acid substitutions relative to SEQ ID NO: 31 or a functional fragment thereof. In some embodiments, genetically engineered bacteria encode a polypeptide sequence that is at least about 80%, at least about 85%, at least about 90%, at least about 95%, or at least about 99% homologous to the DNA sequence of SEQ ID NO: 31 or a functional fragment thereof.

In one embodiment, arginine feedback inhibition of N-acetylglutamate synthetase is reduced by at least about 50%, at least about 60%, at least about 70%, at least about 80%, at least about 90%, or at least about 95% in the genetically engineered bacteria when the arginine feedback resistant N-acetylglutamate synthetase is active, as compared to a wild-type N-acetylglutamate synthetase from bacteria of the same subtype under the same conditions.

The genetically engineered bacteria may comprise a stably maintained plasmid or chromosome carrying the argA^{fbr} gene, such that argA^{fbr} can be expressed in the host cell, and the host cell is capable of survival and/or growth in vitro, e.g., in medium, and/or in vivo, e.g., in the gut. In one embodiment, a bacterium may comprise multiple copies of the feedback resistant argA gene. In one embodiment, the feedback resistant argA gene is expressed on a low-copy plasmid. In one embodiment, the low-copy plasmid may be useful for increasing stability of expression. In one embodiment, the low-copy plasmid may be useful for decreasing leaky expression under non-inducing conditions. In one embodiment, the feedback resistant argA gene is expressed on a high-copy plasmid. In one embodiment, the high-copy plasmid may be useful for increasing argA^{fbr} expression. In one embodiment, the plasmid also comprises wild-type ArgR binding sites, e.g., ARG boxes. In one embodiment, the plasmid does not comprise functional ArgR binding sites, e.g., the plasmid comprises modified ARG boxes, or the plasmid does not comprise ARG boxes.

In one embodiment, the feedback resistant argA gene is present on a plasmid and operatively linked to a promoter that is induced under low-oxygen or anaerobic conditions. In one embodiment, the feedback resistant argA gene is present in the chromosome and operatively linked to a promoter that is induced under low-oxygen or anaerobic conditions. In one embodiment, the feedback resistant argA gene is present on a plasmid and operatively linked to a promoter that is induced by exposure to tetracycline.

In one embodiment, the genetically engineered bacteria comprise an oxygen level-dependent transcriptional regulator, e.g., FNR, ANR, or DNR, and corresponding promoter from a different bacterial species. The non-native oxygen level-dependent transcriptional regulator and promoter increase the transcription of genes operatively linked to the said promoter, e.g., feedback resistant argA, in a low-oxygen or anaerobic environment, as compared to the native transcriptional regulator and promoter in the bacteria under the same conditions. In certain embodiments, the non-native oxygen level-dependent transcriptional regulator is an FNR protein from N. gonorrhoeae (see, e.g., Isabella et al., 2011). In one embodiment, the corresponding wild type transcriptional regulator is left intact and retains wild-type activity. In alternate embodiments, the corresponding wild-type transcriptional regulator is deleted or mutated to reduce or eliminate wild-type activity.

In one embodiment, the genetically engineered bacteria comprise a wild-type oxygen level-dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operatively linked to said promoter, e.g., feedback resistant argA, in a low-oxygen or anaerobic environment, as compared to the wild-type promoter under the same conditions. In one embodiment, the genetically engineered bacteria comprise a wild-type oxygen level-dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operatively linked to said promoter, e.g., feedback resistant argA, in a low-oxygen or anaerobic environment, as compared to the wild-type transcriptional regulator under the same conditions. In certain embodiments, the mutant oxygen level-dependent transcriptional regulator is an FNR protein comprising amino acid substitutions that enhance dimerization and FNR activity (see, e.g., Moore et al., 2006).

In certain embodiments, the genetically engineered bacteria comprise argA^{fbr} expressed under the control of an oxygen level-dependent promoter, e.g., the FNR promoter, and do not comprise wild-type argA. In one embodiment, the genetically engineered bacteria comprise argA^{fbr} expressed under the control of an oxygen level-dependent promoter, e.g., the FNR promoter, as well as wild-type argA expressed under the control of a wild-type argA regulatory region from unmodified bacteria of the same subtype.

In some embodiments, the genetically engineered bacteria express argA^{fbr} from a plasmid and/or chromosome. In some embodiments, the argA^{fbr} gene is expressed under the control of a constitutive promoter. In some embodiments, the argA^{fbr} gene is expressed under the control of an inducible promoter. In one embodiment, argA^{fbr} is expressed under the control of an oxygen level-dependent promoter that is activated under low-oxygen or anaerobic environments, e.g., a FNR promoter. The nucleic acid sequence of an exemplary FNR promoter-driven argA^{fbr} sequence is shown in Table 8. The FNR promoter sequence is bolded and the argA^{fbr} sequence is [symbol] . The nucleic acid sequence of a FNR promoter-driven argA^{fbr} plasmid is shown in Table 9, with the FNR promoter sequence bolded and argA^{fbr} sequence [symbol] . Table 10 shows the nucleic acid sequence of an exemplary pSC101 plasmid. Any suitable FNR promoter(s) may be combined with any suitable feedback-resistant ArgA. Non-limiting FNR promoter sequences are provided above. In some embodiments, the genetically engineered bacteria of the invention comprise one or more of: SEQ ID NO: 16, SEQ ID NO: 17, nirB1 promoter (SEQ ID NO: 18), nirB2 promoter (SEQ ID NO: 19), nirB3 promoter (SEQ ID NO: 20), ydfZ promoter (SEQ ID NO: 21), nirB promoter fused to a strong ribosome binding site (SEQ ID NO: 22), ydfZ promoter fused to a strong ribosome binding site (SEQ ID NO: 23), fnrS, an anaerobically induced small RNA gene (fnrS1 promoter SEQ ID NO: 24 or fnrS2 promoter SEQ ID NO: 25), nirB promoter fused to a crp binding site (SEQ ID NO: 26), and fnrS fused to a crp binding site (SEQ ID NO: 27). Table 11 depicts the nucleic acid sequence of an exemplary fnrS promoter-driven argA^{fbr} sequence. The FNR promoter sequence is bolded, the ribosome binding site is [symbol] and the argA sequence is [symbol] .

In some embodiments, the genetically engineered bacteria comprise the nucleic acid sequence of SEQ ID NO: 32 or a functional fragment thereof. In some embodiments, the genetically engineered bacteria comprise a nucleic acid sequence that, but for the redundancy of the genetic code, encodes the same polypeptide as SEQ ID NO: 32. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 32, or a nucleic acid sequence that, but for the redundancy of the genetic code, encodes the same polypeptide as SEQ ID NO: 32.

In some embodiments, the genetically engineered bacteria comprise the nucleic acid sequence of SEQ ID NO: 33 or a functional fragment thereof. In some embodiments, the genetically engineered bacteria comprise a nucleic acid sequence that, but for the redundancy of the genetic code, encodes the same polypeptide as SEQ ID NO: 33. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 33, or a nucleic acid sequence that, but for the redundancy of the genetic code, encodes the same polypeptide as SEQ ID NO: 33.

In some embodiments, the genetically engineered bacteria comprise the nucleic acid sequence of SEQ ID NO: 35 or a functional fragment thereof. In some embodiments, the genetically engineered bacteria comprise a nucleic acid sequence that, but for the redundancy of the genetic code, encodes the same polypeptide as SEQ ID NO: 35. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 35, or a nucleic acid sequence that, but for the redundancy of the genetic code, encodes the same polypeptide as SEQ ID NO: 35.

TABLE 8
Exemplary FNR promoter-driven argA^{fbr} sequence
(SEQ ID NO: 32)

AGTTGTTCTTATTGGTGGTGTTGCTTTATGGTTGCATCGTAGTAAATGGTTGTAACAAAAGCAATTTTT
CCGGCTGTCTGTATACAAAAACGCCGCAAAGTTTGAGCGAAGTCAATAAACTCTCTACCCATTCAGGGC
AATATCTCTCTTggatccaaagtgaactctagaaataattttgtttaactttaagaaggagatatacat

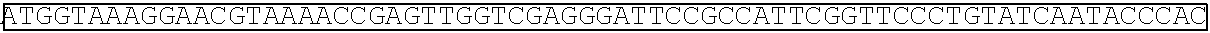

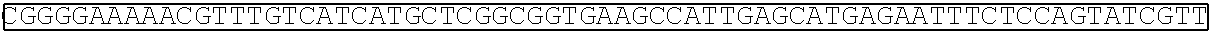

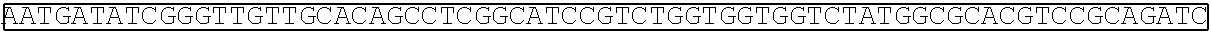

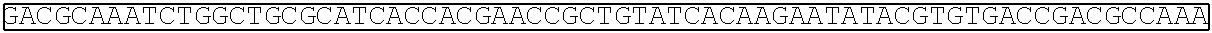

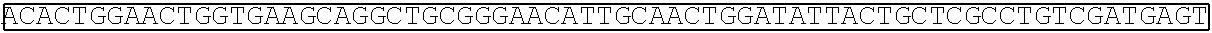

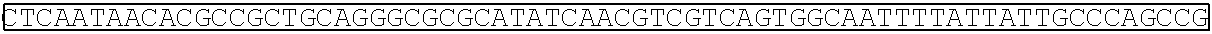

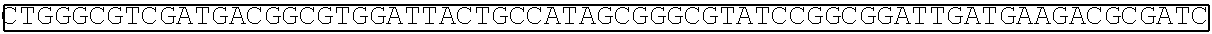

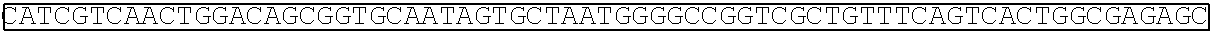

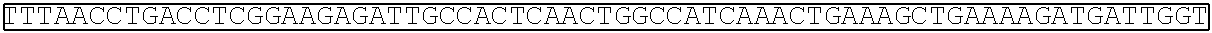

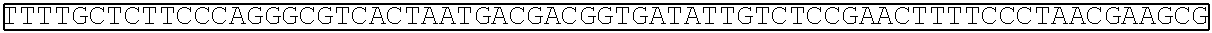

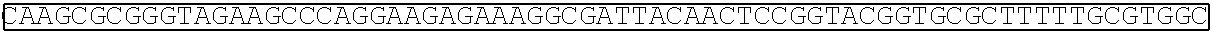

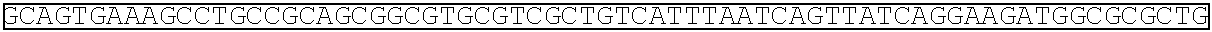

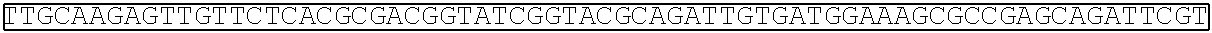

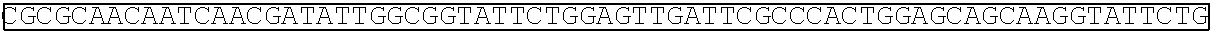

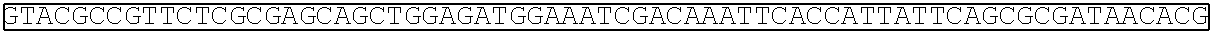

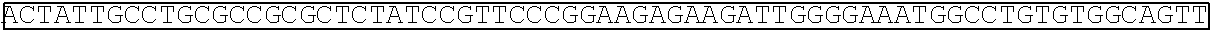

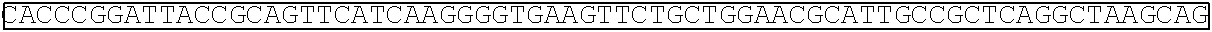

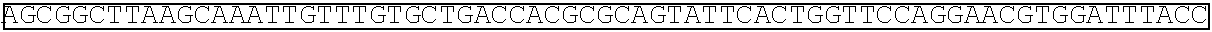

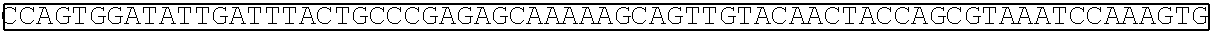

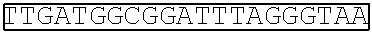

TABLE 9
Exemplary sequence of FNR promoter-driven argA^{fbr} plasmid
(SEQ ID NO: 33)

GTAAAACGACGGCCAGTGAATTCGAGCTCGGTACCATCCCCATCACTCTTGATGGAGATCAATTCCCCAAGCTGC
TAGAGCGTTACCTTGCCCTTAAACATTAGCAATGTCGATTTATCAGAGGGCCGACAGGCTCCCACAGGAGAAAAC

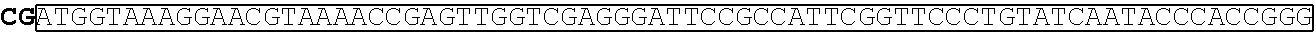

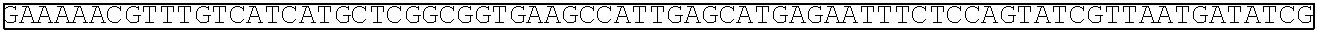

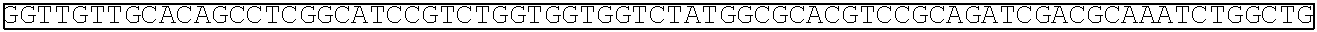

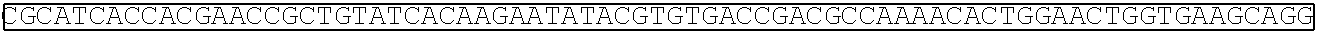

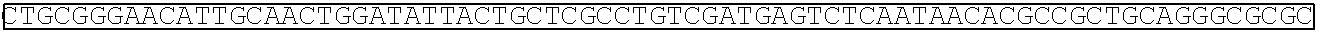

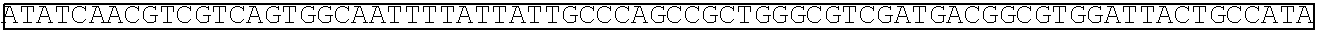

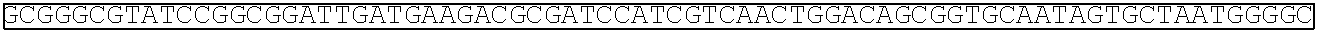

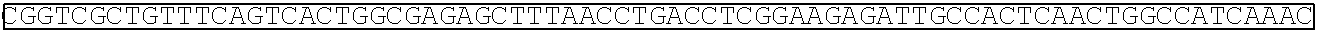

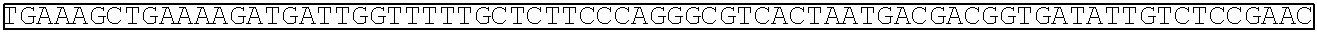

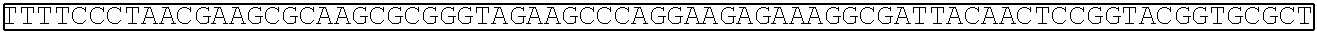

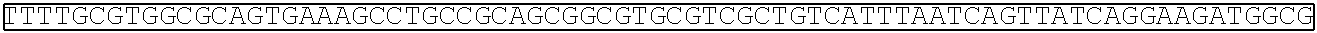

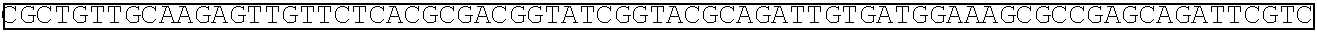

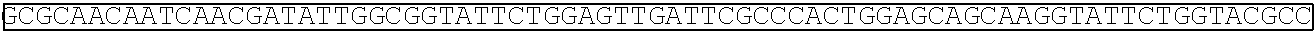

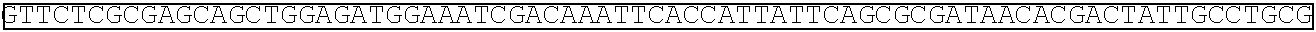

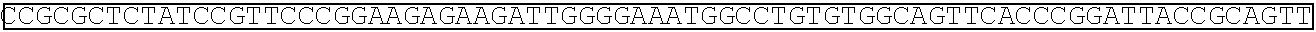

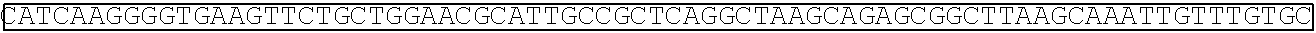

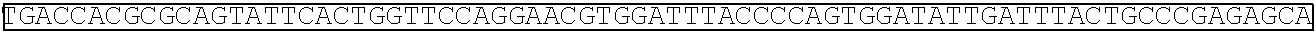

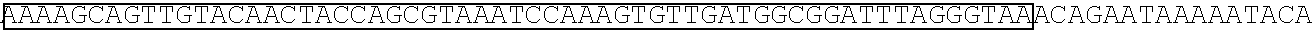

ATAATTTCGAATAATCATGCAAAGCTTGGCGTAATCATGGTCATAGCTGTTTCCTGTGTGAAATTGTTATCCGCT
CACAATTCCACACAACATACGAGCCGGAAGCATGTACGGGTTTTGCTGCCCGCAAACGGGCTGTTCTGGTGTTGC
TAGTTTGTTATCAGAATCGCAGATCCGGCTTCAGGTTTGCCGGCTGAAAGCGCTATTTCTTCCAGAATTGCCATG
ATTTTTTCCCCACGGGAGGCGTCACTGGCTCCCGTGTTGTCGGCAGCTTTGATTCGATAAGCAGCATCGCCTGTT
TCAGGCTGTCTATGTGTGACTGTTGAGCTGTAACAAGTTGTCTCAGGTGTTCAATTTCATGTTCTAGTTGCTTTG
TTTTACTGGTTTCACCTGTTCTATTAGGTGTTACATGCTGTTCATCTGTTACATTGTCGATCTGTTCATGGTGAA
CAGCTTTAAATGCACCAAAAACTCGTAAAAGCTCTGATGTATCTATCTTTTTTACACCGTTTTCATCTGTGCATA
TGGACAGTTTTCCCTTTGATATCTAACGGTGAACAGTTGTTCTACTTTTGTTTGTTAGTCTTGATGCTTCACTGA
TAGATACAAGAGCCATAAGAACCTCAGATCCTTCCGTATTTAGCCAGTATGTTCTCTAGTGTGGTTCGTTGTTTT
TGCGTGAGCCATGAGAACGAACCATTGAGATCATGCTTACTTTGCATGTCACTCAAAAATTTTGCCTCAAAACTG
GTGAGCTGAATTTTTGCAGTTAAAGCATCGTGTAGTGTTTTTCTTAGTCCGTTACGTAGGTAGGAATCTGATGTA
ATGGTTGTTGGTATTTTGTCACCATTCATTTTTATCTGGTTGTTCTCAAGTTCGGTTACGAGATCCATTTGTCTA
TCTAGTTCAACTTGGAAAATCAACGTATCAGTCGGGCGGCCTCGCTTATCAACCACCAATTTCATATTGCTGTAA
GTGTTTAAATCTTTACTTATTGGTTTCAAAACCCATTGGTTAAGCCTTTTAAACTCATGGTAGTTATTTTCAAGC
ATTAACATGAACTTAAATTCATCAAGGCTAATCTCTATATTTGCCTTGTGAGTTTTCTTTTGTGTTAGTTCTTTT
AATAACCACTCATAAATCCTCATAGAGTATTTGTTTTCAAAAGACTTAACATGTTCCAGATTATATTTTATGAAT
TTTTTTAACTGGAAAAGATAAGGCAATATCTCTTCACTAAAAACTAATTCTAATTTTTCGCTTGAGAACTTGGCA
TAGTTTGTCCACTGGAAAATCTCAAAGCCTTTAACCAAAGGATTCCTGATTTCCACAGTTCTCGTCATCAGCTCT
CTGGTTGCTTTAGCTAATACACCATAAGCATTTTCCCTACTGATGTTCATCATCTGAGCGTATTGGTTATAAGTG
AACGATACCGTCCGTTCTTTCCTTGTAGGGTTTTCAATCGTGGGGTTGAGTAGTGCCACACAGCATAAAATTAGC
TTGGTTTCATGCTCCGTTAAGTCATAGCGACTAATCGCTAGTTCATTTGCTTTGAAAACAACTAATTCAGACATA
CATCTCAATTGGTCTAGGTGATTTTAATCACTATACCAATTGAGATGGGCTAGTCAATGATAATTACTAGTCCTT
TTCCTTTGAGTTGTGGGTATCTGTAAATTCTGCTAGACCTTTGCTGGAAAACTTGTAAATTCTGCTAGACCCTCT
GTAAATTCCGCTAGACCTTTGTGTGTTTTTTTTGTTTATATTCAAGTGGTTATAATTTATAGAATAAAGAAAGAA
TAAAAAAAGATAAAAAGAATAGATCCCAGCCCTGTGTATAACTCACTACTTTAGTCAGTTCCGCAGTATTACAAA
AGGATGTCGCAAACGCTGTTTGCTCCTCTACAAAACAGACCTTAAAACCCTAAAGGCTTAAGTAGCACCCTCGCA
AGCTCGGGCAAATCGCTGAATATTCCTTTTGTCTCCGACCATCAGGCACCTGAGTCGCTGTCTTTTTCGTGACAT
TCAGTTCGCTGCGCTCACGGCTCTGGCAGTGAATGGGGGTAAATGGCACTACAGGCGCCTTTTATGGATTCATGC
AAGGAAACTACCCATAATACAAGAAAAGCCCGTCACGGGCTTCTCAGGGCGTTTTATGGCGGGTCTGCTATGTGG
TGCTATCTGACTTTTTGCTGTTCAGCAGTTCCTGCCCTCTGATTTTCCAGTCTGACCACTTCGGATTATCCCGTG
ACAGGTCATTCAGACTGGCTAATGCACCCAGTAAGGCAGCGGTATCATCAACAGGCTTACCCGTCTTACTGTCTT
TTCTACGGGGTCTGACGCTCAGTGGAACGAAAACTCACGTTAAGGGATTTTGGTCATGAGATTATCAAAAAGGAT
CTTCACCTAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTGCCTGACT
CCCCGTCGTGTAGATAACTACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCACTGCATAATTCTCTTACTGTCATGCC
ATCCGTAAGATGCTTTTCTGTGACTGGTGAGTACTCAACCAAGTCATTCTGAGAATAGTGTATGCGGCGACCGAG
TTGCTCTTGCCCGGCGTCAATACGGGATAATACCGCGCCACATAGCAGAACTTTAAAAGTGCTCATCATTGGAAA
ACGTTCTTCGGGGCGAAAACTCTCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAACAGGAAGGCAAAATGCCGCAAA
AAAGGGAATAAGGGCGACACGGAAATGTTGAATACTCATACTCTTCCTTTTTCAATATTATTGAAGCATTTATCA
GGGTTATTGTCTCATGAGCGGATACATATTTGAATGTATTTAGAAAAATAAACAAATAGGGGTTCCGCGCACATT
TCCCCGAAAAGTGCCACCTGACGTCTAAGAAACCATTATTATCATGACATTAACCTATAAAAATAGGCGTATCAC
GAGGCCCTTTCGTCTCGCGCGTTTCGGTGATGACGGTGAAAACCTCTGACACATGCAGCTCCCGGAGACGGTCAC
AGCTTGTCTGTAAGCGGATGCCGGGAGCAGACAAGCCCGTCAGGGCGCGTCAGCGGGTGTTGGCGGGTGTCGGGG
CTGGCTTAACTATGCGGCATCAGAGCAGATTGTACTGAGAGTGCACCATATGCGGTGTGAAATACCGCACAGATG
CGTAAGGAGAAAATACCGCATCAGGCGCCATTCGCCATTCAGGCTGCGCAACTGTTGGGAAGGGCGATCGGTGCG
GGCCTCTTCGCTATTACGCCAGCTGGCGAAAGGGGGATGTGCTGCAAGGCGATTAAGTTGGGTAACGCCAGGGTT
TTCCCAGTCACGACGTT

TABLE 10
Nucleic acid sequence of pSC101 plasmid (SEQ ID NO: 34)

ATTAAGTTGGGTAACGCCAGGGTTTTCCCAGTCACGACGTTATTGCGTTGCGCTCACTGCCCGCTTTCCAGTCGG
GAAACCTGTCGTGCCAGCTGCATTAATGAATCGGCCAACGCGCGGGGAGAGGCGGTTTGCGTATTGGGCGCTCTT
CCGCTTCCTCGCTCACTGACTCGCTGCGCTCGGTCGTTCGGCTGCGGCGAGCGGTATCAGCTCACTCAAAGGCGG
TAGTACGGGTTTTGCTGCCCGCAAACGGGCTGTTCTGGTGTTGCTAGTTTGTTATCAGAATCGCAGATCCGGCTT
CAGGTTTGCCGGCTGAAAGCGCTATTTCTTCCAGAATTGCCATGATTTTTTCCCCACGGGAGGCGTCACTGGCTC
CCGTGTTGTCGGCAGCTTTGATTCGATAAGCAGCATCGCCTGTTTCAGGCTGTCTATGTGTGACTGTTGAGCTGT
AACAAGTTGTCTCAGGTGTTCAATTTCATGTTCTAGTTGCTTTGTTTTACTGGTTTCACCTGTTCTATTAGGTGT
TACATGCTGTTCATCTGTTACATTGTCGATCTGTTCATGGTGAACAGCTTTAAATGCACCAAAAACTCGTAAAAG
CTCTGATGTATCTATCTTTTTTACACCGTTTTCATCTGTGCATATGGACAGTTTTCCCTTTGATATCTAACGGTG
AACAGTTGTTCTACTTTTGTTTGTTAGTCTTGATGCTTCACTGATAGATACAAGAGCCATAAGAACCTCAGATCC
TTCCGTATTTAGCCAGTATGTTCTCTAGTGTGGTTCGTTGTTTTTGCGTGAGCCATGAGAACGAACCATTGAGAT
CATGCTTACTTTGCATGTCACTCAAAAATTTTGCCTCAAAACTGGTGAGCTGAATTTTTGCAGTTAAAGCATCGT
GTAGTGTTTTTCTTAGTCCGTTACGTAGGTAGGAATCTGATGTAATGGTTGTTGGTATTTTGTCACCATTCATTT
TTATCTGGTTGTTCTCAAGTTCGGTTACGAGATCCATTTGTCTATCTAGTTCAACTTGGAAAATCAACGTATCAG
TCGGGCGGCCTCGCTTATCAACCACCAATTTCATATTGCTGTAAGTGTTTAAATCTTTACTTATTGGTTTCAAAA
CCCATTGGTTAAGCCTTTTAAACTCATGGTAGTTATTTTCAAGCATTAACATGAACTTAAATTCATCAAGGCTAA
TCTCTATATTTGCCTTGTGAGTTTTCTTTTGTGTTAGTTCTTTTAATAACCACTCATAAATCCTCATAGAGTATT
TGTTTTCAAAAGACTTAACATGTTCCAGATTATATTTTATGAATTTTTTTAACTGGAAAAGATAAGGCAATATCT
CTTCACTAAAAACTAATTCTAATTTTTCGCTTGAGAACTTGGCATAGTTTGTCCACTGGAAAATCTCAAAGCCTT
TAACCAAAGGATTCCTGATTTCCACAGTTCTCGTCATCAGCTCTCTGGTTGCTTTAGCTAATACACCATAAGCAT
TTTCCCTACTGATGTTCATCATCTGAGCGTATTGGTTATAAGTGAACGATACCGTCCGTTCTTTCCTTGTAGGGT
TTTCAATCGTGGGGTTGAGTAGTGCCACACAGCATAAAATTAGCTTGGTTTCATGCTCCGTTAAGTCATAGCGAC
TAATCGCTAGTTCATTTGCTTTGAAAACAACTAATTCAGACATACATCTCAATTGGTCTAGGTGATTTTAATCAC
TATACCAATTGAGATGGGCTAGTCAATGATAATTACTAGTCCTTTTCCTTTGAGTTGTGGGTATCTGTAAATTCT
GCTAGACCTTTGCTGGAAAACTTGTAAATTCTGCTAGACCCTCTGTAAATTCCGCTAGACCTTTGTGTGTTTTTT
TTGTTTATATTCAAGTGGTTATAATTTATAGAATAAAGAAAGAATAAAAAAAGATAAAAAGAATAGATCCCAGCC
CTGTGTATAACTCACTACTTTAGTCAGTTCCGCAGTATTACAAAAGGATGTCGCAAACGCTGTTTGCTCCTCTAC
AAAACAGACCTTAAAACCCTAAAGGCTTAAGTAGCACCCTCGCAAGCTCGGGCAAATCGCTGAATATTCCTTTTG
TCTCCGACCATCAGGCACCTGAGTCGCTGTCTTTTTCGTGACATTCAGTTCGCTGCGCTCACGGCTCTGGCAGTG
AATGGGGGTAAATGGCACTACAGGCGCCTTTTATGGATTCATGCAAGGAAACTACCCATAATACAAGAAAAGCCC
GTCACGGGCTTCTCAGGGCGTTTTATGGCGGGTCTGCTATGTGGTGCTATCTGACTTTTTGCTGTTCAGCAGTTC
CTGCCCTCTGATTTTCCAGTCTGACCACTTCGGATTATCCCGTGACAGGTCATTCAGACTGGCTAATGCACCCAG
TAAGGCAGCGGTATCATCAACAGGCTTACCCGTCTTACTGTCTTTTCTACGGGGTCTGACGCTCAGTGGAACGAA
AACTCACGTTAAGGGATTTTGGTCATGAGATTATCAAAAAGGATCTTCACCTAGATCCTTTTAAATTAAAAATGA
AGTTTTAAATCAATCTAAAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGATTTATCAGCAATA
AACCAGCCAGCCGGAAGGGCCGAGCGCAGAAGTGGTCCTGCAACTTTATCCGCCTCCATCCAGTCTATTAATTGT
TGCCGGGAAGCTAGAGTAAGTAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAAGGCGAGTTACATGATCCCCC
ATGTTGTGCAAAAAAGCGGTTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTCAATACGGGATAAT
ACCGCGCCACATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTTACTTTCACC
AGCGTTTCTGGGTGAGCAAAAACAGGAAGGCAAAATGCCGCAAAAAAGGGAATAAGGGCGACACGGAAATGTTGA
ATACTCATACTCTTCCTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACCTGACGTCTAAGAA
ACCATTATTATCATGACATTAACCTATAAAAATAGGCGTATCACGAGGCCCTTTCGTCTCGCGCGTTTCGGTGAT
GACGGTGAAAACCTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTATGCGGCATCAGAGCAGATT
GTACTGAGAGTGCACCATATGCGGTGTGAAATACCGCACAGATGCGTAAGGAGAAAATACCGCATCAGGCGCCAT
TCGCCATTCAGGCTGCGCAACTGTTGGGAAGGGCGATCGGTGCGGGCCTCTTCGCTATTACGCCAGCTGGCGAAA
GGGGGATGTGCTGCAAGGCG

TABLE 11
Nucleotide sequence of exemplary fnrS promoter-driven
argA^{fbr} pSC101 plasmid (SEQ ID NO: 35)

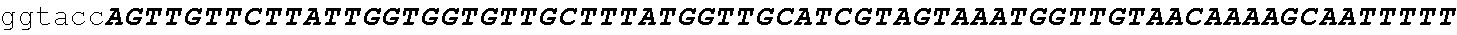

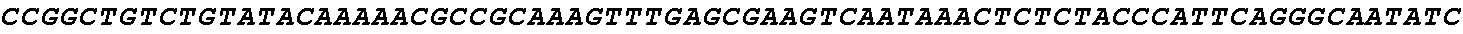

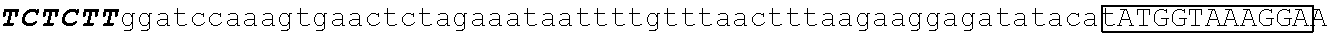

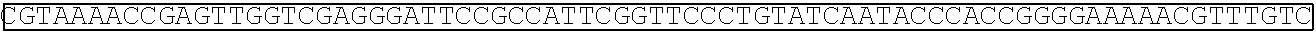

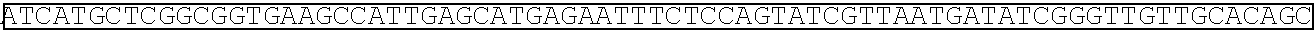

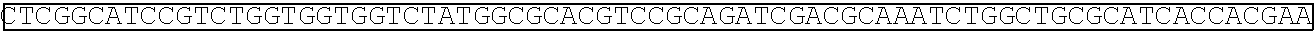

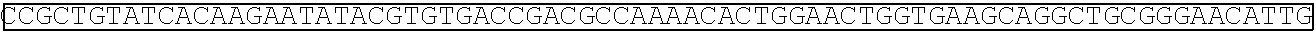

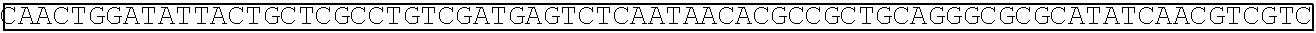

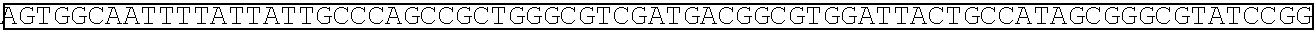

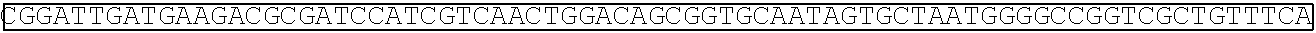

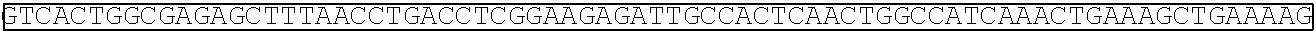

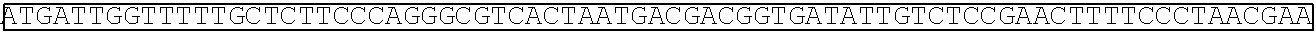

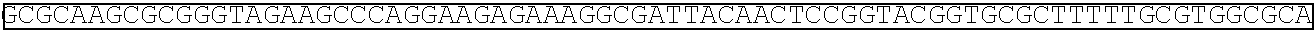

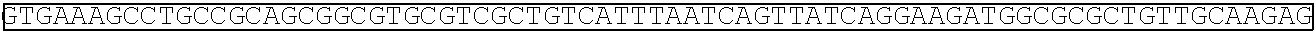

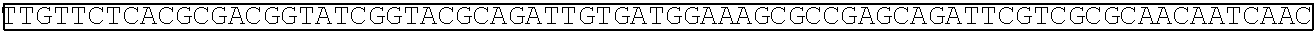

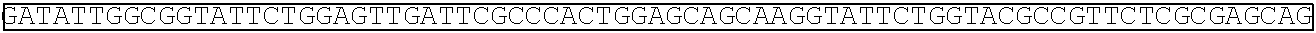

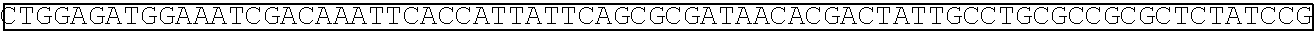

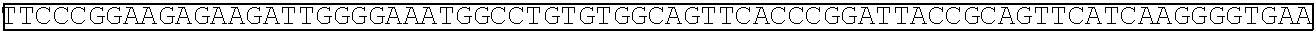

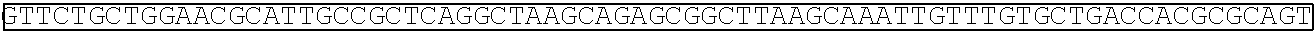

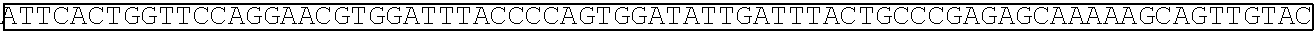

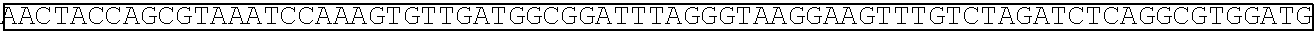

GCTTGGCGTAATCATGGTCATAGCTGTTTCCTGTGTGAAATTGTTATCCGCTCACAATTCCACACAACATACGAG
CCGGAAGCATAAAGTGTAAAGCCTGGGGTGCCTAATGAGTGAGCTAACTCACATTAATTGCGTTGCGCTCACTGC
CCGCTTTCCAGTCGGGAAACCTGTCGTGCCAGCTGCATTAATGAATCGGCCAACGCGCGGGGAGAGGCGGTTTGC
GTATTGGGCGCTCTTCCGCTTCCTCGCTCACTGACTCGCTGCGCTCGGTCGTTCGGCTGCGGCGAGCGGTATCAG
CTCACTCAAAGGCGGTAGTACGGGTTTTGCTGCCCGCAAACGGGCTGTTCTGGTGTTGCTAGTTTGTTATCAGAA
TCGCAGATCCGGCTTCAGGTTTGCCGGCTGAAAGCGCTATTTCTTCCAGAATTGCCATGATTTTTTCCCCACGGG
AGGCGTCACTGGCTCCCGTGTTGTCGGCAGCTTTGATTCGATAAGCAGCATCGCCTGTTTCAGGCTGTCTATGTG
TGACTGTTGAGCTGTAACAAGTTGTCTCAGGTGTTCAATTTCATGTTCTAGTTGCTTTGTTTTACTGGTTTCACC
TGTTCTATTAGGTGTTACATGCTGTTCATCTGTTACATTGTCGATCTGTTCATGGTGAACAGCTTTAAATGCACC
AAAAACTCGTAAAAGCTCTGATGTATCTATCTTTTTTACACCGTTTTCATCTGTGCATATGGACAGTTTTCCCTT
TGATATCTAACGGTGAACAGTTGTTCTACTTTTGTTTGTTAGTCTTGATGCTTCACTGATAGATACAAGAGCCAT
AAGAACCTCAGATCCTTCCGTATTTAGCCAGTATGTTCTCTAGTGTGGTTCGTTGTTTTTGCGTGAGCCATGAGA
ACGAACCATTGAGATCATGCTTACTTTGCATGTCACTCAAAAATTTTGCCTCAAAACTGGTGAGCTGAATTTTTG
CAGTTAAAGCATCGTGTAGTGTTTTTCTTAGTCCGTTACGTAGGTAGGAATCTGATGTAATGGTTGTTGGTATTT
TGTCACCATTCATTTTTATCTGGTTGTTCTCAAGTTCGGTTACGAGATCCATTTGTCTATCTAGTTCAACTTGGA
AAATCAACGTATCAGTCGGGCGGCCTCGCTTATCAACCACCAATTTCATATTGCTGTAAGTGTTTAAATCTTTAC
TTATTGGTTTCAAAACCCATTGGTTAAGCCTTTTAAACTCATGGTAGTTATTTTCAAGCATTAACATGAACTTAA
ATTCATCAAGGCTAATCTCTATATTTGCCTTGTGAGTTTTCTTTTGTGTTAGTTCTTTTAATAACCACTCATAAA
TCCTCATAGAGTATTTGTTTTCAAAAGACTTAACATGTTCCAGATTATATTTTATGAATTTTTTTAACTGGAAAA
GATAAGGCAATATCTCTTCACTAAAAACTAATTCTAATTTTTCGCTTGAGAACTTGGCATAGTTTGTCCACTGGA
AAATCTCAAAGCCTTTAACCAAAGGATTCCTGATTTCCACAGTTCTCGTCATCAGCTCTCTGGTTGCTTTAGCTA
ATACACCATAAGCATTTTCCCTACTGATGTTCATCATCTGAGCGTATTGGTTATAAGTGAACGATACCGTCCGTT
CTTTCCTTGTAGGGTTTTCAATCGTGGGGTTGAGTAGTGCCACACAGCATAAAATTAGCTTGGTTTCATGCTCCG
TTAAGTCATAGCGACTAATCGCTAGTTCATTTGCTTTGAAAACAACTAATTCAGACATACATCTCAATTGGTCTA
GGTGATTTTAATCACTATACCAATTGAGATGGGCTAGTCAATGATAATTACTAGTCCTTTTCCTTTGAGTTGTGG
GTATCTGTAAATTCTGCTAGACCTTTGCTGGAAAACTTGTAAATTCTGCTAGACCCTCTGTAAATTCCGCTAGAC
CTTTGTGTGTTTTTTTTGTTTATATTCAAGTGGTTATAATTTATAGAATAAAGAAAGAATAAAAAAAGATAAAAA
GAATAGATCCCAGCCCTGTGTATAACTCACTACTTTAGTCAGTTCCGCAGTATTACAAAAGGATGTCGCAAACGC
TGTTTGCTCCTCTACAAAACAGACCTTAAAACCCTAAAGGCTTAAGTAGCACCCTCGCAAGCTCGGGCAAATCGC
TGAATATTCCTTTTGTCTCCGACCATCAGGCACCTGAGTCGCTGTCTTTTTCGTGACATTCAGTTCGCTGCGCTC
ACGGCTCTGGCAGTGAATGGGGGTAAATGGCACTACAGGCGCCTTTTATGGATTCATGCAAGGAAACTACCCATA
ATACAAGAAAAGCCCGTCACGGGCTTCTCAGGGCGTTTTATGGCGGGTCTGCTATGTGGTGCTATCTGACTTTTT
GCTGTTCAGCAGTTCCTGCCCTCTGATTTTCCAGTCTGACCACTTCGGATTATCCCGTGACAGGTCATTCAGACT
GGCTAATGCACCCAGTAAGGCAGCGGTATCATCAACAGGCTTACCCGTCTTACTGTCTTTTCTACGGGGTCTGAC
GCTCAGTGGAACGAAAACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGACAGTTACCAATGCTTA
ATCAGTGAGGCACCTATCTCAGCGATCTGTCTATTTCGTTCATCCATAGTTGCCTGACTCCCCGTCGTGTAGATA
ACTACGATACGGGAGGGCTTACCATCTGGCCCCAGTGCTGCAATGATACCGCGAGACCCACGCTCACCGGCTCCA
GATTTATCAGCAATAAACCAGCCAGCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAATAGTTTGCGCAACGTTGTTGCCATT
GCTACAGGCATCGTGGTGTCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATCGTTGTCAGAAGTAAGTTG
GCCGCAGTGTTATCACTCATGGTTATGGCAGCACTGCATAATTCTCTTACTGTCATGCCATCCGTAAGATGCTTT
TCTGTGACTGGTGAGTACTCAACCAAGTCATTCTGAGAATAGTGTATGCGGCGACCGAGTTGCTCTTGCCCGGCG
TCAATACGGGATAATACCGCGCCACATAGCAGAACTTTAAAAGTGCTCATCATTGGAAAACGTTCTTCGGGGCGA
AAACTCTCAAGGATCTTACCGCTGTTGAGATCCAGTTCGATGTAACCCACTCGTGCACCCAACTGATCTTCAGCA
TCTTTTACTTTCACCAGCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGTTATTGTCTCATG
AGCGGATACATATTTGAATGTATTTAGAAAAATAAACAAATAGGGGTTCCGCGCACATTTCCCCGAAAAGTGCCA
CCTGACGTCTAAGAAACCATTATTATCATGACATTAACCTATAAAAATAGGCGTATCACGAGGCCCTTTCGTCTC
GCGCGTTTCGGTGATGACGGTGAAAACCTCTGACACATGCAGCTCCCGGAGACGGTCACAGCTTGTCTGTAAGCG
GATGCCGGGAGCAGACAAGCCCGTCAGGGCGCGTCAGCGGGTGTTGGCGGGTGTCGGGGCTGGCTTAACTATGCG
GCATCAGAGCAGATTGTACTGAGAGTGCACCATATGCGGTGTGAAATACCGCACAGATGCGTAAGGAGAAAATAC
CGCATCAGGCGCCATTCGCCATTCAGGCTGCGCAACTGTTGGGAAGGGCGATCGGTGCGGGCCTCTTCGCTATTA
CGCCAGCTGGCGAAAGGGGGATGTGCTGCAAGGCG

SEQ ID NO: 36 comprises FNRS-fbrArgA and chloramphenicol resistance. SEQ ID NO: 37 comprises FNRS-fbrArgA and kanamycin resistance. SEQ ID NO: 38 comprises FNRS-fbrArgA and no antibiotic resistance.

In some embodiments, the genetically engineered bacteria comprise the nucleic acid sequence of SEQ ID NO: 36 or a functional fragment thereof. In some embodiments, the genetically engineered bacteria comprise a nucleic acid sequence that, but for the redundancy of the genetic code, encodes the same polypeptide as SEQ ID NO: 36 or a functional fragment thereof. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 36 or a functional fragment thereof, or a nucleic acid sequence that, but for the redundancy of the genetic code, encodes the same polypeptide as SEQ ID NO: 37 or a functional fragment thereof.

In some embodiments, the genetically engineered bacteria comprise the nucleic acid sequence of SEQ ID NO: 37 or a functional fragment thereof. In some embodiments, the genetically engineered bacteria comprise a nucleic acid sequence that, but for the redundancy of the genetic code, encodes the same polypeptide as SEQ ID NO: 37 or a functional fragment thereof. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 37 or a functional fragment thereof, or a nucleic acid sequence that, but for the redundancy of the genetic code, encodes the same polypeptide as SEQ ID NO: 37 or a functional fragment thereof.

In some embodiments, the genetically engineered bacteria comprise the nucleic acid sequence of SEQ ID NO: 38 or a functional fragment thereof. In some embodiments, the genetically engineered bacteria comprise a nucleic acid sequence that, but for the redundancy of the genetic code, encodes the same polypeptide as SEQ ID NO: 38 or a functional fragment thereof. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 38 or a functional fragment thereof, or a nucleic acid sequence that, but for the redundancy of the genetic code, encodes the same polypeptide as SEQ ID NO: 38 or a functional fragment thereof.

The genetically modified bacteria lack any functional ArgR repressor. In one embodiment, the argR gene is deleted in the genetically modified bacteria. In one embodiment, the argR gene is mutated to inactivate ArgR repressor function. In one embodiment, under conditions where Arg^{f} is expressed, the genetically engineered bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more arginine and/or transcript of the gene(s) in the operon as compared to unmodified bacteria of the same subtype under the same conditions.

Strains comprising Feedback Resistant N-acetylglutamate Synthetase, inducible constructs thereof, and sequences are described in US Patent Publication US20160333326 and International Patent Publication WO2017139697, the contents of which is herein incorporated by reference in its entirety. Mutations and or deletions in ArgR are described in in US Patent Publication US20160333326 and International Patent Publication WO2017139697, the contents of which is herein incorporated by reference in its entirety. Such constructs mutations, and deletions may be used in strains of the current disclosure.

In one embodiment, the argA^{fbr} gene is expressed under the control of a constitutive promoter. In one embodiment, the argA^{fbr} gene is expressed under the control of an inducible promoter. In one embodiment, argA^{fbr} is expressed under the control of the FNR promoter, an oxygen level-dependent promoter that is activated under low-oxygen or anaerobic environments. The nucleic acid sequence of the FNR promoter-driven argA^{fbr} plasmid is shown in SEQ ID NO: 35. A nucleotide sequence of argA^{fbr} is shown in SEQ ID NO: 30.

In one embodiment, the genetically engineered bacteria comprise the nucleic acid sequence of SEQ ID NO: 30 or a functional fragment thereof. In one embodiment, the genetically engineered bacteria comprise a nucleic acid sequence that, but for the redundancy of the genetic code, encodes the same polypeptide as encoded by SEQ ID NO: 30. In one embodiment, genetically engineered bacteria comprise a nucleic acid sequence that is at least about 80%, at least about 85%, at least about 90%, at least about 95%, or at least about 99% homologous to the DNA sequence of SEQ ID NO: 30, or a nucleic acid sequence that, but for the redundancy of the genetic code, encodes the same polypeptide as encoded by SEQ ID NO: 30.

One of ordinary skill in the art would appreciate that the genetic modifications disclosed herein may be adapted for other species, strains, and subtypes of bacteria. It is known, for example, that “[a]rginine-mediated regulation is remarkably well conserved in very divergent bacteria, i.e., gram-negative bacteria, such as E. coli, Salmonella enterica serovar Typhimurium, Thermotoga, and Moritella profunda, and gram-positive bacteria, such as B. subtilis, Geobacillus stearothermophilus . . . and Streptomyces davuligerus” (Nicoloff et al., 2004). Furthermore, the arginine repressor is “universally conserved in bacterial genomes” (Makarova et al., 2001).

In one embodiment, quantitative PCR (qPCR) is used to amplify, detect, and/or quantify mRNA expression levels of the arginine biosynthesis genes. Primers specific for arginine biosynthesis genes, e.g., argA, argB, argC, argD, argE, argF, argG, argH, argI, argJ, carA, and carB, may be designed and used to detect mRNA in a sample according to methods known in the art (Fraga et al., 2008). In one embodiment, a fluorophore is added to a sample reaction mixture that may contain arg mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In one embodiment, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In one embodiment, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the arginine biosynthesis genes.

In any of these embodiments, a modified bacteria may produce at least about 0% to 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45%, 45% to 50%, 50% to 55%, 55% to 60%, 60% to 65%, 65% to 70% to 80%, 80% to 90%, or 90% to 100% more L-arginine, than unmodified bacteria of the same bacterial subtype under the same conditions.

In yet another embodiment, a modified bacteria may produce at least about 1.0-1.2-fold, 1.2-1.4-fold, 1.4-1.6-fold, 1.6-1.8-fold, 1.8-2-fold, or two-fold more L-arginine, than unmodified bacteria of the same bacterial subtype under the same conditions. In yet another embodiment, a modified bacteria may produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more L-arginine, than unmodified bacteria of the same bacterial subtype under the same conditions.

In any of these embodiments, a modified bacteria may consume 0% to 2%, 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45% 45% to 50%, 50% to 55%, 55% to 60%, 60% to 65%, 65% to 70% to 80%, 80% to 90%, or 90% to 100% more ammonia than unmodified bacteria of the same bacterial subtype under the same conditions. In yet another embodiment, a modified bacteria may consume 1.0-1.2-fold, 1.2-1.4-fold, 1.4-1.6-fold, 1.6-1.8-fold, 1.8-2-fold, or two-fold more ammonia than unmodified bacteria of the same bacterial subtype under the same conditions. In yet another embodiment, a modified bacteria may consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more ammonia than unmodified bacteria of the same bacterial subtype under the same conditions.

In one embodiment, the bacterium is a modified bacterium, as described supra. In one embodiment, the modified bacterium is capable of producing specific levels of arginine. In one embodiment, the bacterium is a wild-type bacterium capable of producing specific levels of arginine.

In one embodiment, the bacterium produces or is capable of producing at least about 200 μM arginine in vitro after 3 hours. In one embodiment, the bacterium produces or is capable of producing at least about 250 μM arginine in vitro after 3 hours. In one embodiment, the bacterium produces or is capable of producing at least about 300 μM arginine in vitro after 3 hours. In one embodiment, the bacterium produces or is capable of producing at least about 350 μM arginine in vitro after 3 hours. In one embodiment, the bacterium produces or is capable of producing at least about 400 μM arginine in vitro after 3 hours. In one embodiment, the bacterium produces or is capable of producing at least about 450 μM arginine in vitro after 3 hours. In one embodiment, the bacterium produces or is capable of producing at least about 500 μM arginine in vitro after 3 hours. In one embodiment, the bacterium produces or is capable of producing at least about 550 μM arginine in vitro after 3 hours. In one embodiment, the bacterium produces or is capable of producing at least about 600 μM arginine in vitro after 3 hours.

In one embodiment, the bacterium produces or is capable of producing at least about 10 μM arginine in vitro after 1.5 hours. In one embodiment, the bacterium produces or is capable of producing at least about 25 μM arginine in vitro after 1.5 hours. In one embodiment, the bacterium produces or is capable of producing at least about 50 μM arginine in vitro after 1.5 hours. In one embodiment, the bacterium produces or is capable of producing at least about 100 μM arginine in vitro after 1.5 hours. In one embodiment, the bacterium produces or is capable of producing at least about 150 μM arginine in vitro after 1.5 hours. In one embodiment, the bacterium produces or is capable of producing at least about 200 μM arginine in vitro after 1.5 hours. In one embodiment, the bacterium produces or is capable of producing at least about 250 μM arginine in vitro after 1.5 hours. In one embodiment, the bacterium produces or is capable of producing at least about 300 μM arginine in vitro after 1.5 hours.

In one embodiment, the bacterium produces or is capable of producing between at least about 10 μM to at least about 600 μM arginine in vitro between 1.5 to 3 hours. In one embodiment, the bacterium produces or is capable of producing between at least about 25 μM to at least about 600 μM arginine in vitro between 1.5 to 3 hours. In one embodiment, the bacterium produces or is capable of producing between at least about 50 μM to at least about 600 μM arginine in vitro between 1.5 to 3 hours. In one embodiment, the bacterium produces or is capable of producing between at least about 100 μM to at least about 600 μM arginine in vitro between 1.5 to 3 hours. In one embodiment, the bacterium produces or is capable of producing between at least about 150 μM to at least about 600 μM arginine in vitro between 1.5 to 3 hours. In one embodiment, the bacterium produces or is capable of producing between at least about 200 μM to at least about 600 μM arginine in vitro between 1.5 to 3 hours. In one embodiment, the bacterium produces or is capable of producing between at least about 250 μM to at least about 600 μM arginine in vitro between 1.5 to 3 hours. In one embodiment, the bacterium produces or is capable of producing between at least about 300 μM to at least about 600 μM arginine in vitro between 1.5 to 3 hours. In one embodiment, the bacterium produces or is capable of producing between at least about 350 μM to at least about 600 μM arginine in vitro between 1.5 to 3 hours. In one embodiment, the bacterium produces or is capable of producing between at least about 400 μM to at least about 600 μM arginine in vitro between 1.5 to 3 hours. In one embodiment, the bacterium produces or is capable of producing between at least about 450 μM to at least about 600 μM arginine in vitro between 1.5 to 3 hours. In one embodiment, the bacterium produces or is capable of producing between at least about 500 μM to at least about 600 μM arginine in vitro between 1.5 to 3 hours. In one embodiment, the bacterium produces or is capable of producing between at least about 550 μM to at least about 600 μM arginine in vitro between 1.5 to 3 hours.

In one embodiment, the bacterium produces or is capable of producing at least about 25 μg of arginine per gram of tumor. In one embodiment, the bacterium produces or is capable of producing at least about 20 μg of arginine per gram of tumor. In one embodiment, the bacterium produces or is capable of producing at least about 15 μg of arginine per gram of tumor. In one embodiment, the bacterium produces or is capable of producing at least about 10 μg of arginine per gram of tumor. In one embodiment, the bacterium produces or is capable of producing at least about 5 μg of arginine per gram of tumor. In one embodiment, the bacterium produces or is capable of producing at least about 3 μg of arginine per gram of tumor.

In one embodiment, the bacterium produces or is capable of producing between at least about 3 μg of arginine per gram of tumor to at least about 25 μg of arginine per gram of tumor. In one embodiment, the bacterium produces or is capable of producing between at least about 5 μg of arginine per gram of tumor to at least about 25 μg of arginine per gram of tumor. In one embodiment, the bacterium produces or is capable of producing between at least about 10 μg of arginine per gram of tumor to at least about 25 μg of arginine per gram of tumor. In one embodiment, the bacterium produces or is capable of producing between at least about 15 μg of arginine per gram of tumor to at least about 25 μg of arginine per gram of tumor. In one embodiment, the bacterium produces or is capable of producing between at least about 20 μg of arginine per gram of tumor to at least about 25 μg of arginine per gram of tumor.

Other arginine-producing bacteria and/or ammonia consuming bacteria are known in the art (see, for example, Park et al., Nature Communications, 5: Article number 4618 (2014); U.S. Pat. No. 3,849,250; and Utagawa, Journal of Nutrition, 134(10): 2854S-2857S (2004); the entire contents of each of which are expressly incorporated herein by reference in their entireties).

Auxotrophs

An auxotrophic modification is intended to cause bacteria to die in the absence of an exogenously added nutrient essential for survival or growth because they lack the gene(s) necessary to produce that essential nutrient. In one embodiment, any of the bacteria described herein also comprise a deletion or mutation in a gene required for cell survival and/or growth.

In one embodiment, the essential gene is a DNA synthesis gene, for example, thyA. In another embodiment, the essential gene is a bacterial cell wall synthesis gene, for example, dapA. In yet another embodiment, the essential gene is an amino acid gene, for example, serA or metA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thil, as long as the corresponding wild-type gene product is not produced in the bacteria. Exemplary bacterial genes which may be disrupted or deleted to produce an auxotrophic strain as described in International Patent Application PCT/US2017/013072, filed Jan. 11, 2017, published as WO2017/123675, the contents of which is herein incorporated by reference in its entirety. These include, but are not limited to, genes required for oligonucleotide synthesis, amino acid synthesis, and cell wall synthesis. Table 12 lists exemplary bacterial genes which may be disrupted or deleted to produce an auxotrophic strain. These include, but are not limited to, genes required for oligonucleotide synthesis, amino acid synthesis, and cell wall synthesis.

TABLE 12
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Auxotrophic mutations are useful in some instances in which biocontainment strategies may be required to prevent unintended proliferation of the genetically engineered bacterium in a natural ecosystem. Any auxotrophic mutation in an essential gene described above or known in the art can be useful for this purpose, e.g. DNA synthesis genes, amino acid synthesis genes, or genes for the synthesis of cell wall. Accordingly, In one embodiment, the genetically engineered bacteria comprise modifications, e.g., mutation(s) or deletion(s) in one or more auxotrophic genes, e.g., to prevent growth and proliferation of the bacterium in the natural environment. In one embodiment, the modification may be located in a non-coding region. In one embodiment, the modifications result in attenuation of transcription or translation. In one embodiment, the modifications, e.g., mutations or deletions, result in reduced or no transcription or reduced or no translation of the essential gene. In one embodiment, the modifications, e.g., mutations or deletions, result in transcription and/or translation of a non-functional version of the essential gene. In one embodiment, the modifications, e.g., mutations or deletions result in in truncated transcription or translation of the essential gene, resulting in a truncated polypeptide. In one embodiment, the modification, e.g., mutation is located within the coding region of the gene.

While unable to grow in the natural ecosystem, certain auxotrophic mutations may allow growth and proliferation in the mammalian host administered the bacteria, e.g., in the tumor environment. For example, an essential pathway that is rendered non-functional by the auxotrophic mutation may be complemented by production of the metabolite by the host within the tumor microenvironment. As a result, the bacterium administered to the host can take up the metabolite from the environment and can proliferate and colonize the tumor. Thus, In one embodiment, the auxotrophic gene is an essential gene for the production of a metabolite, which is also produced by the mammalian host in vivo, e.g., in a tumor setting. In one embodiment, metabolite production by the host tumor may allow uptake of the metabolite by the bacterium and permit survival and/or proliferation of the bacterium within the tumor. In one embodiment, bacteria comprising such auxotrophic mutations are capable of proliferating and colonizing the tumor to the same extent as a bacterium of the same subtype which does not carry the auxotrophic mutation.

In one embodiment, the bacteria are capable of colonizing and proliferating in the tumor microenvironment. In one embodiment, the tumor colonizing bacteria comprise one or more auxotrophic mutations. In one embodiment, the tumor colonizing bacteria do not comprise one or more auxotrophic modifications or mutations. In a non-limiting example, greater numbers of bacteria are detected after 24 hours and 72 hours than were originally injected into the subject. In one embodiment, CFUs detected 24 hours post injection are at least about 1 to 2 logs greater than administered. In one embodiment, CFUs detected 24 hours post injection are at least about 2 to 3 logs greater than administered. In one embodiment, CFUs detected 24 hours post injection are at least about 3 to 4 logs greater than administered. In one embodiment, CFUs detected 24 hours post injection are at least about 4 to 5 logs greater than administered. In one embodiment, CFUs detected 24 hours post injection are at least about 5 to 6 logs greater than administered. In one embodiment, CFUs detected 72 hours post injection are at least about 1 to 2 logs greater than administered. In one embodiment, CFUs detected 72 hours post injection are at least about 2 to 3 logs greater than administered. In one embodiment, CFUs detected 72 hours post injection are at least about 3 to 4 logs greater than administered. In one embodiment, CFUs detected 72 hours post injection are at least about 4 to 5 logs greater than administered. In one embodiment, CFUs detected 72 hours post injection are at least about 5 to 6 logs greater than administered. In one embodiment, CFUs can be measured at later time points, such as after at least one week, after at least 2 or more weeks, after at least one month, after at least two or more months post injection.

Non-limiting examples of such auxotrophic genes, which allow proliferation and colonization of the tumor, are thyA and uraA, as shown herein. Accordingly, In one embodiment, the genetically engineered bacteria of the disclosure may comprise an auxotrophic modification, e.g., mutation or deletion, in the thyA gene. In one embodiment, the genetically engineered bacteria of the disclosure may comprise an auxotrophic modification, e.g., mutation or deletion, in the uraA gene. In one embodiment, the genetically engineered bacteria of the disclosure may comprise auxotrophic modification, e.g., mutation or deletion, in the thyA gene and the uraA gene.

Alternatively, the auxotrophic gene is an essential gene for the production of a metabolite which cannot be produced by the host within the tumor, i.e., the auxotrophic mutation is not complemented by production of the metabolite by the host within the tumor microenvironment. As a result, the this mutation may affect the ability of the bacteria to grow and colonize the tumor and bacterial counts decrease over time. This type of auxotrophic mutation can be useful for the modulation of in vivo activity of the checkpoint inhibitor or duration of activity of the checkpoint inhibitor, e.g., within a tumor. An example of this method of fine-tuning levels and timing of checkpoint inhibitor release is described herein using a auxotrophic modification, e.g., mutation, in dapA. Diaminopimelic acid (Dap) is a characteristic component of certain bacterial cell walls, e.g., of gram negative bacteria. Without diaminopimelic acid, bacteria are unable to form proteoglycan, and as such are unable to grow. DapA is not produced by mammalian cells, and therefore no alternate source of DapA is provided in the tumor. As such, a dapA auxotrophy may present a particularly useful strategy to modulate and fine tune timing and extent of bacterial presence in the tumor. Accordingly, In one embodiment, the genetically engineered bacteria of the disclosure comprise an mutation in an essential gene for the production of a metabolite which cannot be produced by the host within the tumor. In one embodiment, the auxotrophic mutation is in a gene which is essential for the production and maintenance of the bacterial cell wall known in the art or described herein, or a mutation in a gene that is essential to another structure that is unique to bacteria and not present in mammalian cells. In one embodiment, bacteria comprising such auxotrophic mutations are capable of proliferating and colonizing the tumor to a substantially lesser extent than a bacterium of the same subtype which does not carry the auxotrophic mutation. Control of bacterial growth (and by extent effector levels) may be further combined with other regulatory strategies, including but not limited to, metabolite or chemically inducible promoters described herein.

In a non-limiting example, lower numbers of bacteria are detected after 24 hours and 72 hours than were originally injected into the subject. In one embodiment, CFUs detected 24 hours post injection are at least about 1 to 2 logs lower than administered. In one embodiment, CFUs detected 24 hours post injection are at least about 2 to 3 logs lower than administered. In one embodiment, CFUs detected 24 hours post injection are at least about 3 to 4 logs lower than administered. In one embodiment, CFUs detected 24 hours post injection are at least about 4 to 5 logs lower than administered. In one embodiment, CFUs detected 24 hours post injection are at least about 5 to 6 logs lower than administered. In one embodiment, CFUs detected 72 hours post injection are at least about 1 to 2 logs lower than administered. In one embodiment, CFUs detected 72 hours post injection are at least about 2 to 3 logs lower than administered. In one embodiment, CFUs detected 72 hours post injection are at least about 3 to 4 logs lower than administered. In one embodiment, CFUs detected 72 hours post injection are at least about 4 to 5 logs lower than administered. In one embodiment, CFUs detected 72 hours post injection are at least about 5 to 6 logs lower than administered. In one embodiment, CFUs can be measured at later time points, such as after at least one week, after at least 2 or more weeks, after at least one month, after at least two or more months post injection.

In one embodiment, the bacteria of the disclosure comprise a auxotrophic modification, e.g., mutation, in dapA. trpE is another auxotrophic mutation described herein. Bacteria carrying this mutation cannot produce tryptophan. In one embodiment, the bacteria comprise auxotrophic mutation(s) in one essential gene. In one embodiment, the bacteria comprise auxotrophic mutation(s) in two essential genes (double auxotrophy). In one embodiment, the bacteria comprise auxotrophic mutation(s) in three or more essential gene(s).

Auxotrophic modifications may also be used to screen for mutant bacteria that produce the effector molecule for various applications. In one example, the auxotrophy is useful to monitor purity or “sterility” of batches in small and large scale production of a bacterial strain. In this case, the auxotrophy presents a means to distinguish the engineered bacterium from a potential contaminant. In a non-limiting example, during the manufacturing process of the live biotherapeutic (i.e., large scale), an auxotrophy can be a useful tool to demonstrate purity or “sterility” of the drug substance. This method to determine purity of the culture is particularly useful in the absence of an antibiotic resistance gene, which is often used for this purpose in experimental strains, but which may be removed during the development of the live therapeutic drug product.

trpE is another auxotrophic mutation described herein. Bacteria carrying this mutation cannot produce tryptophan. Genetically engineered bacteria described herein with a trpE mutation further comprise kynureninase. Kynureninase allows the bacterium to convert kynurenine into the tryptophan precursor anthranilate and therefore the bacterium can grow in the absence of tryptophan if kynurenine is present.

In one embodiment, the genetically engineered bacteria comprise auxotrophic mutation(s) in dapA and thyA. In one embodiment, the genetically engineered bacteria comprise auxotrophic mutation(s) in dapA and uraA. In one embodiment, the genetically engineered bacteria comprise auxotrophic mutation(s) in thyA and uraA. In one embodiment, the genetically engineered bacteria comprise auxotrophic mutation(s) in dapA, thyA and uraA.

In one embodiment, the genetically engineered bacteria comprise auxotrophic mutation(s) in trpE. In one embodiment, the genetically engineered bacteria comprise auxotrophic mutation(s) in trpE and thyA. In one embodiment, the genetically engineered bacteria comprise auxotrophic mutation(s) in trpE and dapA. In one embodiment, the genetically engineered bacteria comprise auxotrophic mutation(s) in trpE and uraA. In one embodiment, the genetically engineered bacteria comprise auxotrophic mutation(s) in trpE, dapA and thyA. In one embodiment, the genetically engineered bacteria comprise auxotrophic mutation(s) in trpE, dapA and uraA. In one embodiment, the genetically engineered bacteria comprise auxotrophic mutation(s) in trpE, thyA and uraA. In one embodiment, the genetically engineered bacteria comprise auxotrophic mutation(s) in trpE, dapA, thyA and uraA.

In another non-limiting example, a conditional auxotroph can be generated. The chromosomal copy of dapA or thyA is knocked out. Another copy of thyA or dapA is introduced, e.g., under control of a low oxygen promoter. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can also be employed to allow survival of bacteria under anaerobic conditions, e.g., the gut or conditions of the tumor microenvironment, but prevent survival under aerobic conditions.

In one embodiment, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain,” ACS Synthetic Biology (2015) DOI: 10.1021/acssynbio.5b00085, the entire contents of which are expressly incorporated herein by reference). SLiDE bacterial cells are described in International Patent Application PCT/US2017/013072, filed Jan. 11, 2017, published as WO2017/123675, the contents of which is herein incorporated by reference in its entirety.

In one embodiment, the genetically engineered bacteria also comprise a kill switch. Suitable kill switches are described in International Patent Application PCT/US2016/39427, filed Jun. 24, 2016, published as WO2016/210373, the contents of which are herein incorporated by reference in their entirety. The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

In one embodiment, the genetically engineered bacteria also comprise a plasmid that has been modified to create a host-plasmid mutual dependency. In certain embodiments, the mutually dependent host-plasmid platform is as described in Wright et al., 2015. These and other systems and platforms are described in International Patent Application PCT/US2017/013072, filed Jan. 11, 2017, published as WO2017/123675, the contents of which is herein incorporated by reference in its entirety.

### Checkpoint Inhibition

The arginine-producing bacteria and/or ammonia-consuming bacteria disclosed herein are provided in combination with one or more checkpoint inhibitors to produce a surprising and striking synergistic effect on tumors. In certain embodiments, the one or more arginine-producing bacteria and/or ammonia-consuming bacteria are administered sequentially, simultaneously, or subsequently to dosing with one or more checkpoint inhibitors.

As used herein, a “checkpoint inhibitor” refers to one or more therapeutic substances or drugs that are capable of reducing and/or inhibiting cell growth or replication. In particular, checkpoint inhibitors are useful for inhibiting the development of immunity to cancer. The generation of immunity to cancer is a potentially self-propagating cyclic process which has been referred to as the “Cancer-Immunity Cycle” (Chen and Mellman, Oncology Meets Immunology: The Cancer-Immunity Cycle; Immunity (2013) 39, 1-10), and which can lead to the broadening and amplification of the T cell response. The cycle is counteracted by inhibitory factors that lead to immune regulatory feedback mechanisms at various steps of the cycle and which can halt the development or limit the immunity.

The cycle essentially comprises a series of steps which need to occur for an anticancer immune response to be successfully mounted. The cycle includes steps, which must occur for the immune response to be initiated and a second series of events which must occur subsequently, in order for the immune response to be sustained (i.e., allowed to progress and expand and not dampened). These steps have been referred to as the “Cancer-Immunity Cycle” (Chen and Mellman, 2013), and are essentially as follows:

1. Release (oncolysis) and/or acquisition of tumor cell contents; Tumor cells break open and spill their contents, resulting in the release of neoantigens, which are taken up by antigen presenting cells (dendritic cells and macrophages for processing. Alternatively, antigen presenting cells may actively phagocytose tumors cells directly.

2. Activation of antigen presenting cells (APC) (dendritic cells and macrophages); In addition to the first step described above, the next step must involve release of proinflammatory cytokines or generation of proinflammatory cytokines as a result of release of DAMPs or PAMPs from the dying tumor cells to result in antigen presenting cell activation and subsequently an anticancer T cell response. Antigen presenting cell activation is critical to avoid peripheral tolerance to tumor derived antigens. If properly activated, antigen presenting cells present the previously internalized antigens on their surface in the context of MHCI and MHCII molecules alongside the proper co-stimulatory signals (CD80/86, cytokines, etc.) to prime and activate T cells.

3. Priming and Activation of T cells: Antigen presentation by DCs and macrophages causes the priming and activation of effector T cell responses against the cancer-specific antigens, which are seen as “foreign” by the immune system. This step is critical to the strength and breadth of the anti-cancer immune response, by determining quantity and quality of T effector cells and contribution of T regulatory cells. Additionally, proper priming of T cells can result in superior memory T cell formation and long lived immunity.

4. Trafficking and Infiltration: Next, the activated effector T cells must traffic to the tumor and infiltrate the tumor.

5. Recognition of cancer cells by T cells and T cell support, and augmentation and expansion of effector T cell responses: Once arrived at the tumor site, the T cells can recognize and bind to cancer cells via their T cell receptors (TCR), which specifically bind to their cognate antigen presented within the context of MHC molecules on the cancer cells, and subsequently kill the target cancer cell. Killing of the cancer cell releases tumor associated antigens through lysis of tumor cells, and the cycle re-initiates, thereby increasing the volume of the response in subsequent rounds of the cycle. Antigen recognition by either MHC-I or MHC-II restricted T cells can result in additional effector functions, such as the release of chemokines and effector cytokines, further potentiating a robust antitumor response.

6. Overcoming immune suppression: Finally, overcoming certain deficiencies in the immune response to the cancer and/or overcoming the defense strategy of the cancer, i.e., overcoming the breaks that the cancer employs in fighting the immune response, can be viewed as another critical step in the cycle. In some cases, even though T cell priming and activation has occurred, other immunosuppressive cell subsets are actively recruited and activated to the tumor microenvironment, i.e., regulatory T cells or myeloid derived suppressor cells. In other cases, T cells may not receive the right signals to properly home to tumors or may be actively excluded from infiltrating the tumor. Finally, certain mechanisms in the tumor microenvironment exist, which are capable of suppressing or repressing the effector cells that are produced as a result of the cycle. Such resistance mechanisms co-opt immune-inhibitory pathways, often referred to as immune checkpoints, which normally mediate immune tolerance and mitigate cancer tissue damage (see e.g., Pardoll (2012), The blockade of immune checkpoints in cancer immunotherapy; Nature Reviews Cancer volume 12, pages 252-264).

Therapies have been developed to promote and support progression through the cancer-immunity cycle at one or more of the 6 steps. These therapies can be broadly classified as therapies that promote initiation of the immune response and therapies that help sustain the immune response.

Non-limiting examples of immune checkpoint inhibitors useful herein include CTLA-4 antibodies (including but not limited to Ipilimumab and Tremelimumab (CP675206)), anti-4-1BB (CD137, TNFRSF9) antibodies (including but not limited to PF-05082566, and Urelumab), anti CD134 (OX40) antibodies, including but not limited to Anti-OX40 antibody (Providence Health and Services), anti-PD-1 antibodies (including but not limited to Nivolumab, Pidilizumab, Pembrolizumab (MK-3475/SCH900475, lambrolizumab, REGN2810, PD-1 (Agenus)), anti-PD-L1 antibodies (including but not limited to durvalumab (MEDI4736), avelumab (MSB0010718C), and atezolizumab (MPDL3280A, RG7446, RO5541267)), and anti-KIR antibodies (including but not limited to Lirilumab), LAG3 antibodies (including but not limited to BMS-986016), anti-CCR4 antibodies (including but not limited to Mogamulizumab), anti-CD27 antibodies (including but not limited to Varlilumab), anti-CXCR4 antibodies (including but not limited to Ulocuplumab). In one embodiment, the at least one bacterial cell is administered sequentially, simultaneously, or subsequently to dosing with an anti-phosphatidyl serine antibody (including but not limited to Bavituxumab).

In one embodiment, the at least one bacterial cell is administered sequentially, simultaneously, or subsequently to dosing with one or more antibodies selected from TLR9 antibody (including, but not limited to, MGN1703 PD-1 antibody (including, but not limited to, SHR-1210 (Incyte/Jiangsu Hengrui)), anti-OX40 antibody (including, but not limited to, OX40 (Agenus)), anti-Tim3 antibody (including, but not limited to, Anti-Tim3 (Agenus/INcyte)), anti-Lag3 antibody (including, but not limited to, Anti-Lag3 (Agenus/INcyte)), anti-B7H3 antibody (including, but not limited to, Enoblituzumab (MGA-271), anti-CT-011 (hBAT, hBAT1) as described in WO2009101611, anti-PDL-2 antibody (including, but not limited to, AMP-224 (described in WO2010027827 and WO2011066342)), anti-CD40 antibody (including, but not limited to, CP-870, 893), anti-CD40 antibody (including, but not limited to, CP-870, 893).

In one embodiment, the one or more arginine-producing bacterium and/or ammonia-consuming bacterium are administered sequentially, simultaneously, or subsequently to dosing with one or more checkpoint inhibitors. In one embodiment, the checkpoint inhibitor is administered systemically, and the bacteria are administered intratumorally. In one embodiment, the checkpoint inhibitor and bacteria are administered systemically. In one embodiment, one or more engineered bacteria described herein are administered sequentially, simultaneously, or subsequently to dosing with a checkpoint inhibitor, e.g., an anti-PD1 antibody, an anti-CTLA-4 antibody, or an anti-PD-L1 antibody.

In one embodiment, the checkpoint inhibitor is PD-1. In one embodiment, the checkpoint inhibitor is PD-L1. In one specific, the checkpoint inhibitor is CTLA-4. In one embodiment, the checkpoint inhibitor is an anti-PD1 antibody. In one embodiment, the checkpoint inhibitor is an anti-PD-L1 antibody. In one embodiment, the checkpoint inhibitor is an anti-CTLA4 antibody.

In one embodiment, one or more genetically engineered bacteria are able to improve anti-tumor activity (e.g., tumor proliferation, size, volume, weight) of the co-administered checkpoint inhibitors (e.g., anti-PD-1, anti-PD-L1 and/or anti-CTLA-4), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a checkpoint inhibitor therapy alone under the same conditions or as compared to a bacteria of the same subtype alone, under the same conditions. In one embodiment, one or more bacteria are able to improve anti-tumor activity (e.g., tumor proliferation, size, volume, weight) of the co-administered checkpoint inhibitors (e.g., anti-PD-1, anti-PD-L1 and/or anti-CTLA-4), e.g., 1.0-1.2-fold, 1.2-1.4-fold, 1.4-1.6-fold, 1.6-1.8-fold, 1.8-2-fold, or two-fold more or more as compared to a checkpoint inhibitor therapy alone under the same conditions or as compared to a bacteria of the same subtype alone, under the same conditions. In one embodiment, one or more bacteria are able to improve anti-tumor activity (e.g., tumor proliferation, size, volume, weight) of the co-administered checkpoint inhibitor (e.g., anti-PD-1, anti-PD-L1 and/or anti-CTLA-4), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a checkpoint inhibitor therapy alone under the same conditions, or as compared to a bacteria of the same subtype alone, under the same conditions.

In one embodiment, the bacteria producing arginine and/or consuming ammonia are able to improve anti-tumor activity (e.g., tumor proliferation, size, volume, weight) of the co-administered checkpoint inhibitors (e.g., PD-1, PD-L1 and/or CTLA-4), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a checkpoint inhibitor therapy alone under the same conditions or as compared to a bacteria of the same subtype alone, under the same conditions. In one embodiment, the bacteria that produce arginine and/or consume ammonia are able to improve anti-tumor activity (e.g., tumor proliferation, size, volume, weight) of the co-administered checkpoint inhibitors (e.g., PD-1, PD-L1 and/or CTLA-4), e.g., 1.0-1.2-fold, 1.2-1.4-fold, 1.4-1.6-fold, 1.6-1.8-fold, 1.8-2-fold, or two-fold more or more as compared to a checkpoint inhibitor alone under the same conditions or as compared to a bacteria of the same subtype alone under the same conditions. In one embodiment, the bacteria that produce arginine and/or consume ammonia are able to improve anti-tumor activity (e.g., tumor proliferation, size, volume, weight) of the co-administered checkpoint inhibitor (e.g., PD-1, PD-L1 and/or CTLA-4), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to the checkpoint inhibitor alone under the same conditions or as compared to a bacteria of the same subtype alone under the same conditions.

In any of these bacteria and checkpoint inhibitor combination embodiments, the synergistic combination is capable of reducing tumor cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to the bacteria alone or the checkpoint inhibitor alone. In any of these combination embodiments, combination is capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to the bacteria alone or the checkpoint inhibitor alone. In any of these combination embodiments, combination is capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to the bacteria alone or the checkpoint inhibitor alone. In any of these combination embodiments, the combination is capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to the bacteria alone or the checkpoint inhibitor alone. In any of these combination embodiments, the combination is capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to the bacteria alone or the checkpoint inhibitor alone. In another embodiment, the tumor is colonized by at least about 35,000 CD4⁺ T-cells per gram of tumor tissue. In one embodiment, the tumor is colonized by at least about 30,000 CD4⁺ T-cells per gram of tumor tissue. In one embodiment, the tumor is colonized by at least about 25,000 CD4⁺ T-cells per gram of tumor tissue. In one embodiment, the tumor is colonized by at least about 20,000 CD4⁺ T-cells per gram of tumor tissue. In one embodiment, the tumor is colonized by at least about 15,000 CD4⁺ T-cells per gram of tumor tissue. In one embodiment, the tumor is colonized by at least about 10,000 CD4⁺ T-cells per gram of tumor tissue. In one embodiment, the tumor is colonized by at least about 5,000 CD4⁺ T-cells per gram of tumor tissue.

In another embodiment, the tumor is colonized by at least about 35,000 CD8⁺ T-cells per gram of tumor tissue. In one embodiment, the tumor is colonized by at least about 30,000 CD8⁺ T-cells per gram of tumor tissue. In one embodiment, the tumor is colonized by at least about 25,000 CD8⁺ T-cells per gram of tumor tissue. In one embodiment, the tumor is colonized by at least about 20,000 CD8⁺ T-cells per gram of tumor tissue. In one embodiment, the tumor is colonized by at least about 15,000 CD8⁺ T-cells per gram of tumor tissue. In one embodiment, the tumor is colonized by at least about 10,000 CD8⁺ T-cells per gram of tumor tissue. In one embodiment, the tumor is colonized by at least about 5,000 CD8⁺ T-cells per gram of tumor tissue.

In another embodiment, the anti-tumor activity of the checkpoint inhibitor is increased, resulting in a reduction in tumor volume of at least about 3000 mm³ after administration, as compared to administering the checkpoint inhibitor alone, or administering the modified bacterium alone, to a subject. In one embodiment, the anti-tumor activity of the checkpoint inhibitor is increased, resulting in a reduction in tumor volume of at least about 2500 mm³ after administration, as compared to administering the checkpoint inhibitor alone, or administering the modified bacterium alone, to a subject. In one embodiment, the anti-tumor activity of the checkpoint inhibitor is increased, resulting in a reduction in tumor volume of at least about 2000 mm³ after administration, as compared to administering the checkpoint inhibitor alone, or administering the modified bacterium alone, to a subject. In one embodiment, the anti-tumor activity of the checkpoint inhibitor is increased, resulting in a reduction in tumor volume of at least about 1500 mm³ after administration, as compared to administering the checkpoint inhibitor alone, or administering the modified bacterium alone, to a subject. In one embodiment, the anti-tumor activity of the checkpoint inhibitor is increased, resulting in a reduction in tumor volume of at least about 1000 mm³ after administration, as compared to administering the checkpoint inhibitor alone, or administering the modified bacterium alone, to a subject. In one embodiment, the anti-tumor activity of the checkpoint inhibitor is increased, resulting in a reduction in tumor volume of at least about 500 mm³ after administration, as compared to administering the checkpoint inhibitor alone, or administering the modified bacterium alone, to a subject. In one embodiment, the anti-tumor activity of the checkpoint inhibitor is increased, resulting in a reduction in tumor volume of at least about 100 mm³ after administration, as compared to administering the checkpoint inhibitor alone, or administering the modified bacterium alone, to a subject.

### Pharmaceutical Compositions and Administration

Pharmaceutical compositions comprising the bacteria and checkpoint inhibitor(s) may be used to treat, manage, ameliorate, and/or prevent cancer. Pharmaceutical compositions may be used alone or in combination with prophylactic agents, therapeutic agents, and/or pharmaceutically acceptable carriers.

The pharmaceutical compositions may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In one embodiment, the pharmaceutical compositions are subjected to tableting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The compositions may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, intratumoral, peritumor, immediate-release, pulsatile-release, delayed-release, or sustained release). Suitable dosage amounts for the bacteria may range from about 10⁴ to 10¹² bacteria. The composition may be administered once or more daily, weekly, or monthly.

The bacteria and/or checkpoint inhibitor may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In one embodiment, the bacteria may be formulated in a solution of sodium bicarbonate, e.g., 1 molar solution of sodium bicarbonate (to buffer an acidic cellular environment, such as the stomach, for example).

The compositions may be administered intravenously, e.g., by infusion or injection. Alternatively, the compositions may be administered intratumorally and/or peritumorally. In other embodiments, the compositions may be administered intra-arterially, intramuscularly, or intraperitoneally. In one embodiment, the bacteria colonize about 20%, 30%, 40%, 50%, 60%, 70%, 80%, 90% or more of the tumor. In one embodiment, the bacteria and checkpoint inhibitor are co-administered with a PEGylated form of rHuPH20 (PEGPH20) or other agent in order to destroy the tumor septae in order to enhance penetration of the tumor capsule, collagen, and/or stroma.

The microorganisms and checkpoint inhibitor of the disclosure may be administered via intratumoral injection. Intratumoral injection may elicit a potent localized inflammatory response as well as an adaptive immune response against tumor cells. In one embodiment, the tumor is injected with an 18-gauge multipronged needle (Quadra-Fuse, Rex Medical). The injection site is aseptically prepared. If available, ultrasound or CT may be used to identify a necrotic region of the tumor for injection. If a necrotic region is not identified, the injection can be directed to the center of the tumor. The needle is inserted once into a predefined region, and dispensed with even pressure. The injection needle is removed slowly, and the injection site is sterilized.

Direct intratumoral injection of the compositions into solid tumors may be advantageous. Using an intravenous injection method, only a small proportion of the bacteria may reach the target tumor. For example, following E. coli Nissle injection into the tail vein of 4T1 tumor-bearing mice, most bacteria (>99%) are quickly cleared from the animals and only a small percentage of the administered bacteria colonize the tumor (Stritzker et al., 2007). In particular, in large animals and human patients, which have relatively large blood volumes and relatively small tumors compared to mice, intratumoral injection may be especially beneficial. Injection directly into the tumor allows the delivery of a higher concentration of therapeutic agent and avoids the toxicity, which can result from systemic administration. In addition, intratumoral injection of bacteria induces robust and localized immune responses within the tumor.

Depending on the location, tumor type, and tumor size, different administration techniques may be used, including but not limited to, cutaneous, subcutaneous, and percutaneous injection, therapeutic endoscopic ultrasonography, or endobronchial intratumor delivery. Prior to the intratumor administration procedures, sedation in combination with a local anesthetic and standard cardiac, pressure, and oxygen monitoring, or full anesthesia of the patient is performed.

For some tumors, percutaneous injection can be employed, which is the least invasive administration method. Ultrasound, computed tomography (CT) or fluoroscopy can be used as guidance to introduce and position the needle. Percutaneous intratumoral injection is for example described for hepatocellular carcinoma in Lencioni et al., 2010. Intratumoral injection of cutaneous, subcutaneous, and nodal tumors is for example described in WO/2014/036412 (Amgen) for late stage melanoma.

Single insertion points or multiple insertion points can be used in percutaneous injection protocols. Using a single insertion point, the solution may be injected percutaneously along multiple tracks, as far as the radial reach of the needle allows. In other embodiments, multiple injection points may be used if the tumor is larger than the radial reach of the needle. The needle can be pulled back without exiting, and redirected as often as necessary until the full dose is injected and dispersed. To maintain sterility, a separate needle is used for each injection. Needle size and length varies depending on the tumor type and size.

In one embodiment, the tumor is injected percutaneously with an 18-gauge multipronged needle (Quadra-Fuse, Rex Medical). The device consists of an 18 gauge puncture needle 20 cm in length. The needle has three retractable prongs, each with four terminal side holes and a connector with extension tubing clamp. The prongs are deployed from the lateral wall of the needle. The needle can be introduced percutaneously into the center of the tumor and can be positioned at the deepest margin of the tumor. The prongs are deployed to the margins of the tumor. The prongs are deployed at maximum length and then are retracted at defined intervals. Optionally, one or more rotation-injection-rotation maneuvers can be performed, in which the prongs are retracted, the needle is rotated by a 60 degrees, which is followed by repeat deployment of the prongs and additional injection.

Therapeutic endoscopic ultrasonography (EUS) is employed to overcome the anatomical constraints inherent in gaining access to certain other tumors (Shirley et al., 2013). EUS-guided fine needle injection (EUS-FNI) has been successfully used for antitumor therapies for the treatment of head and neck, esophageal, pancreatic, hepatic, and adrenal masses (Verna et al, 2008). EUS-FNI has been extensively used for pancreatic cancer injections. Fine-needle injection requires the use of the curvilinear echoendoscope. The esophagus is carefully intubated and the echoendoscope is passed into the stomach and duodenum where the pancreatic examination occurs, and the target tumor is identified. The largest plane is measured to estimate the tumor volume and to calculate the injection volume. The appropriate volume is drawn into a syringe. A primed 22-gauge fine needle aspiration (FNA) needle is passed into the working channel of the echoendoscope. Under ultrasound guidance, the needle is passed into the tumor. Depending on the size of the tumor, administration can be performed by dividing the tumor into sections and then injecting the corresponding fractions of the volume into each section. Use of an installed endoscopic ultrasound processor with Doppler technology assures there are no arterial or venous structures that may interfere with the needle passage into the tumor (Shirley et al., 2013). In one embodiment, ‘multiple injectable needle’ (MIN) for EUS-FNI can be used to improvement the injection distribution to the tumor in comparison with straight-type needles (Ohara et al., 2013).

Intratumoral administration for lung cancer, such as non-small cell lung cancer, can be achieved through endobronchial intratumor delivery methods, as described in Celikoglu et al., 2008. Bronchoscopy (trans-nasal or oral) is conducted to visualize the lesion to be treated. The tumor volume can be estimated visually from visible length-width height measurements over the bronchial surface. The needle device is then introduced through the working channel of the bronchoscope. The needle catheter, which consists of a metallic needle attached to a plastic catheter, is placed within a sheath to prevent damage by the needle to the working channel during advancement. The needle size and length varies and is determined according to tumor type and size of the tumor. Needles made from plastic are less rigid than metal needles and are ideal, since they can be passed around sharper bends in the working channel. The needle is inserted into the lesion and the bacteria are in injected. Needles are inserted repeatedly at several insertion points until the tumor mass is completely perfused. After each injection, the needle is withdrawn entirely from the tumor and is then embedded at another location. At the end of the bronchoscopic injection session, removal of any necrotic debris caused by the treatment may be removed using mechanical dissection, or other ablation techniques accompanied by irrigation and aspiration.

In one embodiment, the compositions are administrated directly into the tumor using methods, including but not limited to, percutaneous injection, EUS-FNI, or endobronchial intratumor delivery methods. In some cases, other techniques, such as laparoscopic or open surgical techniques are used to access the target tumor, however, these techniques are much more invasive and bring with them much greater morbidity and longer hospital stays.

The dose to be injected is derived from the type and size of the tumor. The dose of a drug or the bacteria is typically lower, e.g., orders of magnitude lower, than a dose for systemic intravenous administration.

The volume injected into each lesion is based on the size of the tumor. To obtain the tumor volume, a measurement of the largest plane can be conducted. The estimated tumor volume can then inform the determination of the injection volume as a percentage of the total volume. For example, an injection volume of approximately 20-40% of the total tumor volume can be used.

For example, as is for example described in WO/2014/036412, for tumors larger than 5 cm in their largest dimension, up to 4 ml can be injected. For tumors between 2.5 and 5 cm in their largest dimension, up to 2 ml can be injected. For tumors between 2.5 and 5 cm in their largest dimension, up to 2 ml can be injected. For tumors between 1.5 and 2.5 cm in their largest dimension, up to 1 ml can be injected. For tumors between 0.5 and 1.5 cm in their largest dimension, up to 0.5 ml can be injected. For tumors equal or small than 0.5 in their largest dimension, up to 0.1 ml can be injected. Alternatively, ultrasound scan can be used to determine the injection volume that can be taken up by the tumor without leakage into surrounding tissue.

In one embodiment, the treatment regimen will include one or more intratumoral administrations. In one embodiment, a treatment regimen will include an initial dose, which followed by at least one subsequent dose. One or more doses can be administered sequentially in two or more cycles.

For example, a first dose may be administered at day 1, and a second dose may be administered after 1, 2, 3, 4, 5, 6, days or 1, 2, 3, or 4 weeks or after a longer interval. Additional doses may be administered after 1, 2, 3, 4, 5, 6, days or after 1, 2, 3, or 4 weeks or longer intervals. In one embodiment, the first and subsequent administrations have the same dosage. In other embodiments, different doses are administered. In one embodiment, more than one dose is administered per day, for example, two, three or more doses can be administered per day.

The routes of administration and dosages described are intended only as a guide. The optimum route of administration and dosage can be readily determined by a skilled practitioner. The dosage may be determined according to various parameters, especially according to the location of the tumor, the size of the tumor, the age, weight and condition of the patient to be treated and the route and method of administration.

In one embodiment, the bacteria is administered via first route, e.g., intratumoral injection, and the at least one checkpoint inhibitor is administered via a second route, e.g., intravenously.

In one embodiment, the compositions of the disclosure may be administered orally. For example, Danino et al showed that orally administered E. coli Nissle is able to colonize liver metastases by crossing the gastrointestinal tract in a mouse model of liver metastases (Danino et al., Programmable probiotics for detection of cancer in urine. Science Translational Medicine, 7 (289): 1-10, the contents of which is herein incorporated by reference in its entirety).

In one embodiment, the composition is delivered intrapleurally. In one embodiment, the composition is delivered subcutaneously. In one embodiment, the composition is delivered intravenously. In one embodiment, the composition is delivered intrapleurally.

In one embodiment, the compositions may be administered intratumorally according to a regimen which requires multiple injections. In one embodiment, the bacteria and checkpoint inhibitor are administered together in each intratumoral injection. In one embodiment, a bacteria strain is injected first and a checkpoint inhibitor is injected at a later timepoint. In other embodiments, a checkpoint inhibitor is injected first, and a bacteria is injected at a later time point. Additional injections, either concurrently or sequentially, can follow.

Tumor types into which the bacteria of the current disclosure are intratumorally delivered include locally advanced and metastatic tumors, including but not limited to, B, T, and NK cell lymphomas, colon and rectal cancers, melanoma, including metastatic melanoma, mycosis fungoides, Merkel carcinoma, liver cancer, including hepatocellular carcinoma and liver metastasis secondary to colorectal cancer, pancreatic cancer, breast cancer, follicular lymphoma, prostate cancer, refractory liver cancer, and Merkel cell carcinoma.

In one embodiment, tumor cell lysis occurs as part of the intratumor injection. As result, tumor antigens may exposed eliciting an anti-tumor response. This exposure may work together with the effector expressed by the bacteria to enhance the anti-tumor effect. In one embodiment, tumor cell lysis does not occur as part of the intratumor injection.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

### Methods of Treatment

Disclosed herein are methods of treating cancer. In one embodiment, the disclosure provides methods for reducing, ameliorating, or eliminating one or more symptom(s) associated with cancer. In one embodiment, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, liver cancer, lung cancer, malignant mesothelioma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macroglobulinemia, and Wilms tumor.

In one embodiment, the symptom(s) associated thereof include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration.

The method may comprise preparing a pharmaceutical composition with at least one species, strain, or subtype of bacteria and checkpoint inhibitor described herein, and administering the pharmaceutical composition to a subject in a therapeutically effective amount. The composition may be administered locally, e.g., intratumorally or peritumorally into a tissue or supplying vessel, or systemically, e.g., intravenously by infusion or injection. In one embodiment, the compositions are administered intravenously, intratumorally, intra-arterially, intramuscularly, intraperitoneally, orally, or topically. In one embodiment, the compositions are administered intravenously, i.e., systemically.

In certain embodiments, administering the pharmaceutical composition to the subject reduces cell proliferation, tumor growth, and/or tumor volume in a subject. In one embodiment, the methods of the present disclosure may reduce cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to levels in an untreated or control subject. In one embodiment, reduction is measured by comparing cell proliferation, tumor growth, and/or tumor volume in a subject before and after administration of the pharmaceutical composition. In one embodiment, the method of treating or ameliorating a cancer in a subject allows one or more symptoms of the cancer to improve by at least about 10%, 20%, 30%, 40%, 50%, 60%, 70%, 80%, 90%, 95%, or more.

Before, during, and after the administration of the pharmaceutical composition, cancerous cells and/or biomarkers in a subject may be measured in a biological sample, such as blood, serum, plasma, urine, peritoneal fluid, and/or a biopsy from a tissue or organ. In one embodiment, the methods may include administration of the compositions to reduce tumor volume in a subject to an undetectable size, or to less than about 1%, 2%, 5%, 10%, 20%, 25%, 30%, 40%, 50%, 60%, 70%, 75%, 80%, or 90% of the subject's tumor volume prior to treatment. In other embodiments, the methods may include administration of the compositions to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

In one embodiment, the tumor volume is decreased to less than about 500 mm³ after 30 days of treatment. In one embodiment, the tumor volume is decreased to less than about 400 mm³ after 30 days of treatment. In one embodiment, the tumor volume is decreased to less than about 300 mm³ after 30 days of treatment. In one embodiment, the tumor volume is decreased to less than about 200 mm³ after 30 days of treatment. In one embodiment, the tumor volume is decreased to less than about 100 mm³ after 30 days of treatment.

In one embodiment, tumor growth is inhibited for at least 5 days after treatment, at least 10 days after treatment, at least 14 days after treatment, at least 21 days after treatment, at least 30 days after treatment, or at least two months after treatment.

In another embodiment, arginine levels in the TME are increased, and ammonia levels in the TME are decreased. In one embodiment, the arginine levels in the TME are increased to at least about 30 μg of arginine per gram of tumor. In one embodiment, the arginine levels in the TME are increased to at least about 25 μg of arginine per gram of tumor. In one embodiment, the arginine levels in the TME are increased to at least about 20 μg of arginine per gram of tumor. In one embodiment, the arginine levels in the TME are increased to at least about 15 μg of arginine per gram of tumor. In one embodiment, the arginine levels in the TME are increased to at least about 10 μg of arginine per gram of tumor. In one embodiment, the arginine levels in the TME are increased to at least about 5 μg of arginine per gram of tumor. In one embodiment, the arginine levels in the TME are increased to at least about 3 μg of arginine per gram of tumor.

In one embodiment, the ammonia levels in a tumor microenvironment (TME) are reduced in comparison to an endogenous amount.

In one embodiment, at least 15% of subjects in the population of subjects exhibit complete eradication of the tumor. In one embodiment, at least 20% of subjects in the population of subjects exhibit complete eradication of the tumor. In one embodiment, at least 25% of subjects in the population of subjects exhibit complete eradication of the tumor. In one embodiment, at least 30% of subjects in the population of subjects exhibit complete eradication of the tumor. In one embodiment, at least 35% of subjects in the population of subjects exhibit complete eradication of the tumor. In one embodiment, at least 40% of subjects in the population of subjects exhibit complete eradication of the tumor. In one embodiment, at least 45% of subjects in the population of subjects exhibit complete eradication of the tumor. In one embodiment, at least 50% of subjects in the population of subjects exhibit complete eradication of the tumor. In one embodiment, at least 55% of subjects in the population of subjects exhibit complete eradication of the tumor. In one embodiment, at least 60% of subjects in the population of subjects exhibit complete eradication of the tumor. In one embodiment, at least 65% of subjects in the population of subjects exhibit complete eradication of the tumor. In one embodiment, at least 70% of subjects in the population of subjects exhibit complete eradication of the tumor. In one embodiment, at least 75% of subjects in the population of subjects exhibit complete eradication of the tumor.

In one embodiment, at least 15% of subjects in the population of subjects exhibit partial eradication of the tumor. In one embodiment, at least 20% of subjects in the population of subjects exhibit partial eradication of the tumor. In one embodiment, at least 25% of subjects in the population of subjects exhibit partial eradication of the tumor. In one embodiment, at least 30% of subjects in the population of subjects exhibit partial eradication of the tumor. In one embodiment, at least 35% of subjects in the population of subjects exhibit partial eradication of the tumor. In one embodiment, at least 40% of subjects in the population of subjects exhibit partial eradication of the tumor. In one embodiment, at least 45% of subjects in the population of subjects exhibit partial eradication of the tumor. In one embodiment, at least 50% of subjects in the population of subjects exhibit partial eradication of the tumor. In one embodiment, at least 55% of subjects in the population of subjects exhibit partial eradication of the tumor. In one embodiment, at least 60% of subjects in the population of subjects exhibit partial eradication of the tumor. In one embodiment, at least 65% of subjects in the population of subjects exhibit partial eradication of the tumor. In one embodiment, at least 70% of subjects in the population of subjects exhibit partial eradication of the tumor. In one embodiment, at least 75% of subjects in the population of subjects exhibit partial eradication of the tumor.

Response patterns may be different than for traditional cytotoxic therapies. For example, tumors treated with immune-based therapies may enlarge before they regress, and/or new lesions may appear (Agarwala et al., 2015). Increased tumor size may be due to heavy infiltration with lymphocytes and macrophages that are normally not present in tumor tissue. Additionally, response times may be slower than response times associated with standard therapies, e.g., cytotoxic therapies.

The bacteria may be destroyed, e.g., by defense factors in tissues or blood serum (Sonnenborn et al., 2009), or by activation of a kill switch, several hours or days after administration. Thus, the pharmaceutical composition may be re-administered at a therapeutically effective dose and frequency. In alternate embodiments, the bacteria are not destroyed within hours or days after administration and may propagate in the tumor and colonize the tumor.

The pharmaceutical composition may be administered alone or in combination with one or more additional therapeutic agents, e.g., a chemotherapeutic drug or a checkpoint inhibitor, e.g., as described herein and known in the art. An important consideration in selecting the one or more additional therapeutic agents is that the agent(s) should be compatible with the bacteria of the disclosure, e.g., the agent(s) must not kill the bacteria. In some studies, the efficacy of anticancer immunotherapy, e.g., CTLA-4 or PD-1 inhibitors, requires the presence of particular bacterial strains in the microbiome (Ilda et al., 2013; Vetizou et al., 2015; Sivan et al., 2015). In one embodiment, the pharmaceutical composition comprising the bacteria augments the effect of a checkpoint inhibitor or a chemotherapeutic agent, e.g., allowing lowering of a the dose of systemically administrated chemotherapeutic or immunotherapeutic agents. In one embodiment, the pharmaceutical composition is administered with one or more commensal or probiotic bacteria, e.g., Bifidobacterium or Bacteroides.

In certain embodiments, administering the composition(s) to the subject reduces ammonia levels and/or increases arginine levels in a subject. Before, during, and after the administration of the composition, ammonia and/or arginine concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal.

### Animal Models

The synergistic compositions disclosed herein may be evaluated in vivo, e.g., in an animal model. Any suitable animal model of a disease or condition associated with cancer may be used, e.g., a tumor syngeneic or xenograft mouse models (see, e.g., Yu et al., 2015). The arginine-producing and/or ammonia-consuming bacteria and the checkpoint inhibitor may be administered to the animal systemically or locally, e.g., via oral administration (gavage), intravenous, or subcutaneous injection or via intratumoral injection, and treatment efficacy determined, e.g., by measuring tumor volume.

Non-limiting examples of animal models include mouse models, as described in Dang et al., 2001, Heap et al., 2014 and Danino et al., 2015).

Pre-clinical mouse models determine which immunotherapies and combination immunotherapies will generate the optimal therapeutic index (maximal anti-tumor efficacy and minimal immune related adverse events (irAEs)) in different cancers.

Implantation of cultured cells derived from various human cancer cell types or a patient's tumor mass into mouse tissue sites has been widely used for generations of cancer mouse models (xenograft modeling). In xenograft modeling, human tumors or cell lines are implanted either subcutaneously or orthotopically into immune-compromised host animals (e.g., nude or SCID mice) to avoid graft rejection. Because the original human tumor microenvironment is not recapitulated in such models, the activity of anti-cancer agents that target checkpoint inhibitors may not be accurately measured in these models, making mouse models with an intact immune system more desirable.

Accordingly, implantation of murine cancer cells in a syngeneic immunocompetent host (allograft) are used to generate mouse models with tumor tissues derived from the same genetic background as a given mouse strain. In syngeneic models, the host immune system is normal, which may more closely represent the real life situation of the tumor's micro-environment. The tumor cells or cancer cell lines are implanted either subcutaneously or orthotopically into the syngeneic immunocompetent host animal (e.g., mouse). Representative murine tumor cell lines, which can be used in syngeneic mouse models for immune checkpoint benchmarking include, but are not limited to the cell lines listed in International Patent Application PCT/US2017/013072, filed Jan. 11, 2017, published as WO2017/123675, the contents of which is herein incorporated by reference in its entirety.

For tumors derived from certain cell lines, ovalbumin can be added to further stimulate the immune response, thereby increasing the response baseline level. Examples of mouse strains that can be used in syngeneic mouse models, depending on the cell line include C57BL/6, FVB/N, Balb/c, C3H, HeJ, C3H/HeJ, NOD/ShiLT, A/J, 129S1/SvlmJ, NOD. Additionally, several further genetically engineered mouse strains have been reported to mimic human tumorigenesis at both molecular and histologic levels. These genetically engineered mouse models also provide excellent tools to the field and additionally, the cancer cell lines derived from the invasive tumors developed in these models are also good resources for cell lines for syngeneic tumor models Examples of genetically engineered strains are provided in in International Patent Application PCT/US2017/013072, filed Jan. 11, 2017, published as WO2017/123675, the contents of which is herein incorporated by reference in its entirety.

Often potential therapeutic molecules which interact with human checkpoint inhibitors and stimulate human immune system and do not detect their murine counterparts and vice versa. In studying therapeutic molecules, it is necessary to take this in consideration. More recently, “humanized” mouse models have been developed, in which immunodeficient mice are reconstituted with a human immune system, and which have helped overcome issues relating to the differences between the mouse and human immune systems, allowing the in vivo study of human immunity. Severely immunodeficient mice which combine the IL2receptor null and the severe combined immune deficiency mutation (scid) (NOD-scid IL2Rgnull mice) lack mature T cells, B cells, or functional NK cells, and are deficient in cytokine signaling. These mice can be engrafted with human hematopoietic stem cells and peripheral-blood mononuclear cells. CD34+ hematopoietic stem cells (hu-CD34) are injected into the immune deficient mice, resulting in multi-lineage engraftment of human immune cell populations including very good T cell maturation and function for long-term studies. This model has a research span of 12 months with a functional human immune system displaying T-cell dependent inflammatory responses with no donor cell immune reactivity towards the host. Patient derived xenografts can readily be implanted in these models and the effects of immune modulatory agents studied in an in vivo setting more reflective of the human tumor microenvironment (both immune and non-immune cell-based) (Baia et al., 2015). Human cell lines of interest for use in the humanized mouse models include but are not limited to HCT-116 and HT-29 colon cancer cell lines.

A rat F98 glioma model and the utility of spontaneous canine tumors, as described in Roberts et al 2014, the contents of each of which are herein incorporated by reference in their entireties. Locally invasive tumors generated by implantation of F98 rat glioma cells engineered to express luciferase were intratumorally injected with C. novyi-NT spores, resulting in germination and a rapid fall in luciferase activity. C. novyi-NT germination was demonstrated by the appearance of vegetative forms of the bacterium. In these studies, C. novyi-NT precisely honed to the tumor sparing neighboring cells.

Canine soft tissue sarcomas for example are common in many breeds and have clinical, histopathological, and genetically features similar to those in humans (Roberts et al, 2014; Staedtke et al., 2015), in particular, in terms of genetic alterations and spectrum of mutations. Roberts et al. conducted a study in dogs, in which C. novyi-NT spores were intratumorally injected (1×10⁸ C. novyi-NT spores) into spontaneously occurring solid tumors in one to 4 treatment cycles and followed for 90 days. A potent inflammatory response was observed, indicating that the intratumoral injections mounted an innate immune response.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

Construction of plasmids encoding ammonia consuming circuits, including circuits comprising AArgR, ArgA^{fbr}, and/or AThyA are inter alia described in the Examples of WO2017139697 and US20160333326, the contents of which are herein incorporated by reference in their entirety. A Functional Assay Demonstrating that the Recombinant Bacterial Cells disclosed herein consume ammonia and produce arginine is inter alia described in the Examples of WO2017139697 and US20160333326, the contents of which is herein incorporated by reference in its entirety. The in vitro activity of various strains (i.e., including AArgR and ArgA^{fbr} plus or minus AThyA) is described in the Examples of WO2017139697 and US20160333326. In vivo activity assays which may be used to determine in vivo efficacy for any of the strains described herein, are described in Examples of WO2017139697 and US20160333326, the contents of which is herein incorporated by reference in its entirety. Integration of constructs into the genome, e.g., using lambda red recombination is also described in WO2017139697 and US20160333326.

### Example 1. Introduction and Evaluation of Therapeutic Efficacy of L-Arginine in Combination with a Checkpoint Inhibitor

Immune checkpoint blockade is proving to be an effective therapeutic approach to treat cancer. However, the remarkable responses to antibodies that block the PD-1 or CTLA-4 signaling pathways are currently limited to a minority of patients, highlighting the need to develop novel and combinatorial approaches.

Tumors create a suppressive metabolic microenvironment that contributes to ineffective anti-tumor immunity. For example, glucose and arginine are often scarce in the tumor microenvironment (TME), which hampers the functionality of T cells. Thus, a potential strategy to improve anti-tumor T cell responses is to design interventions that alter the metabolic milieu in tumors in favor of T cell function.

The amino acid L-arginine plays an important role in the regulation of the immune system. A meta-analysis of 11 clinical trials revealed that dietary supplementation of L-arginine increases the CD4⁺ T-cell proliferative response and reduces the incidence of infectious complications. In tumors, L-arginine levels are typically low because tumor-associated macrophages, myeloid-derived suppressor cells and tumor cells can express high levels of arginase-1, which breaks down L-arginine and dampens the anti-tumor activity of T cells. It has previously been shown that increasing intracellular L-arginine levels enhances mitochondrial function of T cells and endows them with a higher survival capacity and anti-tumor functionality. In agreement with this, oral administration of L-arginine to tumor-bearing mice enhances anti-tumor T cell responses and slows down the progression of melanoma, osteosarcoma, colon and breast cancer. However, to achieve the desired effect, relatively high doses of L-arginine have to be administered to mice. In comparison, a patient weighing 75 kg would need to ingest 150 g of L-arginine daily, which is nearly impracticable and highlights the need to develop alternative means to locally increase L-arginine concentrations in tumors.

Synthetic biology applies engineering tools and principles to living systems, and allows the rational rewiring of cells for the execution of pre-defined biological activities upon sensing and integration of environmental inputs. Because bacteria can survive and thrive in tumors, a non-pathogenic strain of E. coli (E. coli Nissle 1917) was used as an intra-tumor, synthetic biology-based cellular therapy to produce high local concentrations of arginine in the TME. The engineering design was based on the observation that cancer cells produce high levels of ammonia as a metabolic byproduct and therefore a genetic design that results in the efficient utilization of ammonia as substrate for conversion into arginine was designed. The engineered strain is able to convert ammonia into arginine efficiently in vitro and is able to colonize tumors and locally produce high levels of arginine in the TME that results in robust infiltration of T cells and profound efficacy in combination with anti-PD-L1 immunotherapy.

To begin, in vivo activity and efficacy of the combination of an L-arginine solution with an checkpoint inhibitor, compared to the L-arginine solution alone and a PBS control, was determined in MC38 tumors.

MC38 murine colon adenocarcinoma cells were cultured in RPMI 1640 plus 10% heat-inactivated FCS, 1 mM Na-Pyruvate and 50 μM β-mercaptoethanol. Female C57BL/6 mice between the ages of 5 to 10 weeks were implanted with the MC38 cells. Before injection into mice, cells were trypsinized and washed twice in PBS. Then, 5×10⁵ cells were subcutaneously injected in the dorsal region of C57BL/6 WT mice. The size of tumors was measured in a blinded fashion using calipers. Ten days post injection, when tumors started to be visible and palpable, mice bearing small, visible MC38 tumors were randomly assigned to four groups. The control group received intraperitoneal (i.p.) injections of PBS and oral gavage of water while the other groups received daily an L-arginine solution (2 mg/g of body weight) by oral gavage either alone or in combination with αPD-L1 antibody (i.p). Age-matched control mice were treated with PBS i.p. and fed with H₂O. Mice were euthanatized at day 20 after tumor injection. Tumors were collected, digested and tumor-infiltrating lymphocytes (TILs) were analyzed by flow cytometry.

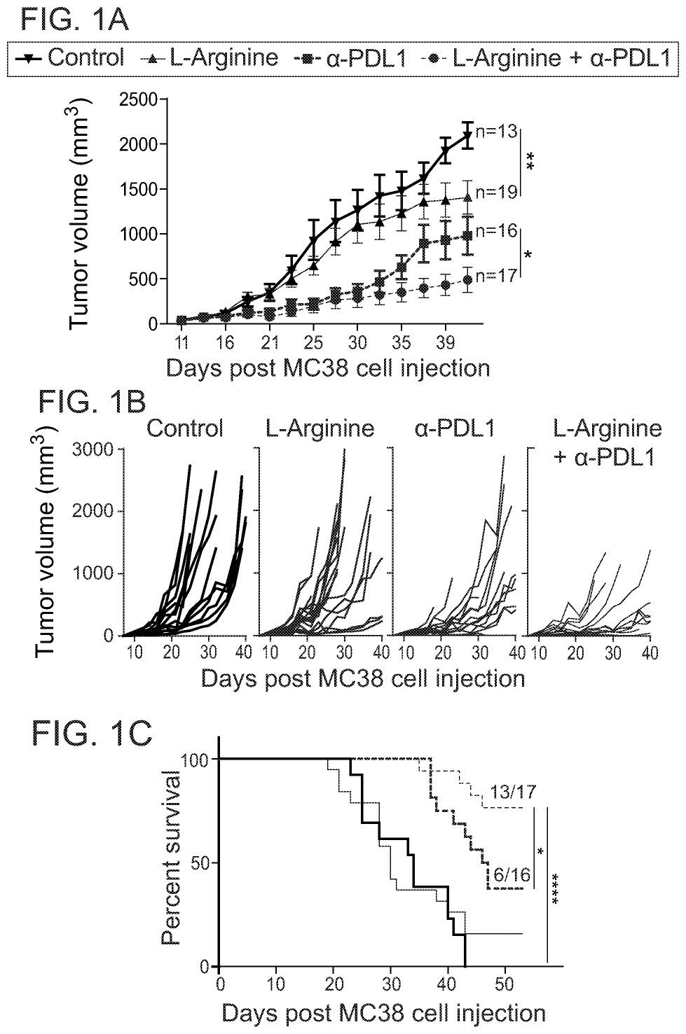

Mice that received daily oral L-arginine administration exhibited slightly reduced MC38 tumor growth, and mice that received αPD-L1 antibodies exhibited a significantly reduced tumor growth as compared to the control group (FIGS. 1A and 1B). Remarkably, combinatorial administration of L-arginine and αPD-L1 antibodies reduced tumor growth further and significantly prolonged the survival of tumor-bearing mice compared to L-arginine or αPD-L1 treatment alone (FIG. 1C). These data suggest that L-arginine and PD-L1 blockade cooperate to restrict tumor growth and provide an effective combinatorial anti-tumor treatment in mice.

### Example 2. Construction of Arginine-Producing Bacteria

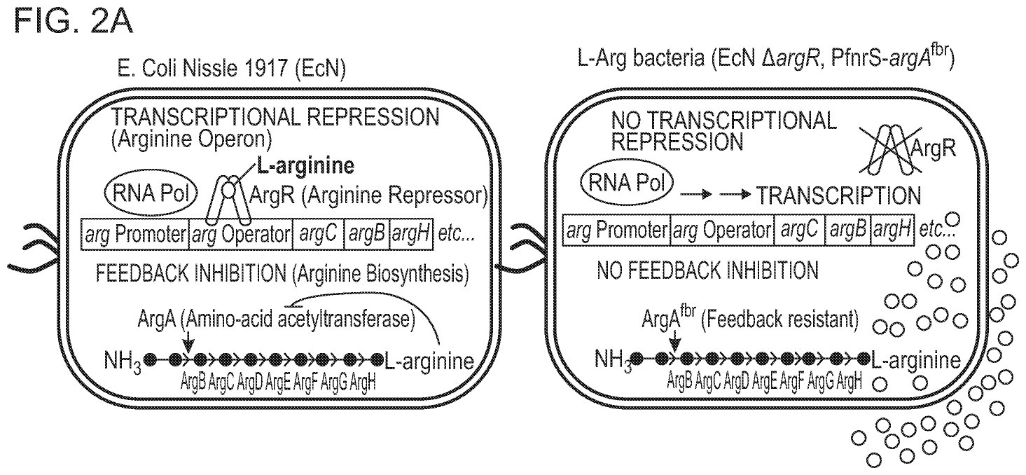

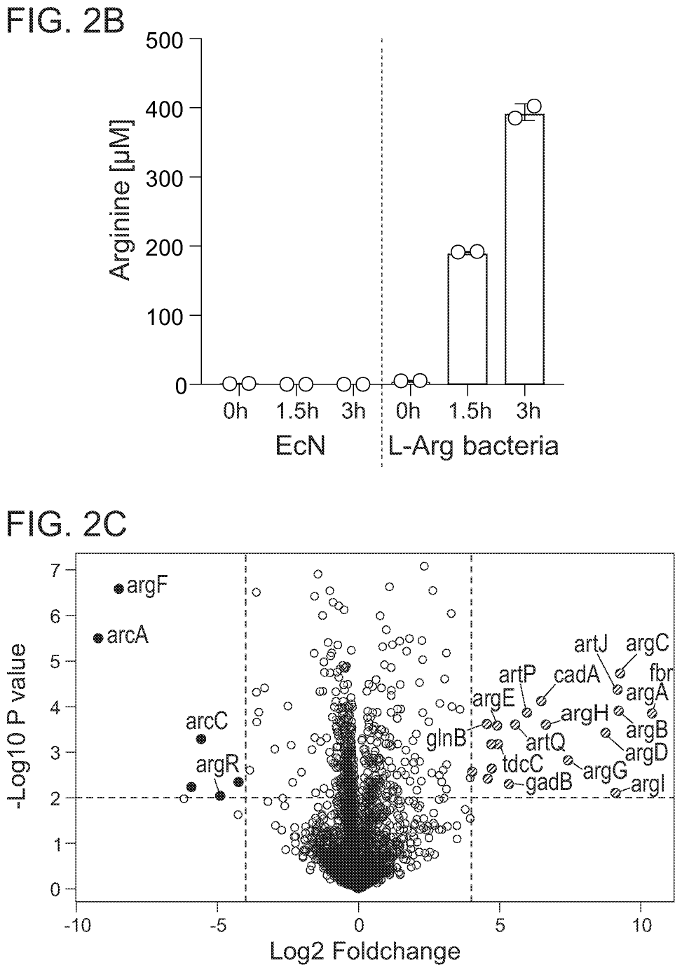

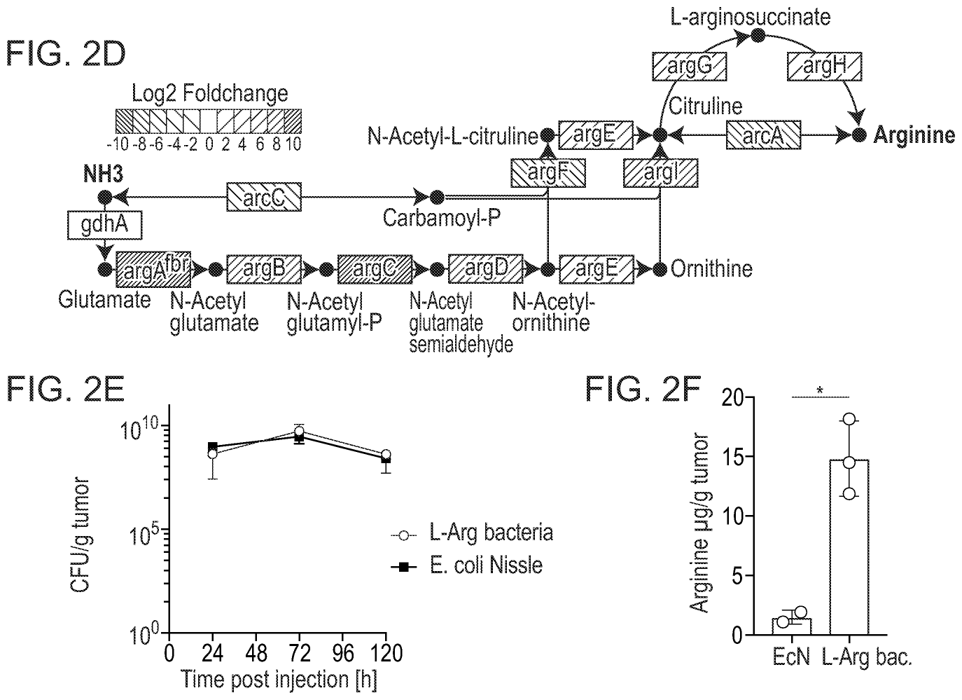

A therapeutic with the ability to continuously produce L-arginine within the tumor micro-environment (TME) was constructed (FIG. 2A). Tumors commonly accumulate high levels of ammonia, a phenomena that has been primarily linked to increased glutaminolysis in proliferating cancer cells. Capitalizing on the availability of ammonia within the TME, the EcN was engineered to efficiently channel ammonia into the synthesis of high levels of arginine under anaerobic conditions found in tumors. Since the ArgR protein negatively regulates the expression of all genes in the arginine biosynthesis pathway, the argR gene was deleted. Additionally, in E. coli the key arginine biosynthesis pathway enzyme N-acetal glutamate synthase (ArgA) is inhibited by high levels of internal L-arginine, thus preventing excess arginine production. To circumvent this, a feedback resistant, dominant mutant of ArgA, designated as ArgA^{fbr}, which is not inhibited by high levels of L-arginine was integrated in the genome. As an additional level of inducible control, the argA^{fbr} gene was placed under the control of the hypoxia-inducible promoter P_{fnrS}. During ex vivo expansion for biomass production, high oxygen levels prevent the expression of fnr-argA^{fbr} whereas the hypoxic conditions within the tumor induces the expression of fnr-argA^{fbr} in vivo. This L-arginine-producing strain is hereafter referred to as L-Arg bacteria.

Specifically, Escherichia coli Nissle 1917 (Nissle) was purchased from the German Collection of Microogranisms and Cell Cultures (DSMZ Braunschweig, E. coli DSM 6601). Two modifications of the native arginine biosynthesis pathway, deletion of argR and insertion of a feedback resistant version of argA (argA^{fbr}), were engineered into the chromosome of Nissle in order to convert ammonia to excess arginine. Control of argA was placed under the oxygen responsive promoter P_{fnrS} to allow for repression of the gene during biomass production and induction in the hypoxic conditions of the tumor microenvironment. One copy of the fnr-argA^{fbr} with chloramphenicol resistant marker was introduced between the malE and malK genes. Both deletion of argR and chromosomal insertion of argA^{fbr} were done by standard E. coli manipulation method such as lambda red recombineering.

Bacterial cultures were grown in LB media supplemented with selective antibiotics (either streptomycin or chloramphenicol) in flasks overnight at 37° C. with shaking at 250 rpm. The overnight cultures were then diluted 100 fold into 100 mL rich media with antibiotics in 1 L baffled flasks. The diluted cultures were expanded by shaking at 250 rpm, 37° C., until reaching an OD₆₀₀˜6. Cells were harvested by centrifugation and washed once with cold PBS. The cells were then resuspended in 15% glycerol+PBS, aliquoted into cryovials and stored at −80° C. until ready for use. Before use cells were thawed on ice, centrifuged at 10,000 rcf for 10 minutes to pellet, glycerol supernatants removed and then rinsed in PBS.

### Example 3. In Vitro L-Arginine Production and Expression of ArgA^{fbr}

L-arginine production in genetically engineered L-Arg bacteria strains was compared to non-engineered ECN. The L-Arg bacteria was cultured with 5 mM ammonia chloride. Specifically, bacterial stocks stored at −80° C. were thawed on ice and diluted 20 fold into 20 mL 2YT media with no antibiotics. Cultures were incubated at 37° C. while shaking at 250 rpm for 2 hours. Cells were pelleted by centrifugation and washed once with PBS. Cell number was measured by cellometer, and bacteria were resuspended into a final concentration of 1×10⁹ cells/mL using M9+0.5% glucose+5 mM NH₄Cl media into 96 deepwell plates. Representative samples of 0 hours were taken immediately. The deep well plate was covered by breathable membrane and immediately moved to 37° C. with shaking at 250 rpm. Representative samples were then taken at 1.5 hours and 3 hours after culture. All samples were spun down to pellet cells and supernatants were collected in a new 96 well plate to be analyzed by LC-MS/MS for arginine quantification.

In vitro, when compared to the non-engineered EcN, the L-Arg bacteria produced higher levels of L-arginine and continued to accumulate L-arginine after three hours (FIG. 2B). These input ammonia levels were comparable to those reported in the interstitial fluid of human xenografts in the art. In agreement with its in vitro activity, a proteomic analysis using high-resolution mass spectrometry confirmed that engineered L-Arg bacteria were devoid of the arginine repressor (ArgR) and expressed high levels of ArgA^{fbr} (FIGS. 2C and 2D). In addition, engineering these two critical components of the arginine biosynthesis pathway resulted in significant upregulation of additional enzymes within the pathway (such as ArgB-E and ArgG-I) and several subunits of the arginine transporter (ArtJ, ArtP, ArtQ) as compared to non-engineered EcN that were cultured under the same conditions (FIGS. 2C and 2D).

Only a few enzymes were downregulated including ArcA, whose induction is potentially regulated by ArgR. In general, the genetic manipulations had specific and targeted effects on the proteome and did not globally alter the features of the EcN bacteria (FIG. 2C).

One enzyme of particular interest that observed significant upregulation as provided in FIG. 2C was lysine decarboxylase (CadA), which is involved in lysine degradation. It is noteworthy that both lysine and arginine compete for the same transporters, and lysine is commonly used as a competitive inhibitor to reduce arginine uptake by T-cells. However, the up-regulation of cadA shown in FIG. 2C demonstrates that cadA may enhance lysine degradation in the bacteria and facilitate export of arginine out of the bacteria, and that this process may further facilitate arginine importation into the T-cells.

### Example 4. In Vivo Colonization in MC38 Tumors

MC38 tumor cells were cultured and transferred s.c. into C57BL/6 WT mice. Tumors were allowed to grow for 14 days prior to the treatments. Non-engineered EcN control bacteria and L-Arg producing bacteria. 5×10⁶ control or L-Arg bacteria were injected intratumorally (i.t.) with a 26-gauge needle twice a week from the beginning of the treatments until the end of the experiment. L-Arg bacteria and control bacteria were injected alone or in combination with 200 μg of αPD-L1 mAb i.p.

Following intratumoral injection, L-Arg bacteria colonized MC38 tumors to similar levels as non-engineered EcN and persisted long-term (FIG. 2E). Importantly, homogenates of MC38 tumors colonized with L-Arg bacteria contained significantly higher levels of arginine as compared to tumors treated with non-engineered EcN (FIG. 2F). Collectively, this data demonstrates that EcN can be engineered to produce high levels of L-arginine and that treatment of tumors with L-Arg bacteria represents a unique therapeutic approach to increase local levels of this immunomodulatory metabolite within the tumor.

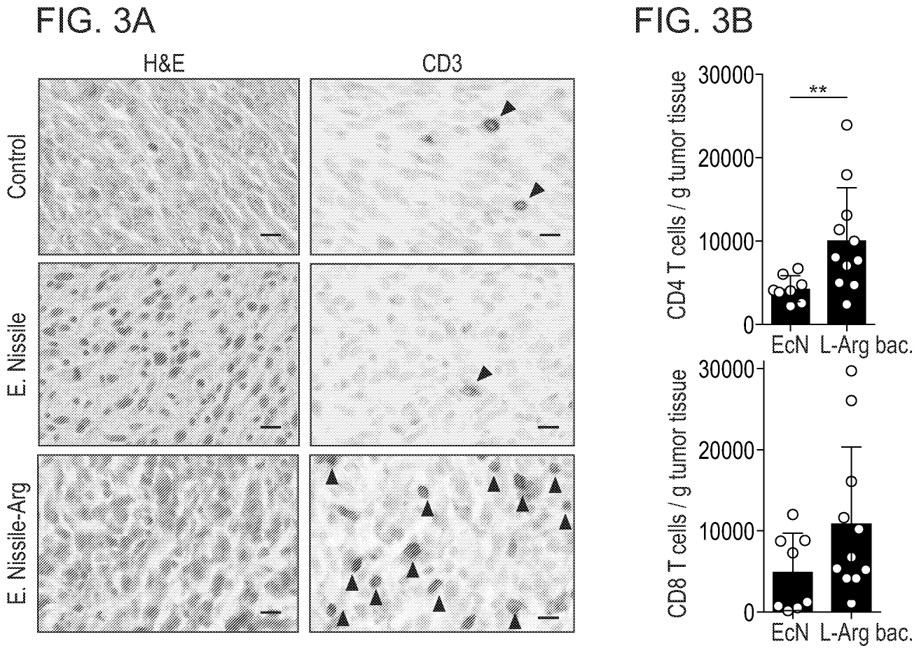

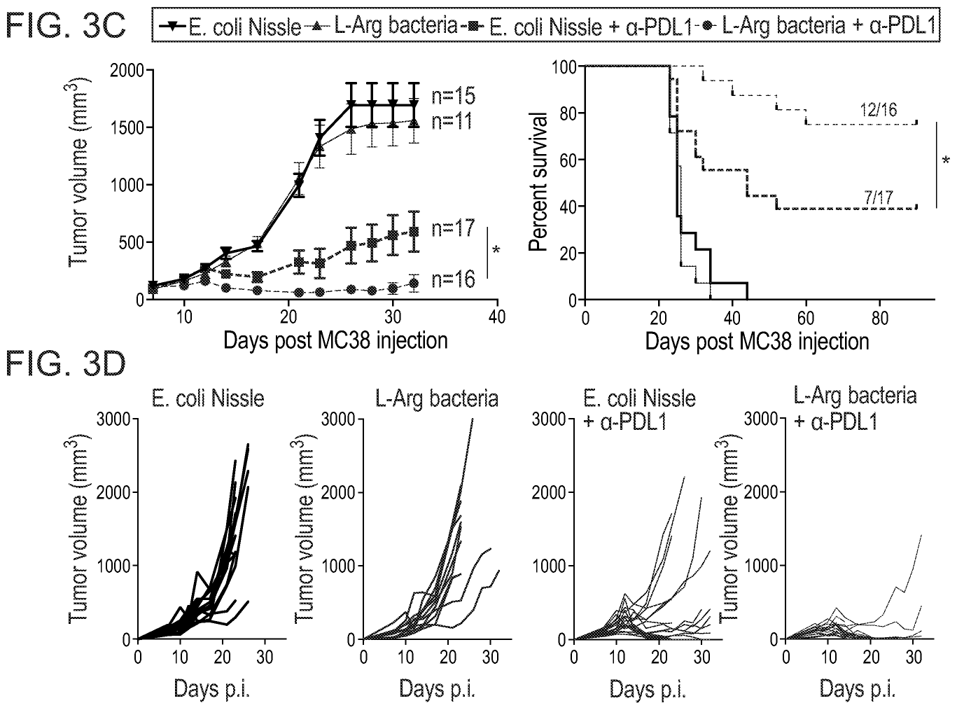

An analysis of tissue sections showed that MC38 tumors colonized with L-Arg bacteria for 72 h contained more tumor-infiltrating T cells than EcN-colonized or untreated tumors (FIG. 3A). To quantify the numbers of tumor-infiltrating CD4⁺ and CD8⁺ T cells, the tumors were dissociated and immune infiltrates were analyzed by flow cytometry. MC38 tumors colonized with L-Arg bacteria contained twice as many CD4⁺ T cells than tumors colonized with non-engineered EcN. A similar trend was observed for CD8⁺ T cells (FIG. 3B).

### Example 5. Arginine-Producing Bacteria Combined with Checkpoint Inhibitors Result in Synergistic Effects Against Tumors

Colonization of MC38 tumors with L-Arg bacteria synergy with blockade of the PD-1 pathway was analyzed. MC38 murine colon adenocarcinoma cells were cultured in RPMI 1640 plus 10% heat-inactivated FCS, 1 mM Na-Pyruvate and 50 μM β-mercaptoethanol. Before injection into mice, cells were trypsinized and washed twice in PBS. Then, 5×10⁵ cells were subcutaneously injected in the dorsal region of C57BL/6 WT mice. The size of tumors was measured in a blinded fashion using calipers. Ten days post injection, when tumors started to be visible and palpable, mice were selected based on tumor size ranging between 10 and 100 mm³. Every second day, mice were injected i.p. with 200 μg of mAb binding PD-L1 (αPD-L1 mAb from Bio X Cell, clone B7-H1), treated daily by oral gavage with an L-arginine solution (2 g/Kg body weight) or received a combination of the two therapies (αPD-L1 mAb+L-Arg feeding) until the end of the experiment. Age-matched control mice were treated with PBS i.p. and fed with H₂O. In another set of experiments, mice were euthanatized at day 20 after tumor injection. Tumors were collected, digested and tumor-infiltrating lymphocytes (TILs) were analyzed by flow cytometry.

Specifically, excised tumors were digested in Collagenase D and DNase I (Roche) and lymphocytes were enriched on a Percoll gradient (Sigma). The following antibodies or streptavidin, conjugated to biotin, FITC, Alexa Fluor (AF) 488, AF647, PE, PE-Cy7, PerCP-Cy5.5, APC, APC-Cy7, Pacific Blue or Pacific Orange were purchased from BD Biosciences, eBioscience, Biolegend, or Molecular Probes: CD3c (clone: 145-2C11), CD4 (RM4-5), CD8 (53-6.7). Dead cells were excluded by staining with DAPI or LIVE/DEAD® Fixable Aqua Dead Cell Stain Kit (Invitrogen™). Flow cytometry was performed on a LSRFortessa (BD Biosciences) and data was evaluated using FlowJo software (TriStar).

For tumor homogenization and CFU count, excised tumors were first weighted and then chopped into small pieces using sterile scissors. Tumor pieces were placed into 2 mL round-bottom tubes (Eppendorf) with 500 μL of PBS and one 5 mm Steel Bead per tumor (Qiagen). Samples were homogenized in a Tissue Lyzer II device (Qiagen) at 300 rpm for 15 min. Serial dilutions of tumor homogenates were plated in LB medium (MP Biomedicals) on Bacto® Agar (BD) plates to which 30 μg/mL chloramphenicol was added for L-Arg bacteria or 300 μg/mL streptomycin (Sigma) for control E. coli Nissle. Plates were incubated 12 h at 37° C. and then bacterial colonies were counted in the dilutions with 30 to 100 colonies.

Strikingly, the combined administration of L-Arg bacteria and PD-L1 blocking antibodies synergistically reduced tumor growth and completely eradicated tumors in 75% of the mice (FIGS. 3C1-3D). These results demonstrate that L-Arg bacteria significantly improve checkpoint inhibitor-based immunotherapy.

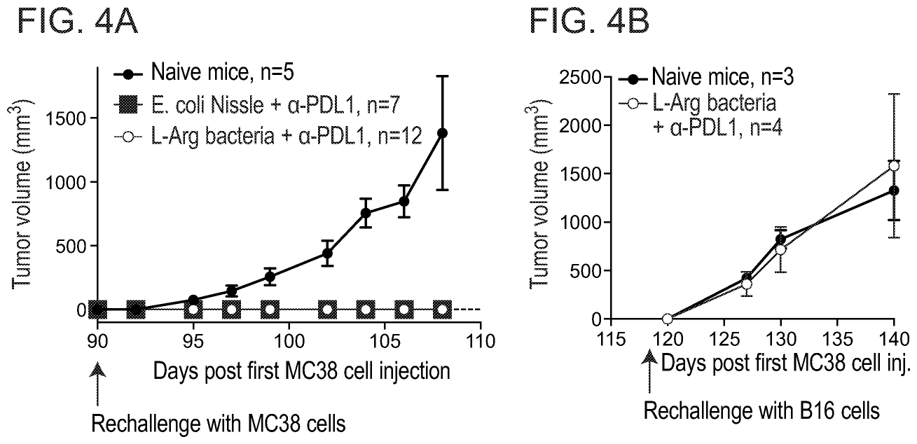

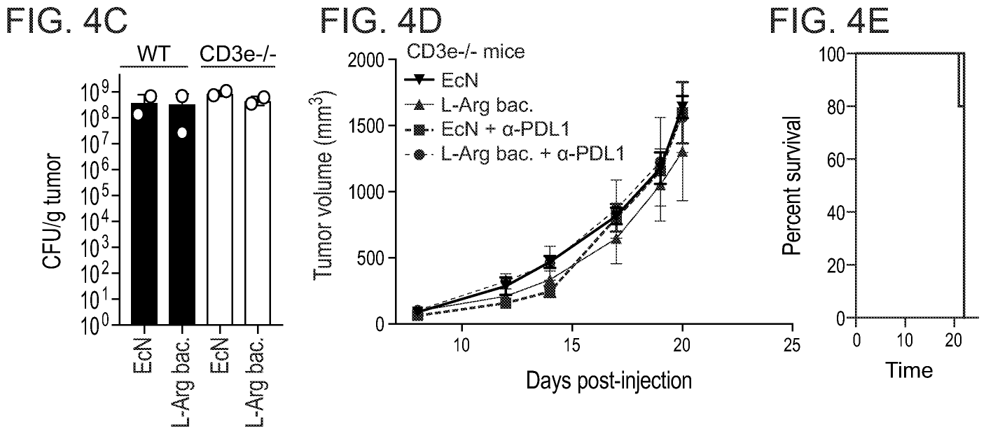

Mice in which tumors regressed completely were re-challenged with a subcutaneous injection of MC38 cells 90 days after the first injection. Unlike in naïve mice, MC38 tumors did not grow in mice that had previously rejected a tumor, indicating that long-term immunological antitumor memory had been induced (FIG. 4A). The antitumor memory was specific to the MC38 tumor model as a subsequent challenge of complete responders with a different tumor cell line (B16) resulted in similar growth kinetics compared to challenged naïve control mice (FIG. 4B). Collectively, these results demonstrate that the combination of L-Arg bacterial treatment with PD-1-based immunotherapy results in substantial tumor control resulting in a 75% complete response rate and formation of tumor-antigen-specific immunological memory in the MC38 model.

### Example 6. T-Cell Dependency on Anti-Tumor Effect

To determine whether L-Arg bacteria inhibit tumor growth directly or whether their anti-tumor activity is T cell-dependent, the effect of L-Arg bacteria was analyzed in combination with PD-L1 blockade in Cd3e^{-/-} mice that lack all T cells but exhibit organized lymphoid organ structures and normal B cell development. Following injection of EcN and L-Arg bacteria, MC38 tumors in Cd3e^{-/-} mice were colonized similar to tumors of wild type mice (FIG. 4C). The growth of MC38 tumors in Cd3e^{-/-} mice that received αPD-L1 antibodies in combination with L-Arg bacteria was comparable to all other groups (FIGS. 4D and 4E). These results indicate that the anti-tumor effect of L-arginine bacteria treatment does not directly impact the growth of MC38 adenocarcinoma cells but depends on T cells and therefore supports the notion of using this synthetic biology approach to increase durable responses in cancer patients undergoing immunotherapy.

### Example 7. LC-MS/MS Analysis of L-Arginine in Bacterial Cultures and Tumor Homogenates

Bacterial supernatant or tumor homogenate was extracted with 4 parts 70% acetonitrile containing 1 μg/mL 15N4,13C6-arginine (Cambridge Isotope Laboratories), vortexed, and centrifuged at 2300 g for 5 minutes at 4° C. Supernatants were diluted 10 fold with 0.1% formic acid prior to analysis. Arginine was separated and detected on a Vanquish UHPLC/TSQ Altis LC-MS/MS system (Thermo Fisher Scientific). Samples were injected (2 μL) and separated on an Accucore aQ 2.6 μm C18, 2.1×100 mm column (Thermo Fisher Scientific). Analytes were eluted using a linear gradient of 95% solution A shifting over to 95% solution B over 2 minutes (Solution A: 0.1% formic acid (FA), 0.02% perfluoropentanoic acid (PFPeA); Solution B: acetonitrile, 0.1% FA, 0.02% PFPeA) at 0.4 mL/min at 40° C. Arginine was detected using selected reaction monitoring (SRM) of compound specific mass transitions in positive electrospray ionization mode (arginine: 175>70; 15N4,13C6-arginine: 185>75). Peaks were integrated and arginine/15N4,13C6-arginine peak area ratios were used to calculate concentrations of the unknowns using a 1/X weighted linear 7 point standard curve from 0.032 to 250 ng/mL using Xcalibur Quan Browser (Thermo Fisher Scientific).

Pre-induced L-Arg bacteria and non-engineered E. coli Nissle were cultured for 3 hours at 37° C. in M9 media+0.5% glucose+5 mM NH₄C1, washed and bacterial pellets were frozen. Bacterial pellets were re-suspended in a ammonium bicarbonate buffer pH=8 (ABC) containing 4% SDS. Then, samples were boiled at 95° C. for 10 min followed by sonication in Bioruptor (15 cycles, 30 s on, 30 s off, high mode). Non-soluble cell debris were spun down at 16,000 g for 10 min and supernatants were precipitated with 5 volumes cold acetone at −80° C. overnight. Precipitated proteins were spun down at 16,000 g for 20 min at 4° C. and pellets was washed twice with ice cold 80% acetone. The pellet was dried in a SpeedVac centrifuge and then re-suspended in ABC buffer containing 8 M urea followed by sonication. Protein amount was determined using a Nanodrop. Proteins were pre-digested with LysC (1:50, w/w) followed by reduction of disulfide bonds with 10 mM DTT and subsequent alkylation with 50 mM iodoacetamide. Then samples were diluted 1:5 with ABC and trypsin (1:50, w/w) was added for overnight digestion at RT. The resulting peptide mixtures were acidified and loaded on C18 StageTips. Peptides were eluted with 80% acetonitrile (ACN), dried using a SpeedVac centrifuge, and resuspended in 2% ACN, 0.1% trifluoroacetic acid (TFA), and 0.5% acetic acid.

Peptides were separated on an EASY-nLC 1200 HPLC system (Thermo Fisher Scientific, Odense) coupled online to a Q Exactive HF mass spectrometer via a nanoelectrospray source (Thermo Fisher Scientific). Peptides were loaded in buffer A (0.5% formic acid) on in house packed columns (75 μm inner diameter, 50 cm length, and 1.9 μm C18 particles from Dr. Maisch GmbH). Peptides were eluted with a non-linear 270 min gradient of 5%-60% buffer B (80% ACN, 0.5% formic acid) at a flow rate of 250 nl/min and a column temperature of 50° C. The Q Exactive HF was operated in a data dependent mode with a survey scan range of 300-1750 m/z and a resolution of 60′000 at ink 200.

MaxQuant software (version 1.5.3.54) was used to analyze MS raw files. MS/MS spectra were searched against the E. coli Nissle Uniprot FASTA database (UP000011176, the sequence for argA^{fbr} was manually added) and a common contaminants database (247 entries) by the Andromeda search engine. A false discovery rate (FDR) of 1% was required for peptides and proteins. Peptide identification was performed with an allowed initial precursor mass deviation of up to 7 ppm and an allowed fragment mass deviation of 20 ppm. Nonlinear retention time alignment of all measured samples was performed in MaxQuant. Peptide identifications were matched across different replicates within a time window of 1 min of the aligned retention times. Protein identification required at least 1 razor peptide. A minimum ratio count of 1 was required for valid quantification events via MaxQuant's Label Free Quantitation algorithm (MaxLFQ). Data were filtered for common contaminants and peptides only identified by side modification were excluded from further analysis.

Data analysis was performed using the R statistical computing environment. Missing values were imputed with a normal distribution of 30% in comparison to the SD of measured values and a 1.8 SD down-shift of the mean to simulate the distribution of low signal values.

1. A method of treating a tumor in a subject, the method comprising administering to the subject an arginine-producing bacterium in combination with a checkpoint inhibitor, wherein the combination of the bacterium and the checkpoint inhibitor causes a synergistic therapeutic effect on the tumor in the subject, and wherein the bacterium is capable of producing at least about 300 μM arginine in culture in vitro after about 3 hours,
thereby treating the tumor in the subject.

2. The method of claim 1, wherein the bacterium is also an ammonia-consuming bacterium.

3. A method of treating a tumor in a subject, the method comprising administering to the subject an ammonia-consuming bacterium in combination with a checkpoint inhibitor, wherein the combination of the bacterium and the checkpoint inhibitor causes a synergistic therapeutic effect on the tumor in the subject, and wherein the bacterium is capable of producing at least about 300 μM arginine in culture in vitro after about 3 hours,
thereby treating the tumor in the subject.

4. The method of claim 3, wherein the bacterium is also an arginine-producing bacterium.

5. (canceled)

6. The method of claim 1, wherein the bacterium is capable of producing at least about 100 μM arginine in culture in vitro after about 1.5 hours.

7. The method of claim 1, wherein the bacterium produces between about 10 μM to about 600 μM arginine in culture in vitro between about 0.5 hours to about 3 hours.

8. The method of claim 1, wherein the bacterium produces at least about 3 μg of arginine per gram of tumor.

9. The method of claim 1, wherein the bacterium produces between at least about 3 μg of arginine per gram of tumor to at least about 25 μg of arginine per gram of tumor.

10. The method of claim 1, wherein the bacterium comprises a deletion in an endogenous arginine repressor gene and expresses at least one exogenous arginine biosynthetic enzyme under the control of an inducible promoter.

11. The method of claim 1, wherein the bacterium comprises a deletion of an argR gene and insertion of an argA gene.

12. The method according to claim 11, wherein the argA gene is argA^{fbr}.

13. The method of claim 1, wherein the synergistic therapeutic effect is:
i) a decrease in tumor volume,
ii) a decrease in tumor weight,
iii) inhibition of tumor growth,
iv) partial eradication of the tumor, and/or
v) complete eradication of the tumor.

14. The method of claim 13, wherein the synergistic therapeutic effect is a decrease in tumor volume of at least about 100 mm³ after about 30 days of administration.

15. The method of claim 13, wherein the synergistic therapeutic effect is a decrease in tumor weight of at least two-fold after about 30 days of administration.

16. The method of claim 13, wherein the synergistic therapeutic effect is inhibition of tumor growth for at least 30 days after administration.

17. The method of claim 13, wherein the synergistic therapeutic effect is partial eradication of the tumor.

18. The method of claim 13, wherein the synergistic therapeutic effect is a complete eradication of the tumor.

19. The method of claim 1, wherein the combination of the bacterium and the checkpoint inhibitor causes the synergistic therapeutic effect on the tumor in the subject as compared to a therapeutic effect caused by administering the bacterium alone, or administering the checkpoint inhibitor alone, to a subject.

20. The method of claim 13, wherein the subject is a population of subjects, and wherein at least 35% of the subjects in the population of subjects exhibit partial eradication of the tumor.

21. The method of claim 13, wherein the subject is a population of subjects, and wherein at least 35% of the subjects in the population of subjects exhibit complete eradication of the tumor.

22. The method of claim 1, wherein administering the bacterium and the checkpoint inhibitor is concurrent or sequential.

23. The method of claim 1, further comprising selecting a subject who would benefit from an increase in therapeutic efficacy of the checkpoint inhibitor.

24. A method of increasing T-cell infiltration into a tumor in a subject, the method comprising
administering to the subject an arginine-producing bacterium in combination with a checkpoint inhibitor, wherein the combination of the arginine-producing bacterium and the checkpoint inhibitor increases T cell infiltration into the tumor in the subject at least two-fold as compared to the T cell infiltration exhibited by administering the bacterium alone, or the checkpoint inhibitor alone, and wherein the bacterium is capable of producing at least about 300 μM arginine in culture in vitro after about 3 hours, to a subject.

25. The method of claim 24, wherein the combination of the bacterium and the checkpoint inhibitor causes a synergistic therapeutic effect on the tumor in the subject as compared to a therapeutic effect caused by administering the bacterium alone, or the checkpoint inhibitor alone, to a subject.

26. The method of claim 24, wherein arginine levels in the tumor microenvironment (TME) are increased and ammonia levels in the TME are decreased.

27. The method of claim 26, wherein the arginine levels in the tumor microenvironment (TME) are increased to greater than 30 μg of arginine per gram of tumor.

28. The method of claim 24, wherein a T-cell response against the tumor is enhanced.

29. The method of claim 28, wherein the tumor is colonized by at least about 8,000 CD4⁺ T-cells per gram of tumor tissue.

30. The method of claim 28, wherein the tumor is colonized by at least about 11,000 CD8⁺ T-cells per gram of tumor tissue.

31. The method of claim 1, wherein the bacterium is an engineered bacterium.


SEQUENCE LISTING (41 sequences)

SEQ ID NO: 1 - DNA, 60 bp, Escherichia coli
gcaaaaaaacagaataaaaatacaataatttcgaataatcatgcaaagaggtgtaccgtg

SEQ ID NO: 2 - DNA, 60 bp, Escherichia coli
gcaaaaaaacactttaaaaacttaataatttcctttaatcacttaaagaggtgtaccgtg

SEQ ID NO: 3 - DNA, 76 bp, Escherichia coli
agacttgcaaatgaataatcatccatatagattgaattttaattcattaaggcgttagccacaggagggatctatg

SEQ ID NO: 4 - DNA, 76 bp, Escherichia coli
agacttgcaaacttatacttatccatatagattttgttttaatttgttaaggcgttagccacaggagggatctatg

SEQ ID NO: 5 - DNA, 160 bp, Escherichia coli
tcattgttgacacacctctggtcatgatagtatcaatattcatgcagtatttatgaataaaaatacactaacgttgagcgtaataaaacccaccagccgtaaggtgaatgttttacgtttaacctggcaaccagacataagaaggtgaatagccccgatg

SEQ ID NO: 6 - DNA, 160 bp, Escherichia coli
tcattgttgacacacctctggtcatgatagtatcaaacttcatgggatatttatctttaaaaatacttgaacgttgagcgtaataaaacccaccagccgtaaggtgaatgttttacgtttaacctggcaaccagacataagaaggtgaatagccccgatg

SEQ ID NO: 7 - DNA, 160 bp, Escherichia coli
catcggggctattcaccttcttatgtctggttgccaggttaaacgtaaaacattcaccttacggctggtgggttttattacgctcaacgttagtgtatttttattcataaatactgcatgaatattgatactatcatgaccagaggtgtgtcaacaatga

SEQ ID NO: 8 - DNA, 160 bp, Escherichia coli
catcggggctattcaccttcttatgtctggttgccaggttaaacgtaaaacattcaccttacggctggtgggttttattacgctcaacgttcaagtatttttaaagataaatatcccatgaagtttgatactatcatgaccagaggtgtgtcaacaatga

SEQ ID NO: 9 - DNA, 81 bp, Escherichia coli
agcagatttgcattgatttacgtcatcattgtgaattaatatgcaaataaagtgagtgaatattctctggagggtgttttg

SEQ ID NO: 10 - DNA, 81 bp, Escherichia coli
agcagatttgcattgatttacgtcatcattgtcttttaatatcttaataactggagtgacgtttctctggagggtgttttg

SEQ ID NO: 11 - DNA, 88 bp, Escherichia coli
tttctgattgccattcagtgattttttatgcatattttgtgattataatttcatatttatttatgcgtaacagggtgatcatgagatg

SEQ ID NO: 12 - DNA, 88 bp, Escherichia coli
tttctgattgccattcagtctttttttacttatattttgtctttataatcttatatttatttatgcgtaacagggtgatcatgagatg

SEQ ID NO: 13 - DNA, 219 bp, Escherichia coli
ctaatcacgtgaatgaatatccagttcactttcatttgttgaatacttttaccttctcctgctttcccttaagcgcattattttacaaaaaacacactaaactcttcctgtctccgataaaagatgattaaatgaaaactcatttattttgcataaaaattcagtgaaagcagaaatccaggctcatcatcagttaattaagcagggtgttattttatg

SEQ ID NO: 14 - DNA, 219 bp, Escherichia coli
ctaatcaccttaatgaatcttcagttcactttcatttgttgaatacttttaccttctcctgctttcccttaagcgcattattttacaaaaaacacactaaactcttcctgtctccgataaaagatgatcttatgaaaacctttttatttcttataaaaatcttgtgaaagcagaaatccaggctcatcatcagttaattaagcagggtgttattttatg

SEQ ID NO: 15 - DNA, 233 bp, Escherichia coli
cctgaaacgtggcaaattctactcgttttgggtaaaaaatgcaaatactgctgggatttggtgtaccgagacgggacgtaaaatctgcaggcattatagtgatccacgccacattttgtcaacgtttattgctaatcattgacggctagctcagtcctaggtacagtgctagcacccgtttttttgggctagaaataattttgtttaactttaagaaggagatatacataccc

SEQ ID NO: 16 - DNA, 1053 bp, Escherichia coli
gtgatccacgccacattttgtcaacgtttattgctaatcacgtgaatgaatatccagttcactttcatttgttgaatacttttaccttctcctgctttcccttaagcgcattattttacaaaaaacacactaaactcttcctgtctccgataaaagatgattaaatgaaaactcatttattttgcataaaaattcagtgaaagcagaaatccaggctcatcatcagttaattaagcagggtgttattttatgacgacgattctcaagcatctcccggtaggtcaacgtattggtatcgctttttccggcggtctggacaccagtgccgcactgctgtggatgcgacaaaagggagcggttccttatgcatatactgcaaacctgggccagccagacgaagaggattatgatgcgatccctcgtcgtgccatggaatacggcgcggagaacgcacgtctgatcgactgccgcaaacaactggtggccgaaggtattgccgctattcagtgtggcgcatttcataacaccactggtggactgacctatttcaacacgacgccgctgggccgcgccgtgaccggcaccatgctggttgctgctatgaaagaagatggcgtgaatatctggggtgacggcagcacctataaaggaaacgatatcgaacgtttctaccgttacggtctgctgaccaatgctgaactgcagatttacaaaccgtggcttgatactgactttattgatgaactgggtggccgtcatgagatgtctgaatttatgattgcctgcggtttcgactacaaaatgtctgtcgaaaaagcttactccacggactccaacatgcttggtgcaacgcatgaagcgaaggatctggaatacctcaactccagcgtcaaaatcgtcaacccaattatgggcgtgaagttttgggatgagagcgtgaaaatcccggcagaagaagtcacagtacgctttgagcaaggtcatccggtggcgctgaacggtaaaacctttagcgacgacgtagaaatgatgctggaagctaaccgcatcggc

SEQ ID NO: 17 - DNA, 734 bp, Escherichia coli
ttgacggctagctcagtcctaggtacagtgctagcacccgtttttttgggctagaaataattttgtttaactttaagaaggagatatacatacccatgacgacgattctcaagcatctcccggtaggtcaacgtattggtatcgctttttccggcggtctggacaccagtgccgcactgctgtggatgcgacaaaagggagcggttccttatgcatatactgcaaacctgggccagccagacgaagaggattatgatgcgatccctcgtcgtgccatggaatacggcgcggagaacgcacgtctgatcgactgccgcaaacaactggtggccgaaggtattgccgctattcagtgtggcgcatttcataacaccactggtggactgacctatttcaacacgacgccgctgggccgcgccgtgaccggcaccatgctggttgctgctatgaaagaagatggcgtgaatatctggggtgacggcagcacctataaaggaaacgatatcgaacgtttctaccgttacggtctgctgaccaatgctgaactgcagatttacaaaccgtggcttgatactgactttattgatgaactgggtggccgtcatgagatgtctgaatttatgattgcctgcggtttcgactacaaaatgtctgtcgaaaaagcttactccacggactccaacatgcttggtgcaacgcatgaagcgaaggatctggaatacctcaactccagc

SEQ ID NO: 18 - DNA, 117 bp, Artificial Sequence
  Description of Artificial Sequence Synthetic polynucleotide
atccccatcactcttgatggagatcaattccccaagctgctagagcgttaccttgcccttaaacattagcaatgtcgatttatcagagggccgacaggctcccacaggagaaaaccg

SEQ ID NO: 19 - DNA, 108 bp, Artificial Sequence
  Description of Artificial Sequence Synthetic polynucleotide
ctcttgatcgttatcaattcccacgctgtttcagagcgttaccttgcccttaaacattagcaatgtcgatttatcagagggccgacaggctcccacaggagaaaaccg

SEQ ID NO: 20 - DNA, 290 bp, Artificial Sequence
  Description of Artificial Sequence Synthetic polynucleotide
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 21 - DNA, 433 bp, Artificial Sequence
  Description of Artificial Sequence Synthetic polynucleotide
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 22 - DNA, 290 bp, Artificial Sequence
  Description of Artificial Sequence Synthetic polynucleotide
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 23 - DNA, 173 bp, Artificial Sequence
  Description of Artificial Sequence Synthetic polynucleotide
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 24 - DNA, 305 bp, Artificial Sequence
  Description of Artificial Sequence Synthetic polynucleotide
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 25 - DNA, 180 bp, Artificial Sequence
  Description of Artificial Sequence Synthetic polynucleotide
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 26 - DNA, 199 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 27 - DNA, 207 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 28 - DNA, 390 bp, Artificial Sequence
  Description of Artificial Sequence Synthetic polynucleotide
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 29 - DNA, 200 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 30 - DNA, 1332 bp, Artificial Sequence
  Description of Artificial Sequence Synthetic polynucleotide
atggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaa

SEQ ID NO: 31 - protein, 443 aa, Artificial Sequence
  Description of Artificial Sequence Synthetic polypeptide
MVKERKTELVEGFRHSVPCINTHRGKTFVIMLGGEAIEHENFSSIVNDIGLLHSLGIRLVVVYGARPQIDANLAAHHHEPLYHKNIRVTDAKTLELVKQAAGTLQLDITARLSMSLNNTPLQGAHINVVSGNFIIAQPLGVDDGVDYCHSGRIRRIDEDAIHRQLDSGAIVLMGPVAVSVTGESFNLTSEEIATQLAIKLKAEKMIGFCSSQGVTNDDGDIVSELFPNEAQARVEAQEEKGDYNSGTVRFLRGAVKACRSGVRRCHLISYQEDGALLQELFSRDGIGTQIVMESAEQIRRATINDIGGILELIRPLEQQGILVRRSREQLEMEIDKFTIIQRDNTTIACAALYPFPEEKIGEMACVAVHPDYRSSSRGEVLLERIAAQAKQSGLSKLFVLTTRSIHWFQERGFTPVDIDLLPESKKQLYNYQRKSKVLMADLG

SEQ ID NO: 32 - DNA, 1539 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaa

SEQ ID NO: 33 - DNA, 5417 bp, Artificial Sequence
  Description of Artificial Sequence Synthetic polynucleotide
gtaaaacgacggccagtgaattcgagctcggtaccatccccatcactcttgatggagatcaattccccaagctgctagagcgttaccttgcccttaaacattagcaatgtcgatttatcagagggccgacaggctcccacaggagaaaaccgatggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaaacagaataaaaatacaataatttcgaataatcatgcaaagcttggcgtaatcatggtcatagctgtttcctgtgtgaaattgttatccgctcacaattccacacaacatacgagccggaagcatgtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgtt

SEQ ID NO: 34 - DNA, 3995 bp, Artificial Sequence
  Description of Artificial Sequence Synthetic polynucleotide
attaagttgggtaacgccagggttttcccagtcacgacgttattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcg

SEQ ID NO: 35 - DNA, 5660 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaaggaagtttgtctagatctcaggcgtggatggcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcg

SEQ ID NO: 36 - DNA, 3115 bp, Artificial Sequence
  Description of Artificial Sequence Synthetic polynucleotide
ctacgccccatcgttgctttgtgtgatctctgttacagaattggcggtaatgtggagatgcgcacataaaatcgccatgatttttgcaagcaacatcacgaaattccttacatgacctcggtttagttcacaggacgtcccatggctcgagcatgcgagagtagggaactgccaggcatcaaataaaatgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggaccatggctaattcccaggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaagtcgacgcatgcatcgataagccgcgttctcatcctcccgcctcctcccccataaaaaagccagggggtggaggatttaagccatctcctgatgac

SEQ ID NO: 37 - DNA, 3583 bp, Artificial Sequence
  Description of Artificial Sequence Synthetic polynucleotide
ctacgccccatcgttgctttgtgtgatctctgttacagaattggcggtaatgtggagatgcgcacataaaatcgccatgatttttgcaagcaacatcacgaaattccttacatgacctcggtttagttcacaggacgtcccatggctcgagcatgcgagagtagggaactgccaggcatcaaataaaatgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggctaattcccagatatggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaagtcgacgcatgcatcgataagccgcgttctcatcctcccgcctcctcccccataaaaaagccagggggtggaggatttaagccatctcctgatgac

SEQ ID NO: 38 - DNA, 2185 bp, Artificial Sequence
  Description of Artificial Sequence Synthetic polynucleotide
ctacgccccatcgttgctttgtgtgatctctgttacagaattggcggtaatgtggagatgcgcacataaaatcgccatgatttttgcaagcaacatcacgaaattccttacatgacctcggtttagttcacaggacgtcccatggctcgagcatgcgagagtagggaactgccaggcatcaaataaaatgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaactaaggaggatattcatatggaccatggctaattcccaggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaagtcgacgcatgcatcgataagccgcgttctcatcctcccgcctcctcccccataaaaaagccagggggtggaggatttaagccatctcctgatgac

SEQ ID NO: 39 - DNA, 18 bp, Artificial Sequence
  Description of Artificial Sequence Synthetic consensus sequence
  modified_base (2)..(2): a, c, t, g, unknown or other
  modified_base (17)..(17): a, c, t, g, unknown or other
wntgaatwwwwattcanw

SEQ ID NO: 40 - DNA, 14 bp, Artificial Sequence
  Description of Artificial Sequence Synthetic consensus sequence
  modified_base (6)..(9): a, c, t, g, unknown or other
ttgatnnnnatcaa

SEQ ID NO: 41 - DNA, 471 bp, Artificial Sequence
  Description of Artificial Sequence Synthetic polynucleotide
atgcgaagctcggctaagcaagaagaactagttaaagcatttaaagcattacttaaagaagagaaatttagctcccagggcgaaatcgtcgccgcgttgcaggagcaaggctttgacaatattaatcagtctaaagtctcgcggatgttgaccaagtttggtgctgtacgtacacgcaatgccaaaatggaaatggtttactgcctgccagctgaactgggtgtaccaaccacctccagtccattgaagaatctggtgctggatatcgactacaacgatgcagttgtcgtgattcataccagccctggcgcggcgcagttaattgctcgcctgctggactcactgggcaaagcagaaggtattctgggcaccatcgctggcgatgacaccatctttaccacccctgctaacggtttcacagtcaaagacctgtacgaagcgattttagagctgttcgaccaggagctttaa